(19) United States
(12) Patent Application Publication
(10) Pub. No.: US 2020/0261549 A1
(43) Pub. Date: Aug. 20, 2020

(54) METHODS OF TREATING AND PROTECTING AGAINST CARDIAC DISEASE, CARDIOVASCULAR DISEASE AND RELATED CONDITIONS AND SYMPTOMS

(71) Applicant: AlphaCore Pharma LLC, Ann Arbor, MI (US)

(72) Inventors: Richard Thomas GEORGE, Jr., Gaithersburg, MD (US); Chao-Yu JIN, Gaithersburg, MD (US)

(21) Appl. No.: 16/762,063

(22) PCT Filed: Nov. 5, 2018
(86) PCT No.: PCT/IB18/58683

§ 371 Date: May 6, 2020

Related U.S. Application Data
(60) Provisional application No. 62/629,900, filed on Feb. 13, 2018.
(60) Provisional application No. 62/582,382, filed on Nov. 7, 2017.

(51) Int. Cl. A61K 38/45; A61K 9/00; A61P 9/10

(52) U.S. Cl. CPC A61K 38/45; A61P 9/10; A61K 9/0019; C12Y 203/01043

(57) ABSTRACT

Provided are methods of treating subjects having, or at risk of having, heart disease, cardiovascular disease, coronary artery disease, cerebrovascular disease, acute or chronic renal disease, and/or symptoms thereof with certain therapeutically effective doses and dosing regimens comprising an isolated and purified lecithin-cholesterol acyltransferase (LCAT) enzyme, in particular, a recombinant human LCAT (rhLCAT) enzyme, e.g., MEDI6012. The methods involving administration of the described doses of rhLCAT increase serum levels of high density lipoprotein (HDL) and apolipoprotein A1 (apoA1) and decrease, or do not appreciably increase, serum levels of apolipoprotein B in the treated subjects, thus affording treatment of heart disease, heart-related diseases and coronary artery disease. The methods involving the described dosing regimens and doses of rhLCAT or MEDI6012 administered to a subject further provide cardio-, myocardio- and cardiovascular protection for the subject, including protection against ischemic stroke, myocardiocyte apoptosis, atherosclerosis progression and the like.

### BACKGROUND OF THE INVENTION

According to The World Health Organization, cardiovascular diseases are the leading causes of morbidity and mortality worldwide. Approximately 17.7 million fatalities from cardiovascular diseases occurred worldwide in 2015, representing 31 percent of all global deaths for that year. Of these deaths, an estimated 7.4 million were caused by coronary heart disease (CHD) and 6.7 million were caused by stroke. Heart disease remains the leading cause of mortality for both men and women of most ethnicities in the United States, with about 630,000 deaths occurring every year, according to the Centers for Disease Control and Prevention. Coronary heart disease is the most common type of heart disease in the U.S; it accounted for over 360,000 deaths in 2015. Heart disease and associated coronary diseases and syndromes are projected to remain the leading causes of global mortality over the next decade and beyond.

Heart and coronary artery diseases affect not only cardiovascular disease patients, but also pose a serious health problem for rising numbers of individuals who suffer from metabolic disorders, such as obesity and/or diabetes, which frequently lead to increased cardiovascular risk. Heart disease and related health conditions take an enormous economic toll, as costs for health care services, medications and lost productivity of afflicted individuals total about $200 billion dollars in the U.S. each year.

Atherosclerosis in humans is a pathological condition that is characterized by the accumulation of cholesterol in the arteries. Cholesterol accumulates in the foam cells residing in the wall of arteries, thereby narrowing the lumen of these vessels and causing decreased blood flow. The development of atherosclerosis is inversely related to the concentration of high density lipoproteins (HDL) in the serum, for example, low concentrations of HDL are associated with an increased risk of cardiovascular disease.

Lecithin-cholesterol acyltransferase (LCAT), a plasma enzyme secreted by the liver, catalyzes the production of cholesteryl ester (CE) from free cholesterol and phosphatidylcholine (lecithin). LCAT has been proposed to play a role in the process of reverse cholesterol transport (RCT). In the first step of RCT, free cholesterol effluxes from macrophages in plaque by the adenosine triphosphate (ATP)-binding cassette A1 (ABCA1) to plasma acceptors, such as preβ-HDL, and other smaller particle forms of HDL. LCAT converts free cholesterol on HDL to CE, increases the capacity of HDL to remove additional cholesterol from tissues, and maintains the gradient for cholesterol efflux from cells. While the role of LCAT in the RCT process is consistent with a finding of low LCAT activity and increased preβ-HDL in patients with heart disease, contradictory data and findings exist regarding the functional inter-relationships among HDL, LCAT and heart disease in patients.

Given the ever-increasing numbers of individuals afflicted with heart and coronary artery diseases and their impact on a global scale, treatment methods that reduce or alleviate heart diseases of different types, including coronary artery disease, and the risk for these conditions, are urgently needed. The newly developed therapeutic and protective treatment methods described herein provide vital and essential therapies for individuals with acute and chronic heart disease, CHD, coronary artery disease, and the like.

### SUMMARY OF THE INVENTION

As described below, the present disclosure features therapeutic and preventive methods of treating a subject, particularly a mammalian subject, and more particularly, a human subject, who has chronic or acute heart disease, heart-related diseases, coronary heart disease, cardiovascular disease, cerebrovascular disease, atherosclerotic disease and/or symptoms thereof with effective doses and dosing regimens of an isolated and purified lecithin-cholesterol acyltransferase (LCAT) enzyme, in particular, isolated and purified human LCAT, or recombinantly produced (recombinant) human LCAT (rhLCAT), called MEDI6012 herein. The described methods embrace the use of an LCAT enzyme, in particular, a human LCAT enzyme, that is isolated and purified from its naturally occurring environment or recombinant cellular materials. An isolated and purified human LCAT enzyme encompasses a rhLCAT enzyme. In a particular embodiment, the rhLCAT enzyme is called MEDI6012 herein. It will be appreciated that the terms “isolated and purified human LCAT,” “LCAT,” “rhLCAT” and MEDI6012 may be used interchangeably herein.

In the body, the LCAT enzyme is found in the bloodstream and exists as a key factor in the reverse cholesterol transport (RCT) system, which is believed to have significant importance in the elimination of excess cholesterol from the body. LCAT is also considered to be pivotal in systemically managing high-density lipoprotein (HDL), or “good” cholesterol, levels in serum and plasma. Because the process of accumulating cholesterol in both cardiovascular and peripheral arteries can lead to atherosclerosis and its clinical sequelae (such as, for example, heart attack (also known as myocardial infarction (MI)), ischemic heart disease, stroke, ischemic stroke, peripheral vascular disease and the symptoms thereof), reducing, slowing, or reversing the process of systemic cholesterol accumulation in the body is effective in the treatment or prevention of heart disease and atherosclerosis.

The methods described herein afford therapeutic treatment benefit for, as well as protective effects against, heart disease, heart-related conditions and diseases, and cardiovascular disease and their symptoms by providing doses and dosing regimens of an isolated and purified LCAT enzyme that is administered to subjects so as to increase systemically the level of LCAT activity in the sera (or plasma) of treated subjects and, in turn, reduce (e.g., slow, decrease, or reverse) the accumulation of free or unesterified cholesterol in the arteries of the subjects undergoing treatment. Moreover, the present methods offer other cardiotherapeutic, cardioprotective, and anti-atherogenic (atheroprotective) effects and myocardioprotective effects by preventing myocardial fibrosis and hypertrophy in a subject as described herein.

In accordance with the present disclosure, the use of the isolated and purified LCAT enzyme, i.e., rhLCAT or MEDI6012, in the effective dosage amounts and dosing regimens described herein and practiced in the present methods provides both first-line treatment for a subject with the aforementioned cardiac and cardiovascular diseases and/or the symptoms thereof, and effective maintenance therapy and treatment for subjects with various forms of these diseases and symptoms. The methods described herein offer advantages to standard-of-care (SoC) treatment for heart disease, cardiac-related and cardiovascular diseases and conditions and may also provide therapeutic treatment benefits for subjects who have relapsed following another cardiac or cardiovascular therapy regimen.

Provided in one aspect described herein is a method of treating heart disease or cardiovascular disease and/or the symptoms thereof in a subject, in which the method comprises administering to a subject in need thereof one or more doses of an isolated and purified LCAT enzyme in an amount of 20-2000 mg over a time period of about or equal to 1 minute to 3 hours, to treat heart disease or cardiovascular disease and/or the symptoms thereof in the subject. In an embodiment of the method, the isolated and purified LCAT enzyme is a recombinant human LCAT (rhLCAT) enzyme, or MEDI6012. In embodiments of the method, the subject has acute or chronic heart disease, cardiovascular disease, coronary artery disease (CAD), stable CAD, atherosclerosis, atherosclerotic cardiovascular disease (CVD), stable CVD, unstable CVD, acute coronary syndrome (ACS), heart failure (HF), congestive HF, hospitalized HF, heart failure with reduced ejection fraction (EF), heart failure with preserved EF, ST-elevated myocardial infarction (STEMI), non-STEMI, or a disease, pathology, or condition related to or associated with heart or cardiac disease, familial or acquired, such as stroke, ischemic stroke, myocardial disease, peripheral artery disease, myocardial infarction, ischemic cardiomyopathy, non-ischemic cardiomyopathy, chemotherapy-induced cardiomyopathy, cerebrovascular disease, acute or chronic renal disease, and/or symptoms thereof. In a particular embodiment, the subject has stable coronary artery disease (CAD). In particular embodiments of the method, the one or more doses of the LCAT enzyme administered to the subject are in an amount selected from 20 mg, 24 mg, 40 mg, 80 mg, 100 mg, 150 mg, 240 mg, 300 mg, 600 mg, 800 mg or 1600 mg. In other particular embodiments of the method, the one or more doses of the LCAT enzyme are administered to the subject in an amount selected from 300 mg, 150 mg and 100 mg. In an embodiment of the method, the one or more doses of LCAT comprise a first dose in an amount of 300 mg and a second dose in an amount of 150 mg administered about 48 hours±8 hours following the first dose. In another embodiment of the method, the one or more doses of LCAT comprise a first dose in an amount of 300 mg; a second dose in an amount of 150 mg administered about 48 hours±8 hours following the first dose; and a third dose in an amount of 100 mg administered about a week following the second dose. In yet another embodiment of the method, the one or more doses of LCAT comprise a first dose in an amount of 300 mg; a second dose in an amount of 150 mg administered about 48 hours±8 hours following the first dose; and at least four subsequent doses in amounts of 100 mg per dose administered approximately weekly following the second dose. In an embodiment of the method, the one or more doses of LCAT are administered intravenously to the subject. In an embodiment, the one or more doses of LCAT are administered to the subject via an IV push. In an embodiment, LCAT is administered intravenously to the subject over a time period of about 30 minutes to 1 hour. In an embodiment, LCAT is administered by IV push to the subject over a time period of about 1-3 minutes. In another embodiment, LCAT is administered to the subject in one dose in an amount of 300 mg. In an embodiment, the one dose of LCAT is administered to the subject by IV push within a time period of about 1-3 minutes. In another embodiment, LCAT is administered to the subject in two doses in which the first dose is in an amount of 300 mg and the second dose is in an amount of 150 mg. In another embodiment, LCAT is administered to the subject in three doses in which the first dose is in an amount of 300 mg; the second dose is in an amount of 150 mg; and the third dose is in an amount of 100 mg. In another embodiment, LCAT is administered to the subject in six doses in which the first dose is in an amount of 300 mg; the second dose is in an amount of 150 mg; and the third to sixth doses are in an amount of 100 mg. In an embodiment of the method, the second dose of LCAT is administered to the subject within about 48 hours±8 hours after the first dose. In another embodiment of the method, the third dose of LCAT is administered to the subject within about a week after the second dose. In an embodiment, the second dose of LCAT is administered to the subject within about 48 hours±8 hours after the first dose; the third dose of LCAT is administered to the subject within about a week after the second dose; and the fourth through sixth doses of LCAT are administered approximately weekly thereafter. In an embodiment of the foregoing, at least the first dose of LCAT is administered to the subject by IV push. In another embodiment, LCAT is administered to the subject via subcutaneous (SC) injection, e.g., at a dose of 80 mg or 600 mg. In an embodiment, the administration of LCAT by SC injection at a dose of 600 mg increases endogenous levels of apolipoprotein A1 (apoA1) in the subject. In another embodiment of the above described method, the administration of LCAT increases endogenous levels of high density lipoprotein-cholesterol (HDL-C) and/or apolipoprotein A1 (apoA1) in the subject. In another embodiment of the method, the administration of LCAT does not increase endogenous levels of apolipoprotein B (apoB) in the subject. In an embodiment of any of the foregoing, the isolated and purified LCAT is recombinant human LCAT (rhLCAT). In a particular embodiment, the rhLCAT is MEDI6012 (SEQ ID NO: 2).

Provided in another aspect described herein is a method of treating heart disease or cardiovascular disease and/or the symptoms thereof in a subject, in which the method comprises administering to a subject in need thereof a loading dose of an isolated and purified LCAT enzyme in an amount of 250-500 mg delivered to the subject by intravenous (IV) push over a time period of about 1-5 minutes upon presentation of the subject for treatment. In an embodiment of the method, the loading dose of LCAT is administered to the subject in an amount of 300 mg. In another embodiment of the method, the loading dose of LCAT is administered to the subject over a time period of about 1-3 minutes. In another embodiment of the method, the loading dose of LCAT is administered to the subject over a time period of about 1 minute. In another embodiment of the method, one or more doses of LCAT are administered to the subject following the loading dose. In an embodiment, a dose of LCAT in an amount of 100-200 mg is administered to the subject following the loading dose. In another embodiment, a dose of LCAT in an amount of 150 mg is administered to the subject following the loading dose. In another embodiment, a dose of LCAT in an amount of 100-150 mg is administered to the subject following the 100-200 mg or the 150 mg dose. In another embodiment, a dose of LCAT in an amount of 100 mg is administered to the subject following the 100-200 mg or the 150 mg dose. In another embodiment of the method, at least 4 weekly doses of LCAT in an amount of 80-150 mg per dose are administered to the subject about a week following the 100-200 mg dose or the 150 mg dose. In another embodiment of the method, at least 4 weekly doses of LCAT in an amount of 100 mg per dose are administered to the subject about a week following the 100-200 mg dose or the 150 mg dose. In another embodiment, the one or more doses of LCAT following the loading dose are intravenously administered to the subject. In an embodiment of the method, the isolated and purified LCAT is recombinant human LCAT (rhLCAT). In a particular embodiment, the rhLCAT is MEDI6012 (SEQ ID NO: 2).

As will be appreciated by the skilled practitioner, the described methods which include a loading dose of the LCAT enzyme, such as rhLCAT or MEDI6012, and particularly, a loading dose administered to a subject as an IV push over 1-3 minutes, facilitates the treatment of diseases and conditions where time is of the essence. As described and exemplified herein, administering the described doses and dose regimens of rhLCAT or MEDI6012, including a loading dose, to a patient can increase HDL-C levels in the patient within minutes. As such, the present methods provide doses of active agent, rhLCAT or MEDI6012, that rapidly increase levels of endogenous products such as HDL-C and/or apoA1 to achieve therapeutic and protective treatment of a patient who presents with acute disease, such as, without limitation, acute MI, stroke, or kidney injury. Accordingly, rhLCAT or MEDI6012 administration is highly advantageous and optimal for patients who need immediate treatment of acute disease, pathology, or injury on an urgent care basis.

Provided in another aspect described herein is a method of treating heart disease or cardiovascular disease and/or the symptoms thereof in a subject, in which the method comprises parenterally administering to a subject in need thereof two or more doses of an isolated and purified LCAT enzyme, wherein each dose comprises LCAT in an amount of 20-500 mg to treat heart disease or cardiovascular disease and/or the symptoms thereof in the subject. In an embodiment of the method, the two or more doses of LCAT administered to the subject are in an amount selected from 300 mg, 150 mg, or 100 mg. In an embodiment, three doses of LCAT are administered to the subject and comprise a dose of 300 mg administered on day 1; a dose of 150 mg administered on day 3; a dose of 100 mg administered on day 10; and optionally wherein subsequent doses of LCAT are administered to the subject at predetermined time intervals up to about 30 days, or longer, following the day 10 dose. In certain other embodiments, the subsequent doses of LCAT, e.g., 6 doses as described herein, are administered to the subject at predetermined time periods, e.g., weekly, following the day 10 dose. In an embodiment, LCAT is intravenously administered to the subject by intravenous push and/or intravenous infusion. In an embodiment, the subject has acute or chronic heart disease, cardiovascular disease, coronary artery disease (CAD), stable CAD, atherosclerosis, atherosclerotic cardiovascular disease (CVD), stable CVD, unstable CVD, acute coronary syndrome (ACS), stroke, ischemic stroke, myocardial disease, myocardial infarction, familial or acquired, heart failure with reduced ejection fraction (EF), heart failure with preserved EF, non-ischemic cardiomyopathy, chemotherapy-induced cardiomyopathy, cerebrovascular disease, acute or chronic renal disease and/or symptoms thereof. In an embodiment, the isolated and purified LCAT is recombinant human LCAT (rhLCAT). In a particular embodiment, the rhLCAT is MEDI6012 (SEQ ID NO: 2). In another particular embodiment, the treated subject has stable CVD.

Provided in yet another aspect described herein is a method of treating heart disease or cardiovascular disease and/or the symptoms thereof in a subject, in which the method comprises administering intravenously to a subject in need thereof a first dose of an isolated and purified lecithin-cholesterol acyltransferase (LCAT) enzyme in an amount of 200-500 mg; and administering intravenously to the subject a second dose of the LCAT enzyme in an amount of 100-200 at approximately 48 hours±8 hours following the first dose, to treat heart disease or cardiovascular disease and/or the symptoms thereof in the subject. In an embodiment of the method, the first dose of LCAT is 300 mg and the second dose of LCAT is 100 mg or 150 mg. In a particular embodiment, the first dose of LCAT is 300 mg and the second dose of LCAT is 150 mg. In an embodiment of the method, at least the first dose of LCAT is administered to the subject by IV push. In a particular embodiment of the method, the administration by IV push is over a time period of about 1-3 minutes. In an embodiment, the method further comprises administering intravenously to the subject a dose of LCAT in an amount of 100-150 mg about a week following the second dose. In a particular embodiment, the dose of LCAT administered to the subject is an amount of 100 mg about a week following the second dose. In an embodiment, the method further comprises administering intravenously to the subject at least four weekly doses of LCAT in an amount of 100-200 mg following the second dose. In a particular embodiment, the at least four weekly doses of LCAT are in an amount of 100 mg following the second dose. In an embodiment, the isolated and purified LCAT is recombinant human LCAT (rhLCAT). In a particular embodiment, the rhLCAT is MEDI6012 (SEQ ID NO: 2). In embodiments of the method, the subject has acute or chronic heart disease, cardiovascular disease, coronary artery disease (CAD), stable CAD, atherosclerosis, atherosclerotic cardiovascular disease (CVD), stable CVD, unstable CVD, acute coronary syndrome (ACS), stroke, ischemic stroke, myocardial disease, myocardial infarction, familial or acquired, heart failure with reduced ejection fraction (EF), heart failure with preserved EF, non-ischemic cardiomyopathy, chemotherapy-induced cardiomyopathy, cerebrovascular disease, acute or chronic renal disease and/or symptoms thereof.

Provided in yet another aspect described herein is a method of treating heart disease or cardiovascular disease and/or the symptoms thereof in a subject, in which the method comprises administering to a subject in need thereof a first dose of isolated and purified lecithin-cholesterol acyltransferase (LCAT) enzyme MEDI6012 in an amount of 200-500 mg; administering intravenously to the subject a second dose of the LCAT enzyme MEDI6012 in an amount of 100-200 mg at about 48 hours±8 hours following the first dose; and administering intravenously to the subject a third dose of the LCAT enzyme MEDI6012 in an amount of 100-150 mg at about 7 to 10 days following the second dose, to treat heart disease or cardiovascular disease and/or the symptoms thereof in the subject. In an embodiment of the method, the first dose of the LCAT enzyme MEDI6012 is 300 mg; the second dose of MEDI6012 is 150 mg; and the third dose of MEDI6012 is 100 mg. In an embodiment of the method, at least the first dose of the LCAT enzyme MEDI6012 is administered to the subject by IV push.

Provided in yet another aspect described herein is a method of increasing endogenous levels of high density lipoprotein-cholesterol (HDL-C) and/or apoplipoprotein A1 (apoA1) in a subject who has heart disease or cardiovascular disease and/or the symptoms thereof, in which the method comprises administering intravenously to the subject a first loading dose of recombinant human LCAT (rhLCAT) enzyme MEDI6012 in an amount of 300 mg by intravenous (IV) push over a time period of about 1-5 minutes; administering intravenously to the subject a second dose of the LCAT enzyme MEDI6012 in an amount of 150 mg at about 48 hours±8 hours following the first dose; and administering intravenously to the subject a third dose of the LCAT enzyme MEDI6012 in an amount of 100 mg at about 7 days following the second dose, to treat heart disease or cardiovascular disease and/or the symptoms thereof to increase endogenous levels of high density lipoprotein-cholesterol (HDL-C) and/or apoplipoprotein A1 (apoA1) in the subject, thereby treating the heart disease or cardiovascular disease and/or the symptoms thereof. In an embodiment of the method, the first and subsequent doses of MEDI6012 are administered to the subject by IV push over a time period of about 1-3 minutes.

In an embodiment of any of the above aspects or any aspect of the methods delineated herein, the subject has acute or chronic heart disease, cardiovascular disease, coronary artery disease (CAD), stable CAD, atherosclerosis, atherosclerotic cardiovascular disease (CVD), stable CVD, unstable CVD, acute coronary syndrome (ACS), heart failure (HF), congestive HF, hospitalized HF, heart failure with reduced ejection fraction (EF), heart failure with preserved EF, ST-elevated myocardial infarction (STEMI), non-STEMI, or a disease, pathology, or condition related to or associated with heart or cardiac disease, familial or acquired, such as stroke, ischemic stroke, myocardial disease, peripheral artery disease, myocardial infarction, ischemic cardiomyopathy, non-ischemic cardiomyopathy, chemotherapy-induced cardiomyopathy, cerebrovascular disease, acute or chronic renal disease, and/or symptoms thereof. In a particular embodiment, the treated subject has stable CVD.

In an embodiment of any of the above aspects or any aspect of the methods delineated herein, the dose (or first dose) of the isolated and purified LCAT enzyme or MEDI6012 is administered to the subject immediately, e.g., within about or equal to 1-5 minutes, or within about or equal to 1-3 minutes, upon presentation of the subject to a medical facility (hospital, clinic, urgent care center, medical practitioner's office and the like).

In an embodiment of any of the above aspects or any aspect of the methods delineated herein, the administration of the isolated and purified LCAT enzyme or MEDI6012 increases endogenous levels of high density lipoprotein-cholesterol (HDL-C) and/or apolipoprotein A1 (apoA1) in the subject following administration. In other embodiments of the methods, the administration of LCAT decreases, or does not alter or increase, the levels of apolipoprotein B (apoB) in the subject following administration. In another embodiment of the methods of any of the foregoing aspects, the administration of the isolated and purified LCAT enzyme or MEDI6012 does not increase endogenous low density lipoprotein-cholesterol (LDL-C) and produces little to no increase in very large HDL (VL-HDL) particles and very, very large HDL (VVL-HDL) particles.

In an embodiment of any of the above aspects or any aspect of the methods delineated herein, the administration of the isolated and purified LCAT or MEDI6012 affords a myocardio protective effect by preventing myocardial cell death and a reduction in atherosclerotic plaque in the subject. In an embodiment of any of the above aspects or any aspect of the methods delineated herein, the administration of the isolated and purified LCAT or MEDI6012 affords a myocardio protective effect by preventing myocardial fibrosis and hypertrophy.

In an embodiment of any of the above aspects or any aspect of the methods delineated herein, the subject undergoing treatment is taking a statin drug.

In an embodiment of any of the above aspects or any aspect of the methods delineated herein, the isolated and purified LCAT enzyme, rhLCAT, or MEDI6012 is administered to the subject in combination with one or more therapeutic drugs, medicines, or compounds. In an embodiment, the one or more therapeutic drug, medicine, or compound is a statin drug, a proprotein convertase subtilisin/kexin type 9 (PCSK9) enzyme inhibitor (PCSK9i), or other cholesterol-lowering agent. In particular embodiments of the method, the statin drug, PCSK9 inhibitor, or other cholesterol-lowering agent is selected from atorvastatin (LIPITOR), fluvastatin (LESCOL), lovastatin (MEVACOR, ALTOPREV), pitavastatin (LIVALO), pravastatin (PRAVACHOL), rosuvastatin (CRESTOR), simvastatin (ZOCOR), evolocumab (REPATHA®), or alirocumab (PRALUENT®). In embodiments of the method, LCAT or MEDI6012 is administered to the subject before, at the same time as, after, or at a different time than the administration of the one or more therapeutic drugs, medicines, or compounds.

Provided in yet another aspect described herein is a method of providing cardiotherapeutic, myocardioprotective and anti-atherogenic effects in a subject, in which the method comprises administering to a subject having heart disease, cardiovascular disease and/or a symptom thereof a parenteral dose of an isolated and purified LCAT enzyme at a dose of 80-500 mg, wherein endogenous HDL-C levels increase in the subject within about 1 minute to at least 6 hours and/or endogenous apoA1 levels increase within about 12-24 hours following administration of LCAT to the subject. In an embodiment of the method, the administration of LCAT provides cardiotherapeutic, myocardioprotective and anti-atherogenic effects by preventing myocardial fibrosis and hypertrophy in the subject. In a particular embodiment of the method, LCAT is administered at a dose of 300 mg. In another embodiment, the method further comprises administering to the subject a second dose of LCAT in an amount of 125-250 mg at about 48 hours±8 hours following the parenteral dose. In a particular embodiment, the second dose of LCAT is administered to the subject in an amount of 150 mg. In another embodiment of the method, the endogenous levels of HDL-C and/or apoA1 remain elevated for at least 14 days following the administration of LCAT. In an embodiment of the method, LCAT is intravenously administered to the subject. In a particular embodiment of the method, the parenteral dose of LCAT is administered to the subject by IV push over a time period of about 1-3 minutes. In embodiments of the method, the subject has acute or chronic heart disease, cardiovascular disease, coronary artery disease (CAD), stable CAD, atherosclerosis, atherosclerotic cardiovascular disease (CVD), stable CVD, unstable CVD, acute coronary syndrome (ACS), heart failure (HF), congestive HF, hospitalized HF, heart failure with reduced ejection fraction (EF), heart failure with preserved EF, ST-elevated myocardial infarction (STEMI), non-STEMI, or a disease, pathology, or condition related to or associated with heart or cardiac disease, familial or acquired, such as stroke, ischemic stroke, myocardial disease, peripheral artery disease, myocardial infarction, ischemic cardiomyopathy, non-ischemic cardiomyopathy, chemotherapy-induced cardiomyopathy, cerebrovascular disease, acute or chronic renal disease, and/or symptoms thereof. In embodiments, the method further provides cardiotherapeutic, cardioprotective and anti-atherogenic effects and myocardioprotective effects by preventing myocardial fibrosis and hypertrophy. In an embodiment of the method, the isolated and purified LCAT is recombinant human LCAT (rhLCAT). In a particular embodiment, the rhLCAT is MEDI6012 (SEQ ID NO: 2).

In an embodiment of any of the above aspects or any aspect of the methods delineated herein, endogenous HDL-C and/or apoA1 levels are increased in a sample obtained from the subject (e.g., serum or plasma) within about 90 minutes to 6 hours following administration of LCAT or MEDI6012. In an embodiment, the endogenous HDL-C and/or apoA1 levels increase by approximately 50% in the subject's serum or plasma within about 90 minutes and/or endogenous HDL-C levels increase at least 90% in the subject's serum or plasma by about 6 hours following administration of LCAT or MEDI6012, relative to control levels. In yet another embodiment, apoA1 levels remain elevated for at least 7 days in the subject (as detected in the serum or plasma of the subject) following the administration of LCAT or MEDI6012.

In an embodiment of any of the above aspects or any aspect of the methods delineated herein, the administration of LCAT or MEDI6012 protects the subject against developing or worsening of one or more of stroke, ischemic stroke, myocardial damage, kidney damage, liver damage, or increased infarct size. In an embodiment of any of the above aspects or any aspect of the methods delineated herein, the isolated and purified LCAT is recombinant human LCAT (rhLCAT) enzyme or MEDI6012.

Provided in yet another aspect described herein is a method of increasing endogenous concentrations of high density lipoprotein-cholesterol (HDL-C) and/or apolipoprotein A1 (apoA1) and not increasing (i.e., decreasing or causing little no increase in) endogenous concentrations of apolipoprotein B (apoB) in a subject who has or who is at risk of heart disease, heart-related disease, coronary artery disease and/or symptoms thereof, in which the method comprises administering intravenously to the subject a first dose of isolated and purified lecithin-cholesterol acyltransferase (LCAT), recombinant human lecithin-cholesterol acyltransferase (rhLCAT), or MEDI6012 in an amount of from 40-500 mg upon presentation of the subject to a medical professional or medical facility; and administering intravenously to the subject a second dose and at least one subsequent maintenance dose of LCAT, rhLCAT or MEDI6012 in an amount of 40-300 mg at predetermined intervals following the first dose. In an embodiment of the method, the first dose of LCAT, rhLCAT, or MEDI6012 is administered to the subject in an amount selected from 24 mg, 40 mg, 120 mg, 150 mg, or 300 mg. In a particular embodiment of the method, the first dose of LCAT, rhLCAT, or MEDI6012 is administered to the subject in an amount of 300 mg. In another particular embodiment of the method, the first dose of LCAT, rhLCAT, or MEDI6012 is administered to the subject by IV push over a time period of about 1-3 minutes. In another embodiment of the method, the second dose of LCAT, rhLCAT, or MEDI6012 is administered to the subject in an amount selected from 40 mg, 80 mg, 100 mg, 120 mg, or 150 mg. In a particular embodiment of the method, the second dose of LCAT, rhLCAT, or MEDI6012 is administered to the subject in an amount of 150 mg. In an embodiment of the method, the second dose of LCAT, rhLCAT, or MEDI6012 is administered to the subject about 48 hours±8 hours following the first dose. In an embodiment of the method, the at least one subsequent maintenance dose of LCAT, rhLCAT, or MEDI6012 is administered to the subject in an amount selected from 40 mg, 80 mg, 100 mg, 120 mg, or 150 mg following the second dose. In a particular embodiment, the at least one subsequent maintenance dose of LCAT, rhLCAT, or MEDI6012 is administered to the subject in an amount of 100 mg. In another embodiment of the method, the at least one subsequent maintenance dose of LCAT, rhLCAT, or MEDI6012 is administered to the subject about a week following the second dose. In another embodiment of the method, the at least one subsequent maintenance dose of LCAT, rhLCAT, or MEDI6012 is administered to the subject by IV push. In a particular embodiment, MEDI6012 (SEQ ID NO: 2) is administered to the subject. In other embodiments of the method, the subject has or is at risk of having acute or chronic heart disease, cardiovascular disease, coronary artery disease (CAD), stable CAD, atherosclerosis, atherosclerotic cardiovascular disease (CVD), stable CVD, unstable CVD, acute coronary syndrome (ACS), heart failure (HF), congestive HF, hospitalized HF, heart failure with reduced ejection fraction (EF), heart failure with preserved EF, ST-elevated myocardial infarction (STEMI), non-STEMI, or a disease, pathology, or condition related to or associated with heart or cardiac disease, familial or acquired, such as stroke, ischemic stroke, myocardial disease, peripheral artery disease, myocardial infarction, ischemic cardiomyopathy, non-ischemic cardiomyopathy, chemotherapy-induced cardiomyopathy, cerebrovascular disease, acute or chronic renal disease, and/or symptoms thereof. In another embodiment of the method, the subject is concurrently receiving statin drug, a PCSK9 inhibitor, or anti-cholesterol medication therapy.

The present disclosure also provides LCAT, including rhLCAT and MEDI6012, for use in treating a subject with heart disease or cardiovascular disease and/or symptoms thereof in accordance with the methods disclosed herein. The present disclosure also provides the use of LCAT, including rhLCAT and MEDI6012, for the manufacture of a medicament for treating a subject with heart disease or cardiovascular disease and/or symptoms thereof in accordance with the methods disclosed herein.

Other features and advantages of the present disclosure will be apparent from the detailed description, and the claims.

### Definitions

Unless defined otherwise, all technical and scientific terms used herein have the meaning commonly understood by a person skilled in the art to which this invention belongs. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

The term “agent” refers to a protein, polypeptide, peptide (or fragment thereof), nucleic acid molecule, small compound, drug, or medicine.

By “ameliorate” is meant to decrease, reduce, diminish, suppress, attenuate, arrest, inhibit, block, or stabilize the development or progression of a disease or condition.

“Lecithin-cholesterol acyltransferase (LCAT)” (also known as phosphatidylcholine-sterol acyltransferase) is an enzyme that plays a role in the extracellular metabolism of plasma lipoproteins and in removing cholesterol from the blood and tissues. Synthesized in the liver and secreted into plasma, the LCAT enzyme catalyzes the production of cholesteryl ester (CE) from free cholesterol and phosphatidylcholine (lecithin) and helps transport cholesterol out of the blood and tissues. Because LCAT is responsible for producing HDL-CE through the esterification of the free cholesterol component of HDL-C, increasing LCAT levels, function, and/or activity also increases the amount of HDL-CE that is available for delivery to tissues. In humans, about 90% of CE in plasma is formed by LCAT, and the reaction mostly occurs on high-density lipoproteins (HDL), called α-LCAT activity, and to a lesser extent on apolipoprotein B (apoB)-containing particles called β-LCAT activity. The esterification of cholesterol by LCAT helps to maintain HDL levels by promoting the maturation of small discoidal forms of HDL (preβ-HDL and α₄-HDL) into larger spherical forms of HDL (α₁₋₃-HDL), which have longer half-lives. In humans, most HDL-CEs are eventually transferred in exchange for triglycerides to very low-density lipoproteins (VLDL), intermediate-density lipoproteins (IDL), and low-density lipoproteins (LDL) by cholesteryl ester transfer protein (CETP). This process results in a form of cholesterol that is more efficiently carried by lipoproteins, which transport cholesterol back to the liver where the cholesterol is redistributed to other tissues or removed from the body. As used herein, “LCAT enzyme” refers to an isolated and purified LCAT enzyme, such as recombinant human LCAT (rhLCAT) enzyme or MEDI6012.

By “human LCAT polypeptide” is meant a polypeptide or fragment thereof having at least about 85%, about 90%, about 95%, about 96%, about 97%, about 98% or about 99% amino acid sequence identity to UniProtKB Accession No. P04180-1 or to NCBI Reference Sequence: NP_000220.1 and having LCAT enzymatic activity and/or function. (SEQ ID NO: 1 below).

```
(SEQ ID NO: 1)
                10 20 30 40
MGPPGSPWQW VTLLLGLLLP PAAPFWLLNV LFPPHTTPKA
                50 60 70 80
ELSNHTRPVI LVPGCLGNQL EAKLDKPDVV NWMCYRKTED
                90 100 110 120
FFTIWLDLNM FLPLGVDCWI DNTRVVYNRS SGLVSNAPGV
              130 140 150 160
QIRVPGFGKT YSVEYLDSSK LAGYLHTLVQ NLVNNGYVRD
              170 180 190 200
ETVRAAPYDW RLEPGQQEEY YRKLAGLVEE MHAAYGKPVF
              210 220 230 240
LIGHSLGCLH LLYFLLRQPQ AWKDRFIDGF ISLGAPWGGS
              250 260 270 280
IKPMLVLASG DNQGIPIMSS IKLKEEQRIT TTSPWMFPSR
              290 300 310 320
MAWPEDHVFI STPSFNYTGR DFQRFFADLH FEEGWYMWLQ
              330 340 350 360
SRDLLAGLPA PGVEVYCLYG VGLPTPRTYI YDHGFPYTDP
              370 380 390 400
VGVLYEDGDD TVATRSTELC GLWQGRQPQP VHLLPLHGIQ
              410 420 430 440
HLNMVFSNLT LEHINAILLG AYRQGPPASP TASPEPPPPE
```

By “MEDI6012 (recombinant human LCAT (rhLCAT) polypeptide)” is meant a polypeptide of 416 amino acids, as shown in SEQ ID NO: 2 below, or a fragment thereof, having LCAT enzymatic activity and/or function, or a polypeptide or a fragment thereof having at least about 85%, about 90%, about 95%, about 96%, about 97%, about 98% or about 99% amino acid sequence identity to the amino acid sequence of SEQ ID NO: 2 and having LCAT enzymatic activity and/or function.

(SEQ ID NO: 2)
FWLLNVLFPP HTTPKAELSN HTRPVILVPG CLGNQLEAKL
DKPDVVNWMC YRKTEDFFTI WLDLNMFLPL GVDCWIDNTR
VVYNRSSGLV SNAPGVQIRV PGFGKTYSVE YLDSSKLAGY
LHTLVQNLVN NGYVRDETVR AAPYDWRLEP GQQEEYYRKL
AGLVEEMHAA YGKPVFLIGH SLGCLHLLYF LLRQPQAWKD
RFIDGFISLG APWGGSIKPM LVLASGDNQG IPIMSSIKLK
EEQRITTTSP WMFPSRMAWP EDHVFISTPS FNYTGRDFQR
FFADLHFEEG WYMWLQSRDL LAGLPAPGVE VYCLYGVGLP
TPRTYIYDHG FPYTDPVGVL YEDGDDTVAT RSTELCGLWQ
GRQPQPVHLL PLHGIQHLNM VFSNLTLEHI NAILLGAYRQ
GPPASPTASP EPPPPE

The polynucleotide coding sequence for human LCAT polypeptide is presented below (1323 nucleotides (nts)). A polynucleotide or fragment thereof having at least about 85%, about 90%, about 95%, about 96%, about 97%, about 98% or about 99% nucleotide sequence identity to the human LCAT nucleic acid sequence of NCBI CCDS Accession No. 10854.1 (SEQ ID NO: 3 below) is encompassed by the disclosure.

(SEQ ID NO: 3)
ATGGGGCCGCCCGGCTCCCCATGGCAGTGGGTGACGCTGCTGCTGGG
GCTGCTGCTCCCTCCTGCCGCCCCCTTCTGGCTCCTCAATGTGCTCT
TCCCCCCGCACACCACGCCCAAGGCTGAGCTCAGTAACCACACACGG
CCCGTCATCCTCGTGCCCGGCTGCCTGGGGAATCAGCTAGAAGCCAA
GCTGGACAAACCAGATGTGGTGAACTGGATGTGCTACCGCAAGACAG
AGGACTTCTTCACCATCTGGCTGGATCTCAACATGTTCCTACCCCTT
GGGGTAGACTGCTGGATCGATAACACCAGGGTTGTCTACAACCGGAG
CTCTGGGCTCGTGTCCAACGCCCCTGGTGTCCAGATCCGCGTCCCTG
GCTTTGGCAAGACCTACTCTGTGGAGTACCTGGACAGCAGCAAGCTG
GCAGGGTACCTGCACACACTGGTGCAGAACCTGGTCAACAATGGCTA
CGTGCGGGACGAGACTGTGCGCGCCGCCCCCTATGACTGGCGGCTGG
AGCCCGGCCAGCAGGAGGAGTACTACCGCAAGCTCGCAGGGCTGGTG
GAGGAGATGCACGCTGCCTATGGGAAGCCTGTCTTCCTCATTGGCCA
CAGCCTCGGCTGTCTACACTTGCTCTATTTCCTGCTGCGCCAGCCCC
AGGCCTGGAAGGACCGCTTTATTGATGGCTTCATCTCTCTTGGGGCT
CCCTGGGGTGGCTCCATCAAGCCCATGCTGGTCTTGGCCTCAGGTGA
CAACCAGGGCATCCCCATCATGTCCAGCATCAAGCTGAAAGAGGAGC
AGCGCATAACCACCACCTCCCCCTGGATGTTTCCCTCTCGCATGGCG
TGGCCTGAGGACCACGTGTTCATTTCCACACCCAGCTTCAACTACAC
AGGCCGTGACTTCCAACGCTTCTTTGCAGACCTGCACTTTGAGGAAG
GCTGGTACATGTGGCTGCAGTCACGTGACCTCCTGGCAGGACTCCCA
GCACCTGGTGTGGAAGTATACTGTCTTTACGGCGTGGGCCTGCCCAC
GCCCCGCACCTACATCTACGACCACGGCTTCCCCTACACGGACCCTG
TGGGTGTGCTCTATGAGGATGGTGATGACACGGTGGCGACCCGCAGC
ACCGAGCTCTGTGGCCTGTGGCAGGGCCGCCAGCCACAGCCTGTGCA
CCTGCTGCCCCTGCACGGGATACAGCATCTCAACATGGTCTTCAGCA
ACCTGACCCTGGAGCACATCAATGCCATCCTGCTGGGTGCCTACCGC
CAGGGTCCCCCTGCATCCCCGACTGCCAGCCCAGAGCCCCCGCCTCC
TGAATAA.

The cloning and sequencing of a human LCAT cDNA is reported in J. McLean et al., 1986, Proc. Nat'l. Acad. Sci. USA, 83:2335-2339; the complete gene sequence for human LCAT is reported in J. McLean et al., 1986, Nucl. Acids Res., 14:9397-9406.

MEDI6012 (formerly called ACP501) is an isolated and purified recombinant human LCAT (rhLCAT) enzyme. MEDI6012 (rhLCAT) is an approximately 60 kilodalton, glycosylated, single-chain protein consisting of 416 amino acids and is produced in and isolated and purified from Chinese hamster ovary (CHO) cell culture. In an embodiment, MEDI6012 is used in methods of treatment to reduce the risk of ischemic events as adjunct to the standard of care in patients with acute coronary syndrome (ACS) and to reduce the risk of cardiovascular (CV) death and heart failure (HF) hospitalization in patients with high risk myocardial infarction. MEDI6012 and ACP501 have the identical amino acid sequence and are therefore considered the same molecular entity. MEDI6012 is manufactured to provide greater enzymatic activity on a per-mg of protein basis and increased product- and process-related purity for MEDI6012 relative to the former ACP501.

A “biomarker” or “marker” as used herein generally refers to a protein, nucleic acid molecule, clinical indicator, or other analyte that is associated with a disease. In one embodiment, a marker is differentially present in a biological sample obtained from a subject having a disease, e.g., heart disease, cardiovascular disease, or coronary artery disease, relative to the level present in a control sample or reference. In an embodiment, a marker is a pharmacodynamic (PD) marker that is assessed in a subject who has been treated with a drug, e.g., MEDI6012, e.g., by measuring or quantifying its level in a sample (e.g., blood, plasma, or serum) obtained from the subject, compared with a control, such as the level of the same PD marker in the sample from a subject who has been treated with placebo. PD biomarkers are markers, targets, or determinants that can be quantitatively and/or qualitatively assessed to determine whether a given agent, e.g., a drug, compound, or medicine, is producing one or more pharmacological or physiological effects. PD biomarkers are indicators of a drug's direct or indirect effect (activity) on a target in an organism and may be useful in examining the association or link among a drug dose and/or drug regimen, target effect and a biological response.

Cardiac or heart disease refers to any type of disorder that affects the heart, including heart muscle tissue and cells (called myocardiocytes). Heart disease encompasses several disorders or conditions including myocardial infarction (known as heart attack or coronary thrombosis), in which blood flow is interrupted resulting in a lack of oxygen that damages or destroys a portion of heart muscle. Coronary artery disease involves disease or damage to the coronary arteries that supply the heart with nutrients, oxygen and blood, usually resulting from plaque (cholesterol-containing) deposits and accumulation that narrow the artery openings and decrease flow to the heart/heart muscle.

Cardiovascular diseases (CVDs) are a group of disorders of both the heart and blood vessels, which include coronary heart disease (disease of the blood vessels supplying the heart muscle); cerebrovascular disease (disease of the blood vessels supplying the brain); peripheral arterial disease (disease of blood vessels supplying the arms and legs); rheumatic heart disease (damage to the heart muscle and heart valves from rheumatic fever, caused by streptococcal bacteria); congenital heart disease (malformations of heart structure existing at birth); deep vein thrombosis and pulmonary embolism (blood clots in the leg veins, which can dislodge and move to the heart and lungs). Heart attacks and strokes are acute events with chronic consequence/sequelae and are mainly caused by a blockage that prevents blood from flowing to the heart or brain. The most common reason for this is a build-up of fatty deposits on the inner walls of the blood vessels that supply blood to the heart or brain. Strokes can also be caused by bleeding from a blood vessel in the brain or from blood clots. The causes of heart attacks and strokes are usually the presence of a combination of risk factors, such as tobacco use, unhealthy diet and obesity, physical inactivity and harmful use of alcohol, hypertension, diabetes, hyperlipidemia, and genetic predisposition.

“HDL” is an acronym for “high density lipoprotein”. Reconstituted HDL (rHDL) refers to a complex of apolipoprotein A1 (apoA1), phospholipid (e.g., lecithin) and cholesteryl ester (CE), or a complex of apoA1, phospholipid (e.g., lecithin), cholesterol and cholesteryl ester (CE). HDL in complex with apoA1, phospholipid and CE is also referred to as an HDL particle. Phospholipids that may be used in producing rHDL include phosphatidylcholine, sphingomyelin, phosphatidylethanolamine, phosphatidylserine, phosphatidylinositol, phosphatidylglycerol, cardiolipin, or mixtures thereof. Native HDL is isolated from plasma or serum and refers to particles that contain HDL, proteins (such as apoA1), cholesterol and cholesteryl ester. “HDL-C” refers to HDL which may contain both esterified and unesterified cholesterol (“C” represents total cholesterol comprising both cholesterol (C) and cholesteryl ester (CE)). “HDL-CE” refers to the cholesteryl ester component of HDL. ApoA1 is the primary protein associated with HDL particles and plays a role in reverse cholesterol transport. There are a variable number of apoA1 proteins per HDL particle, and the number of apoA1 proteins and the amount of cholesterol contained in HDL particles is also variable.

As used herein, the terms “determining”, “assessing”, “assaying”, “measuring” and “detecting”, and “identifying” refer to both quantitative and qualitative determinations, and as such, the term “determining” is used interchangeably herein with “assaying,” “measuring,” and the like. Where a quantitative determination is intended, the phrase “determining an amount” of an analyte, substance, protein, and the like is used. Where a qualitative and/or quantitative determination is intended, the phrase “determining a level” of an analyte or “detecting” an analyte is used.

By “disease” is meant any condition or disorder that damages, interferes with or dysregulates the normal function of a cell, tissue, or organ. In particular, cardiac disease is also called heart disease. Diseases of and associated with the heart and coronary or peripheral arteries as referred to herein include, by way of example, acute or chronic heart disease, cardiovascular disease, coronary artery disease (CAD), stable CAD, atherosclerosis, atherosclerotic cardiovascular disease (CVD), stable CVD, unstable CVD, acute coronary syndrome (ACS), heart failure (HF), congestive HF, hospitalized HF, heart failure with reduced ejection fraction (EF), heart failure with preserved EF, ST-elevated myocardial infarction (STEMI), non-STEMI, or a disease, pathology, or condition related to or associated with heart or cardiac disease, familial or acquired, such as stroke, ischemic stroke, myocardial disease, peripheral artery disease, myocardial infarction, ischemic cardiomyopathy, non-ischemic cardiomyopathy, chemotherapy-induced cardiomyopathy, cerebrovascular disease, acute or chronic renal disease, and/or symptoms thereof. Such diseases, conditions and/or the symptoms thereof may be acute or chronic in a subject and are not intended to be limiting.

The terms “isolated,” “purified” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide is purified, as used herein, if it is substantially free of cellular material, viral material, or culture medium when produced by recombinant DNA techniques, or chemical precursors, or other chemicals when chemically synthesized. Purity and homogeneity are typically determined using analytical chemistry techniques, for example, polyacrylamide gel electrophoresis, high performance liquid chromatography (HPLC), mass spectrometry analysis, etc. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid (e.g., a DNA) that is free of the genes which, in the naturally-occurring genome of the organism from which the nucleic acid molecule is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding one or more additional polypeptide sequences.

By an “isolated polypeptide” is meant a polypeptide of the disclosure, such as isolated LCAT or recombinant human LCAT enzyme, that has been separated from components that naturally accompany it, or from components that are present during an isolation or purification process. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the disclosure. An isolated polypeptide of the disclosure may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

The term “dose” refers to a measured quantity, amount, or concentration of a therapeutic agent, such as a drug, medicine, compound, e.g., a small molecule or biologic, that is administered (without limitation to route of administration) to a subject or patient who has a need for the agent, such as for treatment or therapy benefit.

A “dose or dosing regimen” as used herein refers to the dose or dosage amount (of LCAT, such as rhLCAT or MEDI6012) administered to a subject at a certain dosing frequency (number of times a drug is administered) for a given treatment period (length of treatment), e.g., days, weeks, months, years, etc.

A “loading dose” as used herein refers to a comparatively large amount or concentration (such as a bolus dose) of a drug, e.g., rhLCAT (MEDI6012), given at the beginning of a course of treatment to provide for an initial effect, exposure, or impact of a drug in a subject, especially a drug which has slow clearance from the body (a long systemic half-life), before giving a lower or maintenance dose of the drug, which maintains the amount or concentration of the drug in the body at an appropriate therapeutic level. In general, providing a loading dose accelerates the time needed for a therapeutic level of the drug to be reached in the body. More specifically, in the methods described herein in which LCAT (e.g., rhLCAT or MEDI6012) is administered in the described doses, the loading dose accelerates the time in which a desired PD effect is attained, such as, e.g., an increase in levels or amounts of HDL-C and/or apoA1. Calculation of the loading dose generally involves four variables, namely, Cₚ, the desired peak concentration of the drug; V_{d}, the volume of distribution of drug in the body; F, the bioavailability of the drug; and S, the fraction of the drug (or drug salt form) that is active in the body. The loading dose may be calculated as:

$$\frac{{C}_{p} {V}_{d}}{FS}$$

For a drug administered intravenously, the bioavailability F will equal 1, as the drug is introduced directly into the bloodstream.

A “maintenance dose” refers to a dose of a drug or medicament, such as isolated and purified LCAT (e.g., rhLCAT or MEDI6012 described herein), which maintains the amount or concentration of the drug in the body at an appropriate therapeutic level. A maintenance dose of a drug or medicament is frequently administered at a predetermined time and/or at repeated, predetermined time intervals (e.g., weekly, monthly, and the like) following the administration of an initial dose (e.g., loading dose) or previous dose of the drug or medicament. A maintenance dose of the drug or medicament is typically lower or significantly lower than a loading dose. A maintenance dose of the drug or medicament may be given to a subject over a prolonged time period following an initial or loading dose, or a previous dose.

Reverse cholesterol transport (RCT) is a multi-step process resulting in the net movement of cholesterol from peripheral tissues back to the liver via the plasma compartment for reuse or excretion in the bile. Cellular cholesterol efflux is mediated by high density lipoprotein (HDL), acting in conjunction with LCAT. The major steps in the RCT pathway are the efflux of free cholesterol from cells and binding by pre-beta HDL, esterification of HDL-bound cholesterol by lecithin cholesterol acyl transferase (LCAT), cholesteryl ester transfer protein (CETP) mediated exchange of cholesteryl ester and triglycerides between HDL and apo B-containing particles, and hepatic lipase (HL) mediated uptake of cholesterol and triglycerides by the liver. Thus, cholesteryl ester accumulating in HDL can follow a number of different fates, such as uptake in the liver in HDL-containing apolipoprotein (particle uptake) by low density lipoprotein (LDL) receptors, selective uptake of HDL cholesteryl ester in liver or other tissues involving scavenger receptor B1 (SRB1), or transfer to triglyceride-rich lipoproteins as a result of the activity of cholesteryl ester transfer protein, with subsequent uptake of triglyceride-rich lipoprotein remnants in the liver.

By “reference” or “control” is meant a standard of comparison, such as a placebo.

By “responsive” in the context of therapy is meant susceptible to treatment.

By “biological sample” or “sample” is meant any liquid, cell, or tissue obtained from a subject. In some embodiments, the biological sample is blood, serum, plasma, cerebrospinal fluid, bronchoalveolar lavage, sputum, tears, saliva, urine, semen, feces, etc. Cell or tissue samples may be further processes in a suitable buffer to produce a homogenate or suspension in which the intracellular components of cells and tissue are provided. In certain embodiments, a blood, plasma, or serum sample is utilized for biomarker and marker (e.g., PD marker) detection and quantification.

By “subject” is meant a mammal, including, but not limited to, a human, such as a human patient, a non-human primate, or a non-human mammal, such as a bovine, equine, canine, ovine, or feline animal. In an embodiment, the subject is a human. In an embodiment, a subject is a human patient who has, is at risk for, or who has and is undergoing treatment for a heart (cardiac) condition or disease, or cardiovascular disease or syndrome and/or symptoms thereof. In an embodiment, the subject with a heart condition may have atherosclerosis or coronary artery disease.

Ranges provided herein are understood to be shorthand for all of the values within the range, inclusive of the first and last stated values. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

A “pharmaceutical composition” or “formulation” refers to a composition (a physiologically acceptable composition) suitable for pharmaceutical use in a subject, such as an animal or a mammal, including humans A pharmaceutical composition comprises a therapeutically or prophylactically effective amount of MEDI6012 and a pharmaceutically acceptable excipient, carrier, vehicle, or diluent. In an embodiment, a pharmaceutical composition encompasses a composition comprising the active ingredient(s) (MEDI6012 or rhLCAT), and the inert ingredient(s) that constitute the carrier, as well as any product that results, directly or indirectly, from combination, complexation or aggregation of any two or more of the ingredients, or from dissociation of one or more of the ingredients, or from other types of reactions or interactions of one or more of the ingredients. In an embodiment, the pharmaceutical composition optionally includes another biologically active agent, compound, drug, or medicine. Accordingly, the pharmaceutical compositions of the present disclosure embrace any composition that is made by admixing rhLCAT or MEDI6012 and a pharmaceutically acceptable excipient, carrier, vehicle, or diluent.

A “pharmaceutically acceptable carrier” refers to any of the standard pharmaceutical carriers, buffers, and the like, such as a phosphate buffered saline solution, optionally another biologically active agent, an aqueous (e.g., 5%) solution of dextrose, and emulsions (e.g., an oil/water or water/oil emulsion). Non-limiting examples of excipients include adjuvants, binders, fillers, diluents, disintegrants, emulsifying agents, wetting agents, lubricants, glidants, sweetening agents, flavoring agents, and coloring agents. Suitable pharmaceutical carriers, excipients, vehicles and diluents may be found in Remington's Pharmaceutical Sciences, 19th Ed. (Mack Publishing Co., Easton, 1995 (or updated editions of this reference)). A pharmaceutical carrier suitable for inclusion in a composition or formulation typically depends upon the intended mode of administration of the active agent, e.g., MEDI6012. Illustrative modes of administration include enteral (e.g., oral) or parenteral (e.g., subcutaneous, intramuscular, intravenous or intraperitoneal injection; intravenous infusion, or topical, transdermal, or transmucosal administration).

A “pharmaceutically acceptable salt” refers to a salt that can be formulated into a compound for pharmaceutical use, including, but not limited to, metal salts (e.g., sodium, potassium, magnesium, calcium, etc.) and salts of ammonia or organic phosphate

“Pharmaceutically acceptable,” physiologically acceptable,” or “pharmacologically acceptable” refers to a material that is not biologically, physiological, or otherwise undesirable, i.e., the material may be administered to an individual without causing any undesirable biological effects or without interacting in a deleterious manner with any of the components of the composition in which it is contained or with any components present on or in the body of the individual.

“Physiological conditions” refer to conditions in the body of an animal or mammal, such as a human Physiological conditions include, but are not limited to, body temperature and an aqueous environment of physiologic ionic strength, pH and enzymes. Physiological conditions also encompass conditions in the body of a particular subject which differ from the “normal” conditions present in the majority of subjects, such as normal human body temperature (approximately 37° C.) or normal human blood pH (approximately 7.4).

As used herein, the terms “treat,” treating,” “treatment,” and the like refer to reducing, diminishing, lessening, alleviating, abrogating, or ameliorating a disorder and/or symptoms associated therewith. It will be appreciated that, although not precluded, treating a disorder or condition does not require that the disorder, condition or symptoms associated therewith be completely eliminated. “Treatment” may refer to prophylactic treatment or therapeutic treatment or diagnostic treatment. In certain embodiments, “treatment” refers to administration of a compound or composition to a subject for therapeutic, prophylactic or diagnostic purposes.

In accordance with the described methods, treating or treatment involves the administration of the active ingredient (isolated and purified LCAT, rhLCAT or MEDI6012) as described herein. In an embodiment, isolated and purified LCAT, rhLCAT or MEDI6012 is administered intravenously to a subject in need. As will be appreciated by the skilled practitioner in the art, intravenous administration generally refers to providing or delivering an active ingredient, therapeutic agent, substance, medicament, or drug, such as isolated and purified LCAT, rhLCAT or MEDI6012, and the like, into a vein or blood vessel of a subject to deliver the active ingredient to the systemic circulation of the subject. Intravenous administration may comprise intravenous injection or intravenous infusion into a vein or vessel, e.g., by means of a syringe and needle or catheter. Intravenous injection or infusion may involve the use of plastic tubing and an infusion bag (e.g., an infusion set), such that the active ingredient is delivered through tubing into an infusion bag, and then from the infusion bag into the subject, such as through a catheter and/or a port placed in the subject's body, at a rate of flow that is conventionally and practically determined by a medical practitioner. Intravenous injection or infusion may be carried out with the use of a pump or via a drip. By way of example and without limitation, the administration of active ingredient or medication, such as isolated and purified LCAT, rhLCAT or MEDI6012, by intravenous infusion to a subject may occur over a period of time such as, for example, about 30 minutes to 1 hour or longer, or over about 1 hour.

In an embodiment, intravenous administration may comprise an IV push, which is understood to be delivery (e.g., by injection through a syringe) of active ingredient or medication, such as isolated and purified LCAT, rhLCAT or MEDI6012, into a subject's vein or blood vessel. An IV push may be delivered through an intravenous line, needle, or catheter. In a particular embodiment, an IV push refers to an intravenous injection or infusion of isolated and purified LCAT, rhLCAT or MEDI6012 (drug or medication) which is typically manually delivered to a subject via syringe over a relatively short time period, for example and without limitation, a time period of about or equal to 30 seconds to 3 minutes, or a time period of about or equal to 1-10 minutes, or a time period of about or equal to 1-5 minutes, or a time period of about or equal to 1-3 minutes, or a time period of about or equal to 1-2 minutes, or a time period of about or equal to 1 minute. An IV push is typically administered to a subject via a syringe. An IV push may be delivered through a syringe into a short or long IV line into a vein or vessel of a subject. In a particular embodiment, isolated and purified LCAT, rhLCAT or MEDI6012 is administered to a subject by IV push over a time period of about or equal to 1-3 minutes.

“Prophylactic treatment” (such as a preventive or protective treatment) is a treatment administered to a subject who does not exhibit signs of a disease, or who exhibits only early signs of the disease, or who is at risk for having a disease, for the purpose of reducing, decreasing, alleviating, or eliminating the risk of developing a disease, pathology, or condition or a more serious or severe form of the disease or pathology, or condition. The rhLCAT or MEDI6012 compound or compositions thereof of the disclosure may be given as a prophylactic or protective treatment to reduce the likelihood of a subject developing a disease, pathology, or condition or to minimize the severity of the disease, pathology, or condition if it develops in the subject.

A “therapeutic” treatment is a treatment administered to a subject who exhibits signs or symptoms of a disease or pathology for the purpose of reducing, diminishing, alleviating, or eliminating the signs or symptoms. The signs or symptoms of disease or pathology may be, without limitation, biochemical, behavioral, cellular, phenotypic, genotypic, histological, functional, physical, subjective, or objective. Recombinant human LCAT (rhLCAT) or MEDI6012 of the disclosure may also be given as a therapeutic treatment or for diagnosis.

As used herein, a therapeutic that “prevents” a disorder or condition refers to a compound or material that, in a statistical sample, reduces the occurrence of the disorder or condition in the treated sample relative to an untreated control or reference sample, or delays the onset of, or reduces the severity of one or more symptoms of the disorder or condition relative to an untreated reference or control sample. In an embodiment, MEDI6012 is a preventative therapeutic agent in the methods described herein.

The term “effective amount” refers to a dosage sufficient to produce a desired result (e.g., reduction, abatement, elimination, or amelioration of symptoms) related to a health condition, pathology, or disease of a subject or for a diagnostic purpose. The desired result may comprise a subjective or objective improvement in a subject to whom a dose or dosage is administered. “Therapeutically effective amount” refers to that amount of an agent effective to produce the intended beneficial effect on health. It will be understood that the specific dose level and frequency of dosage for any particular patient may depend upon a variety of factors, including the activity of the specific compound employed; the bioavailability, metabolic stability, rate of excretion and length of action of that compound; the mode and time of administration of the compound; the age, body weight, general health, sex, and diet of the patient; and the severity of the patient's particular condition.

The terms “protein”, “peptide” and “polypeptide” refer to chain of amino acids, regardless of length or post-translational modification (for example, glycosylation or phosphorylation). Thus, the terms can be used interchangeably herein to refer to a polymer of amino acid residues. The terms also apply to amino acid polymers in which one or more amino acid residues is an artificial chemical mimetic of a corresponding naturally occurring amino acid. Thus, the term “polypeptide” includes full-length, naturally occurring proteins, as well as recombinantly or synthetically produced polypeptides that correspond to a full-length naturally occurring protein or to particular domains or portions of a naturally occurring protein. The term also encompasses mature proteins which have an added amino-terminal methionine to facilitate expression in prokaryotic cells. Polypeptides can be chemically synthesized or synthesized by recombinant DNA methods; or, they can be purified from tissues in which they are naturally expressed, according to standard biochemical methods of purification. “Functional polypeptides” possess one or more of the biological functions or activities of a given protein or polypeptide, e.g., the LCAT enzymatic protein. Functional polypeptides may contain a primary amino acid sequence that has been modified from that considered to be the standard sequence of the human LCAT protein. Preferably, such modifications are conservative amino acid substitutions that do not alter or substantially alter the normal function or activity of the protein. A polypeptide fragment, portion, or segment refers to a stretch of amino acid residues of at least about 6 contiguous amino acids from a particular sequence, more typically at least about 10-12 contiguous amino acids.

Nucleic acid molecules (polynucleotides), which encode polypeptides such as LCAT of the present disclosure, include any nucleic acid molecule that encodes the disclosed polypeptide, e.g., human LCAT, or a fragment thereof. Such nucleic acid molecules need not be 100% identical to an endogenous nucleic acid sequence, but will typically exhibit substantial identity. Polynucleotides having “substantial identity” to an endogenous sequence are typically capable of hybridizing with at least one strand of a double-stranded nucleic acid molecule. Polynucleotides having “substantial identity” to an endogenous sequence are typically capable of hybridizing with at least one strand of a double-stranded nucleic acid molecule. By “hybridize” is meant pairing to form a double-stranded molecule between complementary polynucleotide sequences (e.g., a gene), or portions thereof, under various conditions of stringency. (See, e.g., Wahl, G. M. and S. L. Berger, 1987, Methods Enzymol., 152:399; Kimmel, A. R., 1987, Methods Enzymol., 152:507).

Genomic DNA encoding human LCAT of 416 amino acids has been isolated. (See, e.g., U.S. Pat. No. 6,635,614). The nucleotide and deduced amino acid sequence of an LCAT from mouse is described in CH. Warden et al., 1989, J. Biol Chem., 264:21573-81. A mammalian LCAT (particularly, human LCAT), or an enzymatically active allelic variation thereof, may be useful in the described methods, as are other variants, including fragments of the enzyme that possess the enzymatic activity of LCAT. An “allelic variation” in the context of a polynucleotide or a gene is an alternative form (allele) of a gene that exists in more than one form in the population. At the polypeptide level, “allelic variants” generally differ from one another by only one, or at most, a few amino acid substitutions. A “species variation” of a polynucleotide or a polypeptide is one in which the variation is naturally occurring among different species of an organism.

By way of nonlimiting example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a particular embodiment, hybridization occurs at 30° C. in 750 mM NaCl, 75 mM trisodium citrate, and 1% SDS. In another particular embodiment, hybridization occurs at 37° C. in 500 mM NaCl, 50 mM trisodium citrate, 1% SDS, 35% formamide, and 100 μg/ml denatured salmon sperm DNA (ssDNA). In another particular embodiment, hybridization occurs at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will be less than about 30 mM NaCl and 3 mM trisodium citrate, and, in particular, less than about 15 mM NaCl and 1.5 mM trisodium citrate. Stringent temperature conditions for the wash steps will ordinarily include a temperature of at least about 25° C., or at least about 42° C., or at least about 68° C. In a particular embodiment, wash steps will occur at 25° C. in 30 mM NaCl, 3 mM trisodium citrate, and 0.1% SDS. In another particular embodiment, wash steps will occur at 42 C in 15 mM NaCl, 1.5 mM trisodium citrate, and 0.1% SDS. In another particular embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science, 196:180, 1977); Grunstein and Hogness (Proc. Natl. Acad. Sci., USA, 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence or nucleic acid sequence. Such a sequence may be at least 60%, or at least 80% or 85%, or at least 90%, 95%, or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following amino acid groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

In this disclosure, “comprises,” “comprising,” “containing” and “having” and the like can have the meaning ascribed to them in U.S. patent law and can mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

Unless specifically stated or obvious from its context, the term “or” as used herein is understood to be inclusive. Unless specifically stated or obvious from context, the terms “a”, “an”, and “the” as used herein are understood to be singular or plural.

Unless specifically stated or obvious from context, as used herein, the term “about” is understood as within a range of normal tolerance in the art, for example within 2 standard deviations of the mean. The term “about” is understood to refer to within 5%, 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

Any compositions or methods provided herein can be combined with one or more of any of the other compositions and methods provided herein.

### BRIEF DESCRIPTION OF THE DRAWINGS/FIGURES

FIG. 1 presents a schematic overview of the design of the Phase 2a single ascending dose (SAD) clinical study (SAD Clinical Study D5780000002) described in Example 1 herein. The cohorts enrolled in the study represented a population that had stable coronary artery disease (CAD) and that was on statin therapy. The study did not include subjects with recent unstable angina or myocardial infarction (MI), stroke, transient ischemic attack (TIA) or mini-stroke, or vascular intervention. The study further excluded those subjects who had HDL-C levels greater than 60 mg/dL; males and females greater than 75 years of age; LDL-C levels greater than 150 mg/dL (direct measure by a standard laboratory test); and triglyceride (TG) levels greater than 500 mg/dL. In FIG. 1, the term “active” refers to the MEDI6012 rhLCAT enzyme administered to the subjects in each cohort at the indicated doses, 24 mg, 80 mg, 240 mg and 800 mg, delivered by intravenous (IV) administration, and 80 mg and 600 mg delivered by subcutaneous (SC) injection. The term “pbo” refers to placebo administered to subjects in the study.

FIGS. 2A and 2B show graphs of the serum concentration (levels) of LDL-C (direct measure by a standard laboratory test) over time in subjects who received MEDI6012 at a dose of 24, 80, 240 or 800 mg via intravenous (IV) administration compared with placebo as determined in the Phase 2a SAD study described in Example 1 herein. FIG. 2A shows the serum concentration of LDL-C (direct measure by a standard laboratory test) over time in subjects administered single IV doses of MEDI6012. FIG. 2B shows the change from baseline in serum concentration of LDL-C (direct measure by a standard laboratory test) over time in subjects administered single IV doses of MEDI6012 as described for FIG. 2A.

FIGS. 3A and 3B show graphs of the concentration of apolipoprotein B (apoB) in the serum of subjects who received MEDI6012 at a dose of 24, 80, 240 or 800 mg via intravenous (IV) administration over time compared with placebo as determined in the Phase 2a SAD study described in Example 1 herein. FIG. 3A shows the serum concentration of apoB over time in subjects administered single IV doses of MEDI6012 (24, 80, 240 or 800 mg IV doses). FIG. 3B shows the change from baseline in serum concentration of apoB over time in subjects administered single IV doses of MEDI6012 as described for FIG. 3A.

FIGS. 4A-4D show graphs of serum concentrations of HDL-C over time in subjects who received 80 mg or 600 mg doses of MEDI6012 by subcutaneous (SC) administration versus placebo, or in subjects who received 24 mg, 80 mg, 240 mg, or 800 mg doses of MEDI6012 by intravenous (IV) administration versus placebo as determined in the Phase 2a SAD study described in Example 1 herein. FIG. 4A shows the serum concentration of HDL-C over time in subjects administered SC doses of MEDI6012 (80 mg or 600 mg doses) versus placebo control. FIG. 4B shows the change from baseline in serum concentration of HDL-C over time in subjects administered SC doses of MEDI6012 (80 mg or 600 mg doses) versus placebo control. FIG. 4C shows the serum concentration of HDL-C over time in subjects administered IV doses of MEDI6012 (24 mg, 80 mg, 240 mg, or 800 mg doses) versus placebo control. FIG. 4D shows the change from baseline in serum concentration of HDL-C over time in subjects administered IV doses of MEDI6012 (24 mg, 80 mg, 240 mg, or 800 mg doses) versus placebo control.

FIGS. 5A and 5B show graphs of the serum concentration (levels) of LDL-C (direct measure by a standard laboratory test) over time in subjects who received MEDI6012 at a dose of 80 or 600 mg via subcutaneous (SC) administration compared with placebo as determined in the Phase 2a SAD study described in Example 1 herein. FIG. 5A shows the serum concentration of LDL-C (direct measure by a standard laboratory test) over time in subjects administered a single SC dose of MEDI6012 (an 80 or 600 mg SC dose). FIG. 5B shows the change from baseline in serum concentration of LDL-C (direct measure by a standard laboratory test) over time in subjects administered a single SC dose of MEDI6012 as described for FIG. 5A.

FIGS. 6A and 6B show graphs of the concentration of apoB in the serum of subjects who received MEDI6012 at a dose of 80 or 600 mg via subcutaneous (SC) administration over time compared with placebo, as determined in the Phase 2a SAD study described in Example 1 herein. FIG. 6A shows the serum concentration of apoB over time in subjects administered a single SC dose of MEDI6012 (an 80 mg or 600 mg SC dose). FIG. 6B shows the change from baseline in serum concentration of apoB over time in subjects administered a single SC dose of MEDI6012 as described for FIG. 6A

FIGS. 7A-7D show graphs of serum concentrations of apoA1 over time in subjects who received 80 mg or 600 mg doses of MEDI6012 by subcutaneous (SC) administration versus placebo, or in subjects who received 24 mg, 80 mg, 240 mg, or 800 mg doses of MEDI6012 by intravenous (IV) administration versus placebo as determined in the Phase 2a SAD study described in Example 1 herein. FIG. 7A shows the serum concentration of apoA1 over time in subjects administered an SC dose of MEDI6012 (an 80 mg or 600 mg dose). FIG. 7B shows the change from baseline in serum concentration of apoA1 over time in subjects administered an SC dose of MEDI6012 (an 80 mg or 600 mg dose). FIG. 7C shows the serum concentration of apoA1 over time in subjects administered IV doses of MEDI6012 (24 mg, 80 mg, 240 mg, or 800 mg doses) versus placebo control. FIG. 7D shows the change from baseline in serum concentration of apoA1 over time in subjects administered IV doses of MEDI6012 (24 mg, 80 mg, 240 mg, or 800 mg doses) versus placebo control.

FIGS. 8A-8D present graphs showing change from baseline in serum concentrations of HDL-C (FIG. 8A), HDL-CE (FIG. 8B), apoA1 (FIG. 8C) and CE (FIG. 8D) over time, as measured in samples obtained from subjects in cohorts 1-3 following administration of MEDI6012 versus placebo, as described for the multiple ascending dose (MAD) clinical study (MAD Clinical Study D5780000005) in Example 2 herein. Dose-dependent increases in HDL-C, HDL-CE, apoA1 and CE over time were found in the subjects of cohorts 1-3 who received a multiple dosing regimen of MEDI6012 (i.e., 40 mg, 120 mg, or 300 mg of MEDI6012 dosed IV on Days 1, 8, and 15) versus placebo in the MAD study. The dose-dependent increases of the foregoing products (biomarkers) measured in samples from the subjects are consistent with the mechanism of action of LCAT as understood by the skilled practitioner.

FIGS. 9A and 9B present a graph and an area under the concentration curve (AUC) box plot showing LDL-C levels in subjects from cohorts 1-3 following administration of MEDI6012 as described for the MAD study in Example 2 herein. FIG. 9A shows change from baseline in serum concentration of LDL-C (direct measure by a standard laboratory test) over time in samples obtained from subjects in cohorts 1-3 versus placebo (as described in FIGS. 8A-8D above and in Example 2). FIG. 9B shows the AUC_{0-96 h} of LDL-C for subjects of cohorts 1 and 2 in the MAD study of Example 2. An increase in LDL-C was observed after the first 120 mg dose of MEDI6012 and after the third dose of both 40 mg and 120 mg. However, the LDL-C increases were not considered detrimental in view of the static (or decreased) levels of apoB that were concomitantly measured in the subjects. (See, FIGS. 10A and 10B below).

FIGS. 10A and 10B present a graph and an AUC box plot showing apoB levels in subjects from cohorts 1-3 following administration of MEDI6012 as described for the MAD study in Example 2 herein. FIG. 10A shows change from baseline in serum concentration of apoB over time in samples obtained from subjects in cohorts 1-3 versus placebo (as described in FIGS. 8A-8D above and in Example 2). FIG. 10B shows the AUC_{0-96 h} of apoB for subjects of cohorts 1 and 2 in the MAD study of Example 2. No increases in apoB were observed, indicating that there was no detrimental increase in LDL particles associated with the MEDI6012 doses and dosing regimens.

FIGS. 11A and 11B present graphs showing change from baseline in serum concentrations of total cholesterol (TC), (FIG. 11A) and free cholesterol (FC), (FIG. 11B) over time, as measured in samples obtained from subjects in cohorts 1-3 following administration of doses of MEDI6012 (80 mg, 120 mg, or 300 mg) versus placebo, as described for the MAD study in Example 2 herein.

FIGS. 12A and 12B present graphs showing baseline adjusted HDL levels (FIG. 12A) and apoA1 levels (FIG. 12B), in mg/dL, predicted and expected by modeling/simulation analyses in subjects' samples (serum) following dosing of subjects via IV push over 1 minute with a loading dose of MEDI6012 in the indicated amounts of 160 mg, 200 mg, 240 mg, 280 mg and 320 mg, as described in Examples 2 and 3 herein. The modeling/simulation analyses and assessments referred to above and in the figure descriptions infra were conducted based, in large part, on data and results obtained from the single ascending dose (SAD) and the multiple ascending dose (MAD) clinical studies as described in Examples 1 and 2 herein.

FIGS. 13A and 13B present graphs showing baseline adjusted HDL concentration (FIG. 13A) and apoA1 concentration (FIG. 13B), in mg/dL, over time predicted and expected by modeling/simulation analyses of dosing subjects via IV push over 1 minute with MEDI6012 in a dosing regimen of 300 mg (loading dose) on Day 1; 150 mg on Day 3; and 100 mg (maintenance dose) on Day 10.

FIG. 14A presents a graph showing increases in HDL2 as modeling/simulation analysis selection criteria for doses of the LCAT enzyme following intravenous (IV) or subcutaneous (SC) administration of MEDI6012 versus placebo. Shown in the figure are serum levels of HDL2 (mg/dL) over time following IV dosing of MEDI6012 in an amount of 24 mg, 80 mg, 240 mg, or 800 mg, or following SC dosing of MEDI6012 in an amount of 80 mg or 600 mg. HDL2 is a beneficial, cardioprotective subclass of HDL that more readily accepts sphingosine-1-phosphate (S1P), which is a cardioprotective factor. As observed, doses of MEDI6012 in amounts greater than 240 mg do not result in further increases in HDL2. FIG. 14B and FIG. 14C show that HDL2 is the HDL subspecies that carries and accepts more sphingosine-1-phosphate (S 1P) compared to HDL-3, as reported by Sattler, K. et al. (2015, J. Am. Coll. Cardiol., 66:1470-1485).

FIGS. 15A-15D present graphs showing predicted, baseline adjusted HDL-C concentration (mg/dL) over time based on modeling/simulation analysis results using selection criteria for loading and maintenance doses of MEDI6012 that achieve serum HDL-C levels of >60 mg/dL (baseline=35). FIG. 15A shows the predicted and expected results of HDL-C concentration (mg/dL) over time using different loading doses (LD) of MEDI6012 (LD of 160 mg, 200 mg, 240 mg, 280 mg, or 320 mg) and a maintenance dose of MEDI6012 of 160 mg. FIG. 15B shows the predicted and expected results of HDL-C concentration (mg/dL) over time using different loading doses (LD) of MEDI6012 (LD of 160 mg, 200 mg, 240 mg, 280 mg, or 320 mg) and a maintenance dose of MEDI6012 of 100 mg. FIG. 15C shows the predicted and expected results of HDL-C concentration (mg/dL) over time using different loading doses (LD) of MEDI6012 (LD of 160 mg, 200 mg, 240 mg, 280 mg, or 320 mg) and a maintenance dose of MEDI6012 of 120 mg. FIG. 15D shows the predicted and expected results of HDL-C concentration (mg/dL) over time using different loading doses (LD) of MEDI6012 (LD of 160 mg, 200 mg, 240 mg, 280 mg, or 320 mg) and a maintenance dose of MEDI6012 of 80 mg.

FIGS. 16A-16D present graphs showing predicted, baseline adjusted apoA1 concentration (mg/dL) over time based on modeling/simulation analysis results using selection criteria for loading and maintenance doses of MEDI6012 that maintain steady state apoA1 levels. FIG. 16A shows the predicted and expected results of apoA1 concentration (mg/dL) over time using different loading doses (LD) of MEDI6012 (LD of 160 mg, 200 mg, 240 mg, 280 mg, or 320 mg) and a maintenance dose of MEDI6012 of 160 mg. FIG. 16B shows the predicted and expected results of apoA1 concentration (mg/dL) over time using different loading doses (LD) of MEDI6012 (LD of 160 mg, 200 mg, 240 mg, 280 mg, or 320 mg) and a 100 mg maintenance dose of MEDI6012. FIG. 16C shows the predicted and expected results of apoA1 concentration (mg/dL) over time using different loading doses (LD) of MEDI6012 (LD of 160 mg, 200 mg, 240 mg, 280 mg, or 320 mg) and a 120 mg maintenance dose of MEDI6012. FIG. 16D shows the predicted and expected results of apoA1 concentration (mg/dL) over time using different loading doses (LD) of MEDI6012 (LD of 160 mg, 200 mg, 240 mg, 280 mg, or 320 mg) and an 80 mg maintenance dose of MEDI6012.

FIG. 17 presents a graph showing total small LDL particles (LDL-P), (nmol/L), over time as observed for different doses of MEDI6012 (administered IV or SC) that achieve a decrease the amount of small LDL-P. The MEDI6012 dose groups included IV dosing in an amount of 24 mg, 80 mg, 240 mg and 800 mg; and SC dosing in an amount of 80 mg SC versus placebo. The decrease in small LDL-P was determined to be about 40-41% at a dose of MEDI6012 in an amount of 80 mg (IV) and 80% at a dose of MEDI6012 in an amount of 240 mg (IV).

FIGS. 18A-18D present graphs showing predicted, baseline adjusted cholesteryl ester (CE) (mg/dL) over time based on modeling/simulation analysis results using selection criteria for loading and maintenance doses of MEDI6012 that result in minimal or no CE accumulation. FIG. 18A shows the predicted and expected results of CE concentration (mg/dL) over time using different loading doses (LD) of MEDI6012 (LD of 160 mg, 200 mg, 240 mg, 280 mg, or 320 mg) and a 160 mg maintenance dose of MEDI6012. FIG. 18B shows the predicted and expected results of CE concentration (mg/dL) over time using different loading doses (LD) of MEDI6012 (LD of 160 mg, 200 mg, 240 mg, 280 mg, or 320 mg) and a 100 mg maintenance dose of MEDI6012. FIG. 18C shows the predicted and expected results of CE concentration (mg/dL) over time using different loading doses (LD) of MEDI6012 (LD of 160 mg, 200 mg, 240 mg, 280 mg, or 320 mg) and a 120 mg maintenance dose of MEDI6012. FIG. 18D shows the predicted and expected results of CE concentration (mg/dL) over time using different loading doses (LD) of MEDI6012 (LD of 160 mg, 200 mg, 240 mg, 280 mg, or 320 mg) and an 80 mg maintenance dose of MEDI6012.

FIGS. 19A-19D present graphs showing predicted, baseline adjusted HDL-CE (mg/dL) over time based on modeling/simulation analysis results using selection criteria for loading and maintenance doses of MEDI6012 that achieve suitable HDL-CE levels in serum following dosing. FIG. 19A shows the predicted and expected results of HDL-CE concentration (mg/dL) over time using different loading doses (LD) of MEDI6012 (LD of 160 mg, 200 mg, 240 mg, 280 mg, or 320 mg) and a 160 mg maintenance dose of MEDI6012. FIG. 19B shows the predicted and expected results of HDL-CE concentration (mg/dL) over time using different loading doses (LD) of MEDI6012 (LD of 160 mg, 200 mg, 240 mg, 280 mg, or 320 mg) and a 100 mg maintenance dose of MEDI6012. FIG. 19C shows the predicted and expected results of HDL-CE concentration (mg/dL) over time using different loading doses (LD) of MEDI6012 (LD of 160 mg, 200 mg, 240 mg, 280 mg, or 320 mg) and a 120 mg maintenance dose of MEDI6012. FIG. 19D shows the predicted and expected results of HDL-CE concentration (mg/dL) over time using different loading doses (LD) of MEDI6012 (LD of 160 mg, 200 mg, 240 mg, 280 mg, or 320 mg) and an 80 mg maintenance dose of MEDI6012. Both FIGS. 18A-18D and 19A-19D show that while CE accumulation occurs, it occurs in LDL and not in HDL-CE. In accordance with the described methods, maintenance doses of LCAT (e.g., rhLCAT or MEDI6012) administered to subjects are those that result in minimal or no CE accumulation.

FIGS. 20A-D present graphs showing observed doses of MEDI6012 that achieved few or no VVL-HDL particles and few VL-HDL particles (mg/dL) resulting from activity of the LCAT enzyme (MEDI6012) following administration to subjects in the SAD study. As observed from the analysis results, a 240 mg dose of MEDI6012 resulted in a 2 mg/dL increase in VVL-HDL and a 17 mg/dL increase in VL-HDL. A dose of 80 mg of MEDI6012 resulted in no increase in VVL-HDL and a 2 mg/dL increase in VL-HDL.

FIGS. 21A-21C present schematic depictions of the modeling parameters employed for the modeling/predictions performed for MEDI6012 IV dosing for cohort 4 in the MAD study, as based on modeling performed for rhLCAT ACP501 dosing associated with reverse cholesterol transport (RCT), as reported by Bosch, R. et al., Poster entitled “A mechanism-based model is able to simultaneously explain the effect of rhLCAT and HDL mimetics on biomarkers of reverse cholesterol transport,” presented at the 2015 Population Approach Group in Europe (PAGE) Meeting, Hersonissos, Crete, Greece; (Example 3 infra). FIG. 21A shows a schematic overview of model characteristics. FIG. 21B shows a schematic representation of the integrated model for RCT. In FIGS. 21A and 21B, 1) indicates small preβ-HDL particles in the blood stream acquire cholesterol from the peripheral tissue. 2) shows that LCAT catalyzes the conversion of cholesterol to CE; 3) shows that CE moves to the center of the HDL particle hereby turning it into a large α-HDL; and 4) shows that CE in αHDL is returned to the liver either 4a) directly or 4b) via LDL by CETP and LDL receptors on the liver. In the schematic depictions, bold parameters: Fixed to literature values; black parameters: Derived based on steady state conditions (k84 fixed to 10 h⁻¹); and grey parameters: Re-estimated in Nonmem after inclusion of CSL112 data. FIG. 21C presents a description of the integration of HDL-C and apoA1 into one model as illustrated in FIGS. 21A and 21B.

FIGS. 22A and 22B show graphs of serum concentrations of HDL-C over time in subjects who received a 300 mg dose of MEDI6012 (Day 1), followed by a 150 mg dose of MEDI6012 (Day 3), followed by a 100 mg dose of MEDI6012 (Day 10) by IV push versus placebo, as determined in the MAD study described in Example 3 herein. FIG. 22A shows the serum concentration of HDL-C over time in subjects administered the IV push dosage regimen of MEDI6012 versus placebo control. FIG. 22B shows the change from baseline in serum concentration of HDL-C over time in subjects administered the IV push dosage regimen of MEDI6012 versus placebo control.

FIGS. 23A and 23B show graphs of the serum concentration (levels) of LDL-C (direct measure by a standard laboratory test) over time in subjects who received a 300 mg dose of MEDI6012 (Day 1), followed by a 150 mg dose of MEDI6012 (Day 3), followed by a 100 mg dose of MEDI6012 (Day 10) by IV push compared with placebo as determined in the MAD study described in Example 3 herein. FIG. 23A shows the serum concentration of LDL-C (direct measure by a standard laboratory test) over time in subjects administered the IV push dosage regimen of MEDI6012. FIG. 23B shows the change from baseline in serum concentration of LDL-C (direct measure by a standard laboratory test) over time in subjects administered the IV push dosage regimen of MEDI6012.

FIGS. 24A and 24B show graphs of the concentration of apolipoprotein B (apoB) in the serum of subjects who received a 300 mg dose of MEDI6012 (Day 1), followed by a 150 mg dose of MEDI6012 (Day 3), followed by a 100 mg dose of MEDI6012 (Day 10) by IV push compared with placebo as determined in the MAD study described in Example 3 herein. FIG. 24A shows the serum concentration of apoB over time in subjects administered the IV push dosage regimen of MEDI6012. FIG. 24B shows the change from baseline in serum concentration of apoB over time in subjects administered the IV push dosage regimen of MEDI6012.

FIGS. 25A and 25B show graphs of serum concentrations of apoA1 over time in subjects who received a 300 mg dose of MEDI6012 (Day 1), followed by a 150 mg dose of MEDI6012 (Day 3), followed by a 100 mg dose of MEDI6012 (Day 10) by IV push versus placebo, as determined in the MAD study described in Example 3 herein. FIG. 25A shows the serum concentration of apoA1 over time in subjects administered the IV push dosage regimen of MEDI6012. FIG. 25B shows the change from baseline in serum concentration of apoA1 over time in subjects administered the IV push dosage regimen of MEDI6012.

FIG. 26A shows the baseline adjusted levels of HDL-C obtained from modelling/simulation analyses (the solid and dashed lines) compared to the observed data (the individual data points: circles and squares) from administration of MEDI6012 in Cohort 3 and Cohort 4 of the MAD study (Day 0 to Day 70). The modeling/simulation analyses referred to above and in the figure descriptions infra were conducted based, in large part, on data and results obtained from the SAD and the MAD clinical studies as described in Examples 1 and 2 herein. FIG. 26B shows the predicted model (dashed line) and the observed data (circles) from administration of MEDI6012 in Cohort 4 of the MAD study alone (Day 0 to Day 70). FIG. 26C shows the baseline adjusted levels of HDL-C obtained from modelling/simulation analyses (the solid and dashed lines) compared to the observed data (the individual data points: circles and squares) from administration of MEDI6012 in Cohort 3 and Cohort 4 of the MAD study (Day 0 to Day 5).

FIGS. 27A-D show the observed results from all cohorts (Cohorts 1-4) of the MAD study, as defined in Examples 2 and 3 herein. Subjects in Cohort 1 of the MAD study were administered via IV infusion a dose of 40 mg of MEDI6012 on Days 1, 8 and 15. Subjects in Cohort 2 of the MAD study were administered via IV infusion a dose of 120 mg of MEDI6012 on Days 1, 8 and 15. Subjects in Cohort 3 of the MAD study were administered via IV infusion a dose of 300 mg of MEDI6012 on Days 1, 8 and 15. Subjects in Cohort 4 of the MAD study were administered via IV push a dose of 300 mg of MEDI6012 on Day 1, followed by a dose of 150 mg of MEDI6012 on Day 3, followed by a dose of 100 mg of MEDI6012 on Day 10. FIG. 27A shows the observed change from baseline in serum concentration of HDL-C over time from Cohorts 1-4 of the MAD study. FIG. 27B shows the observed change from baseline in serum concentration of ApoA1 over time from Cohorts 1-4 of the MAD study. FIG. 27C shows the observed change from baseline in serum concentration of LDL-C (Direct) over time from Cohorts 1-4 of the MAD study. FIG. 27D shows the observed change from baseline in serum concentration of ApoB over time from Cohorts 1-4 of the MAD study.

FIGS. 28A-28D present area under the concentration curve (AUC) box plots from 0 to 96 hours after the 1^{st} dose and after the 3^{rd} dose showing HDL-C, ApoA1, LDL-C and ApoB levels in subjects from Cohorts 1-4 following administration of MEDI6012 as described for the MAD study in Examples 2 and 3 herein. FIG. 28A shows the AUC_{0-96 h} of HDL-C for subjects of Cohorts 1-4 of the MAD study. FIG. 28B shows the AUC_{0-96 h} of ApoA1 for subjects of Cohorts 1-4 of the MAD study. FIG. 28C shows the AUC_{0-96 h} of LDL-C for subjects of Cohorts 1-4 of the MAD study. FIG. 28D shows the AUC_{0-96 h} of ApoB for subjects of Cohorts 1-4 of the MAD study.

### DETAILED DESCRIPTION OF THE INVENTION

The present disclosure features methods of treating and affording protection against heart disease, coronary heart disease and/or other cardiac-associated diseases and conditions by administering to subjects (patients) in need, a purified and isolated human lecithin cholesterol acyltransferase (LCAT) enzyme, in particular, a recombinant human lecithin cholesterol acyltransferase (rhLCAT) enzyme, called MEDI6012 herein, (previously known as ACP501) at newly developed, clinically beneficial doses and dosing regimens as described herein.

The present methods provide therapeutically and/or prophylactically effective doses of the LCAT enzyme via administration of rhLCAT or MEDI6012 to subjects (patients) for the treatment of a number of heart-related diseases and conditions. The methods provide directly to subjects the LCAT enzyme, which plays an active role in esterifying free cholesterol to cholesteryl ester (CE), to facilitate the maturation of high-density lipoprotein (HDL) particles and to increase and maintain therapeutic plasma and serum concentrations of products of lipid metabolism, e.g., apoA1 and/or functional HDL-C, which are associated with a lower risk of heart disease and atherosclerosis. Thus, the presently-described methods involving doses and dosing regimens of the LCAT enzyme, such as rhLCAT or MEDI6012, afford effective treatment and protection against heart- and heart-related diseases and pathologies, such as stroke (ischemic stroke), atherosclerosis, myocardial infarction, or myocardiocyte apoptosis, to subjects (patients) in need, for example, patients experiencing acute or chronic cardiac events that threaten their immediate and long-term cardiac function and their overall health.

In particular, the present methods provide effective therapeutic benefit related to the use of the described doses of rhLCAT or MEDI6012 and treatment regimens involving rhLCAT or MEDI6012 dosing schedules for treating a subject having heart disease, coronary heart disease and/or other heart-associated diseases or conditions, for example, acute or chronic heart disease, cardiovascular disease, coronary artery disease (CAD), stable CAD, atherosclerosis, atherosclerotic cardiovascular disease (CVD), stable CVD, unstable CVD, acute coronary syndrome (ACS), heart failure (HF), congestive HF, hospitalized HF, heart failure with reduced ejection fraction (EF), heart failure with preserved EF, ST-elevated myocardial infarction (STEMI), non-STEMI, or a disease, pathology, or condition related to or associated with heart or cardiac disease, familial or acquired, such as stroke, ischemic stroke, myocardial disease, peripheral artery disease, myocardial infarction, ischemic cardiomyopathy, non-ischemic cardiomyopathy, chemotherapy-induced cardiomyopathy, cerebrovascular disease, acute or chronic renal disease, and/or symptoms thereof, without limitation as to cause.

The practice of the present methods results in an increase in LCAT enzyme, and thus the activity of the enzyme, in a subject treated with rhLCAT or MEDI6012, which, in turn, produces cholesteryl ester (CE) so as to increase CE levels in the subject. Accordingly, the increase in LCAT activity level and/or the production of cholesteryl ester may serve as markers of efficacy of therapeutic administration and treatment. The methods described herein further encompass the administration of rhLCAT or MEDI6012 as activating LCAT or playing a role as LCAT activator to increase LCAT activity to therapeutic levels in a subject in need, such as a patient with cardiac disease or coronary artery disease. In some embodiments, the administration of rhLCAT or MEDI6012 at the dosages and according to the dosage regimens described herein can include another type of LCAT activator, such as a small molecule or a biologic (e.g., peptide, polypeptide or monoclonal antibody).

The practice of the present methods also results in increases in serum and plasma concentrations (levels) of biomarkers such as apoA1 and/or HDL-C that are associated with a reduction in risk of cardiac or cardiovascular disease, e.g., CAD or MI, and with amelioration of the harmful effects caused by cardiac or cardiovascular disease in the body. The methods also result in no change or alteration in (or even a decrease in) the concentrations (levels) of biomarkers such as LDL-C particles and apoB that are associated with increased risk of heart disease or detrimental outcome of heart disease or treatment. It is to be understood that the terms “dosing or dose regimens,” “treatment regimens,” dosing schedules,” and “treatment schedules” are used interchangeably herein. The terms “subject” and “patient” are also used interchangeably herein.

### Lecithin-Cholesterol Acyltransferase (LCAT) Enzyme

Lecithin-cholesterol acyltransferase (LCAT), a plasma enzyme glycoprotein that is produced and secreted by the liver, catalyzes the production of cholesteryl ester (CE) from free (unesterified) cholesterol and phosphatidylcholine (lecithin) present in plasma lipoproteins. In humans, about 90% of CE in plasma is formed by the LCAT enzyme, and the reaction mostly occurs on HDL (α-LCAT activity) and to a lesser extent on apolipoprotein B (apoB)-containing particles (β-LCAT activity). The esterification of cholesterol by LCAT helps to maintain HDL (HDL-CE) levels by promoting the maturation of small discoidal forms of HDL (called preβ-HDL and α4-HDL particles) into larger spherical forms of HDL (called α1-3-HDL particles), which have a longer half-life. In humans, most HDL-cholesteryl esters (HDL-CEs) are eventually transferred, in exchange for triglycerides (TG), to very low-density lipoproteins (VLDL), intermediate-density lipoproteins and low-density lipoproteins (LDL) by cholesteryl ester transfer protein (CETP).

The amount or concentration of LCAT or LCAT activity in the serum can be determined using several methods known to those having skill in the art, e.g., fluorometric assay. The mass of LCAT can be determined, for example, by a competitive double antibody radioimmunoassay. Routine methods also are known for measuring absolute LCAT activity in a serum or blood sample and for measuring the rate of cholesterol esterification rate. See, e.g., J. J. Albers et al., 1986, Methods in Enzymol., 129:763-783 and M. P. T. Gillett and J. S. Owens, Chapter 7b, Eds.: C. A. Converse and E. R. Skinner, in Lipoprotein Analysis—A Practical Approach, pp. 187-201. By way of nonlimiting example, LCAT activity can be determined by measuring the conversion of radiolabeled cholesterol to cholesteryl ester after incubation of the enzyme and radiolabeled lecithin-cholesterol liposome substrates containing apolipoprotein A1 (apoA1). Endogenous cholesterol esterification rate can be determined by measuring the rate of conversion of labeled cholesterol to cholesteryl ester after incubation of fresh plasma that is labeled with a trace amount of radioactive cholesterol by equilibration with a [¹⁴C]cholesterol-albumin mixture at 4° C. (See, U.S. Pat. No. 6,635,614). The endogenous cholesterol esterification rate is a better measure of the therapeutic LCAT activity, because it reflects not only the amount of LCAT activity present in the serum, but also the nature and amount of substrate and co-factors that are present in plasma. Thus, the cholesterol esterification rate is not necessarily proportional to either the mass of LCAT or the absolute LCAT activity in vivo. In another method, the conversion of free cholesterol to esterified cholesterol by LCAT can be measured using dual-labeled phosphatidylcholine (lecithin) as an LCAT substrate. When uncleaved, the fluorophores in the dual-labeled substrate are in a quenched state, and upon hydrolysis by LCAT at the sn-2 position of phosphatidylcholine, fluorescent monomer chains are produced which can be quantified in a fluorescence microplate reader. (Cell Biolabs, Inc., San Diego, Calif.).

### MEDI6012—Recombinant Human LCAT

MEDI6012 (formerly called ACP501) is recombinant human (rh) lecithin-cholesterol acyltransferase (LCAT), (rhLCAT), an approximately 60 kilodalton, glycosylated, single-chain enzymatic protein consisting of 416 amino acids produced by, and isolated and purified from, Chinese hamster ovary (CHO) cells in cell culture. MEDI6012 and ACP501 have identical amino acid sequences and are therefore considered the same molecular entity. The MEDI6012 product as obtained from CHO cell culture has high levels of enzymatic activity on a per-mg of protein basis and product- and process-related purity that is advantageous for human use.

The present disclosure encompasses methods in which rhLCAT, MEDI6012, is provided in therapeutic doses that are administered to subjects in dosing regimens to treat, reduce, or ameliorate the serious and adverse effects of acute or chronic heart (cardiac) disease, cardiovascular disease, coronary artery disease, atherosclerotic cardiovascular disease (CVD), acute coronary syndrome (ACS) and/or symptoms thereof. In an embodiment, methods involving therapeutic administration of rhLCAT or MEDI6012 are provided to reduce the risk of ischemic events as adjunct to the standard of care in patients with ACS. In another embodiment, the administration of effective dosage amounts of rhLCAT or MEDI6012 affords cardiotherapeutic, cardioprotective, and anti-atherogenic (atheroprotective) effects and myocardioprotective effects by preventing myocardial fibrosis and hypertrophy in a subject. In other embodiments, the administration of effective dosage amounts of rhLCAT or MEDI6012 treats and/or provides protective effects against acute or chronic heart disease, cardiovascular disease, coronary artery disease (CAD), stable CAD, atherosclerosis, atherosclerotic cardiovascular disease (CVD), stable CVD, unstable CVD, acute coronary syndrome (ACS), heart failure (HF), congestive HF, hospitalized HF, heart failure with reduced ejection fraction (EF), heart failure with preserved EF, ST-elevated myocardial infarction (STEMI), non-STEMI, or a disease, pathology, or condition related to or associated with heart or cardiac disease, familial or acquired, such as stroke, ischemic stroke, myocardial disease, peripheral artery disease, myocardial infarction, ischemic cardiomyopathy, non-ischemic cardiomyopathy, chemotherapy-induced cardiomyopathy, acute or chronic renal disease, and/or symptoms thereof.

Without wishing to be bound by a particular theory, the administration of MEDI6012 to patients with heart disease and/or acute coronary disease serves to upregulate mobilization of cholesterol from tissues, including cholesterol from atherosclerotic plaques in coronary arteries, resulting in their stabilization and a consequent decreased risk for recurrent major adverse cardiovascular events. MEDI6012 beneficially provides enhanced HDL maturation, HDL function and reverse cholesterol transport (RCT) from tissues to the liver for removal. In addition, as described and exemplified herein, the administration of MEDI6012 (“MEDI6012 dosing”) to patients is well tolerated and does not cause clinical pathologies or adverse changes in the body condition of patients to whom it is delivered.

Atherosclerosis, the underlying condition of atherosclerotic cardiovascular disease (CVD), is a progressive condition associated with significant comorbidity and mortality in afflicted patients. Excess cholesterol in arteries induces numerous detrimental effects, such as inflammation, a decrease in endothelium-dependent vasorelaxation, and promotion of plaque instability. Periods of plaque instability can result in acute coronary syndrome (ACS), a spectrum of life-threatening clinical conditions that include unstable angina and heart attack, i.e., non-ST- and ST-segment elevation myocardial infarction (non-STEMI (NSTEMI) and STEMI, respectively).

Plaque rupture is caused by the dissolution of the fibrous cap; the dissolution itself results from the release of metalloproteinases (collagenases) from activated inflammatory cells, which is followed by platelet activation and aggregation, activation of the coagulation pathway, and vasoconstriction. Typical or standard treatment for ACS is focused on drugs that rapidly inhibit platelet aggregation and/or blood clot formation, e.g., antiplatelet agents including aspirin and the adenosine diphosphate receptor antagonists, such as clopidogrel, prasugrel, and ticagrelor, which can be given orally, together with the IV-administered IIb/IIIa receptor antagonists abciximab, eptifibatide, and tirofiban.

Commonly used anticoagulants include low-molecular weight heparins, thrombin inhibitors, and Factor Xa inhibitors. To date, drug therapies, as well as percutaneous coronary interventions (PCI); balloon angioplasty and stent deployment, have been focused only on the culprit lesion and do not adequately address the underlying cause of plaque vulnerability for rupture (i.e., cholesterol deposition) or reduce the risk of new plaque ruptures at other sites. While chronic lipid lowering therapy with statins reduces the risk of both primary and secondary cardiovascular (CV) events by lowering plasma low-density lipoprotein-cholesterol (LDL-C), statins do not acutely stabilize artery-clogging plaque.

The methods described herein involving the administration of rhLCAT or MEDI6012 at therapeutic (and cardio- and myocardio-protective) doses and dose regimens afford to patients advantageous and beneficial therapies with a number of positive outcomes for patients' cardiac and cardiovascular disease treatment and improvement of cardiovascular conditions and symptoms thereof, namely, rapid removal of plaque cholesterol, stabilization of vulnerable plaques in ACS patients, prevention of apoptosis in myocardiocytes and reduction of the likelihood of subsequent ischemic events, which can be effective in both the carotid and peripheral vasculature.

The methods involving the administration of doses and dosing regimens of rhLCAT or MEDI6012 to subjects as described herein provide increases in HDL (HDL-C) and/or apoA1 that are cardioprotective in acute myocardial infarction (MI). Because LCAT administration rapidly increases the levels of both HDL and/or apoA1, the treatment methods are especially advantageous for acute treatment. The advantages and benefits of the methods described herein comport well with reports of epidemiologic and preclinical studies, which have established that higher levels of HDL-C are cardioprotective in patients post-MI and that infusions of HDL or apoA1 mimetics reduce myocardial infarct size and improve left ventricular systolic function in animal models of acute MI. By way of example, post infarct ejection fraction (EF) is lower in patients with low HDL-C, even after excluding baseline coronary heart disease (CHD), (Wang T D, et al., 1998, Am J Cardiol, 81:531-537; Kempen H J, et al., 1987, J Lab Clin Med, 109:19-26); infusion of the apoA1 mimetic CSL-111 in two different mouse models of acute MI demonstrated an increase in viable myocardium of 54%-61%, a reduction in infarct size by 21%-26%, and reduction in the recruitment of leukocytes and neutrophils in the area of infarction (Heywood, S. E. et al, 2017, Sci. Transl. Med., 9(411), DOI: 10.1126/scitranslmed.aam6084); infusion of the apoA1 mimetic ETC-216 in a rabbit model of ischemia-reperfusion resulted in a marked reduction in infarct size (Marchesi et al., 2004, J Pharmacol Exp Ther., 311(3):1023-31); adenoviral transfer of apoA1 2 weeks prior to MI in a mouse model achieved apoA1 levels that were 1.5 times greater than controls and showed increased survival (˜2×), attenuated infarct expansion, inhibition of left ventricle (LV) dilation, and improved hemodynamics (Gordts et al, 2013, Gene Therapy, 20, 1053-1061); infusion of HDL versus HDL and its constituent sphingosine-1-phosphate (S1P) in a mouse model of ischemia-reperfusion showed a 20% reduction in infarct size with HDL alone and a 40% reduction in infarct size when HDL and S1P (Theilmeier et al, 2006, Circulation, 114:1403-1409); and ApoA1 infusions reduced infarct size in Wistar rats via the RISK/SAFE pro-survival kinase pathways (Akt, ERK1/2, STAT-3) (Kalakech et al, 2014, PLoS ONE, 9(9): e107950). The increases in apoA1 reported by Gordt et al, and Marchesi et al, are similar to the increases in apoA1 found following the administered of MEDI6012 (rhLCAT) at the doses described herein. Accordingly, the methods as described herein provide doses of MEDI6012 that are especially beneficial for acute treatment and for the likelihood of reducing myocardial infarct size by increasing levels of HDL-C and/or apoA1. Smaller infarct size is predictive of better clinical outcomes, namely, less heart failure and better survival (Stone, G. W. et al., 2016, J Am Coll Cardiol, 67(14):1674-83).

Treatment Methods Involving rhLCAT (MEDI6012) Administration

The methods described herein afford medical and clinical benefits associated with the administration of doses and dosing schedules (also called dosing regimens or treatment regimens herein) of rhLCAT or MEDI6012 (or a pharmaceutically acceptable composition or formulation thereof) to a subject who is in need of treatment, for example, a subject who has, without limitation, heart disease, coronary heart disease, or coronary artery disease (atherosclerosis). In some embodiments, the treatment methods described herein were developed based on clinical study results in human subjects. In some embodiments, the treatment methods described herein were developed from ex vivo modeling and simulation analyses that were based on preclinical study results, as well as clinical study data and results in human subjects. In all cases, the methods led to the discovery and surprisingly beneficial effectiveness of doses of rhLCAT or MEDI6012, and dosing regimens involving rhLCAT or MEDI6012, for administration to subjects to achieve favorable and advantageous therapeutic and protective results in the treated individuals, with limited and/or manageable unwanted side effects or off-target effects.

The beneficial therapeutic effects following the administration of doses and dosing regimens of rhLCAT or MEDI6012 to subjects as described herein were assessed by measuring and evaluating the concentrations (levels) of several different components of cholesterol and lipid metabolism in biological samples, e.g., blood, plasma, or serum, obtained from the treated subjects during and following the treatment (dosing) regimens.

Single Dose Treatment Methods Involving rhLCAT (MEDI6012)

In general, single ascending dose (SAD) studies involve a small group of subjects who receive a single dose of a compound or drug in a clinical setting, usually in a clinical research unit or CRU. During the studies, subjects are monitored closely for safety, and pharmacokinetic (PK) assessments are performed for a predetermined time. If the compound is deemed to be well tolerated, and the PK data are generally as is expected, dose escalation occurs, either within the same group or in another group of healthy subjects, according to the approved protocol. Dose escalation usually continues until the maximum dose has been attained according to the protocol unless predefined maximum exposure is reached, or intolerable side effects become apparent. In addition, dose escalation may be discontinued (or may proceed more cautiously than planned) if there is evidence of a supra-proportional relationship between dose and exposure, such that exposures at higher dose levels become difficult to predict. SAD studies usually include sequential groups in a parallel design for maximum exposure, or may be of a crossover design to provide more information on dose linearity. To minimize bias effects, subjects are usually randomly assigned to treatment using computer generated, statistical randomization codes. Such studies are also usually placebo controlled to determine whether the observed effects are due to the study drug or to environmental conditions, and are often conducted in a single (subject) blinded manner to allow informed decision on dose escalation, with safety and PK data being available for investigator review.

In one aspect, the present disclosure provides treatment methods as described herein involving the administration of one or more doses of the active drug, namely, an isolated and purified LCAT enzyme, e.g., rhLCAT or MEDI6012, to treat a subject who has acute or chronic heart disease, cardiovascular disease, coronary artery disease (CAD), stable CAD, atherosclerosis, atherosclerotic cardiovascular disease (CVD), stable CVD, acute coronary syndrome (ACS), or a disease or condition related to or associated with heart or cardiac disease, such as stroke, ischemic stroke, myocardial disease, myocardial infarction, and the like, and/or symptoms thereof. In a particular embodiment, a single dose of the isolated and purified LCAT enzyme, e.g., rhLCAT or MEDI6012, is administered in the method. In another particular embodiment, the subject has stable coronary artery disease (CAD). Such dosing methods were developed, in part, based on SAD clinical studies in which various doses of MEDI6012 were administered to subjects whose responses and levels of cholesterol and lipid metabolism components, products and by-products (e.g., pharmacodynamic (PD) markers) were assessed following the administration of MEDI6012. (See, Example 1). Such PD markers, which may be evaluated in a sample obtained from a subject prior to, during and/or following administration of MEDI6012 to the subject. The evaluated PD markers include, without limitation, HDL-C, as well as additional lipids and lipoproteins whose levels are assessed and/or measured to describe and quantify the effects of MEDI6012 on the cholesterol and lipid pathways, including, but not limited to, total cholesterol (TC), free cholesterol (FC), which is non-esterified, cholesteryl ester (CE), HDL-esterified cholesterol (HDL-CE), HDL-unesterified cholesterol (HDL-UC), non-HDL-C, non-HDL-CE, non-HDL-UC, LDL-C (direct measure by standard laboratory test), VLDL-C, TG, apoB, apoA1, apoA1l, apoCIII, or apoE. By way of example, immunoassays, such as an enzyme-linked immunosorbent assay (ELISA), may be used to characterize and quantify preβ1-HDL. Lipoprotein size and particle number for HDL, LDL and VLDL can be characterized by nuclear magnetic resonance (NMR), (LipoScience, Inc., Raleigh, N.C.). In an embodiment, the sample obtained from the subject is a blood, serum, or plasma sample.

In an embodiment, the methods involve administering to a subject in need MEDI6012 in an amount of from 20-2000 mg. In an embodiment, a dose of 24-1600 mg of MEDI6012 is administered to the subject. In an embodiment, a dose of 24-800 mg of MEDI6012 is administered to the subject. In embodiments, a dose of 20, 24, 30, 35, 40, 45, 50, 55, 60, 65, 70, 75, 80, 85, 90, 95, 100, 120, 140, 150, 160, 180, 200, 220, 240, 260, 280, 300, 320, 340, 360, 380 mg, 400, 420, 440, 460, 480, 500, 520, 540, 560, 580, 600, 620, 640, 660, 680, 700, 720, 740, 760, 780, 800, 8220, 840, 860, 880, 900, 920, 940, 960, 980, 1000, 1020, 1040, 1060, 1080, 1100, 1200, 1300, 1400, 1600, 1700, 1800, 1900, or 2000 milligrams (mg), including values therebetween, of MEDI6012 is administered to a subject in need. As will be appreciated by the skilled practitioner in the art, for a typical patient, e.g., a patient with coronary artery disease (CAD), weighing about 80 kg, a dose of 24 mg is equivalent to approximately 0.3 mg/kg; a dose of 80 mg is equivalent to approximately 1 mg/kg; a dose of 240 mg is equivalent to approximately 3 mg/kg; a dose of 800 mg is equivalent to approximately 10 mg/kg; and a dose of 1600 mg is equivalent to approximately 20 mg/kg. In embodiments, the subject in need is afflicted with acute or chronic heart disease, cardiovascular disease, coronary artery disease (CAD), stable CAD, atherosclerosis, atherosclerotic cardiovascular disease (CVD), stable CVD, unstable CVD, acute coronary syndrome (ACS), heart failure (HF), congestive HF, hospitalized HF, heart failure with reduced ejection fraction (EF), heart failure with preserved EF, ST-elevated myocardial infarction (STEMI), non-STEMI, or a disease, pathology, or condition related to or associated with heart or cardiac disease, familial or acquired, such as stroke, ischemic stroke, myocardial disease, peripheral artery disease, myocardial infarction, ischemic cardiomyopathy, non-ischemic cardiomyopathy, chemotherapy-induced cardiomyopathy, cerebrovascular disease, acute or chronic renal disease, and/or symptoms thereof. In a particular embodiment, the subject has stable coronary artery disease (CAD).

In an embodiment, the methods involve parenterally administering a dose of MEDI6012 to a subject in need thereof. In an embodiment, the methods involve intravenously administering a dose of MEDI6012 to a subject in need. In an embodiment, the dose of MEDI6012 is administered to the subject by intravenous (IV) infusion. In an embodiment, the methods involve subcutaneously administering a dose of MEDI6012 to a subject in need. In an embodiment, the dose of MEDI6012 is administered intravenously to the subject over a time period of from minutes to hours. In an embodiment, the methods involve administering a dose of MEDI6012 to a subject by IV or SC delivery over a time period of from about or equal to 30 seconds, 1 minute, 3 minutes, 5 minutes, 10 minutes, 15 minutes, 30 minutes, 1 hour, 2 hours, 3 hours, 4 hours, or 5 hours, including times therebetween, particularly after a subject with a heart condition, cardiovascular disease, or atherosclerotic condition presents at a medical facility (e.g., a hospital, clinic, urgent care center, medical practitioner's office), or at a site where a medical professional or clinician is in attendance or is able to assist in administering the dose of MEDI6012 to the subject.

In an embodiment, the methods involve administering a dose of MEDI6012 to a subject immediately or within a short time period, such as minutes, for example, over a time period of about or equal to 30 seconds to 10 minutes, or over a time period of about or equal to 1-5 minutes, or over a time period of about or equal to within 1-3 minutes, or over a time period of about or equal to 1-2 minutes, and times therebetween, upon presentation of a subject with a heart condition or atherosclerotic condition at a medical facility or site. In an embodiment, MEDI6012 is administered intravenously to a subject by IV push over a short time period, such as those noted supra. In an embodiment, a bolus dose or loading dose of MEDI6012 is administered intravenously to a subject by IV push. In other embodiments, the methods involve administering a dose of MEDI6012 to a subject by IV infusion or by SC administration (e.g., SC injection), over a longer period of time following presentation of the subject at a medical facility or site, or during the subject's stay at the medical facility or site. In an embodiment, the dose of MEDI6012 is administered to the subject by IV infusion over a time period of about or equal to 30 minutes to 3 hours, or over a time period of about or equal to 1 minute to 3 hours. In an embodiment, the dose of MEDI6012 is administered to the subject by IV infusion over a time period of about or equal to 30 minutes to 1 hour. In a particular embodiment, the dose of MEDI6012 is administered to the subject by IV infusion over a time period of about or equal to 1 hour. In embodiments, a dose of 24, 80, 240, 600, 800 mg, or 1600 mg of MEDI6012 is administered to the subject intravenously. In an embodiment, a dose of 24 mg of MEDI6012 is intravenously administered to the subject. In an embodiment, a dose of 80 mg is intravenously administered to the subject. In an embodiment, a dose of 240 mg of MEDI6012 is intravenously administered to the subject. In an embodiment, a dose of 600 mg of MEDI6012 is intravenously administered to the subject. In an embodiment, a dose of 800 mg of MEDI6012 is intravenously administered to the subject. In any of the foregoing embodiments, one or more of the above-stated doses of MEDI6012 is intravenously administered to the subject.

In an embodiment, a dose of MEDI6012 is subcutaneously administered to the subject, e.g., by subcutaneous (SC) infusion or injection. In an embodiment, a dose of 80 or 600 mg of MEDI6012 is administered to the subject subcutaneously. In a particular embodiment, a dose of 80 mg of MEDI6012 is administered to the subject subcutaneously. In a particular embodiment, a dose of 600 mg of MEDI6012 is administered to the subject subcutaneously. In any of the foregoing embodiments, one or more of the above-stated doses of MEDI6012 is subcutaneously administered to the subject.

In embodiments of any aspect of the methods described herein, the subject has acute or chronic heart disease, cardiovascular disease, coronary artery disease (CAD), stable CAD, atherosclerosis, atherosclerotic cardiovascular disease (CVD), stable CVD, unstable CVD, acute coronary syndrome (ACS), heart failure (HF), congestive HF, hospitalized HF, heart failure with reduced ejection fraction (EF), heart failure with preserved EF, ST-elevated myocardial infarction (STEMI), non-STEMI, or a disease, pathology, or condition related to or associated with heart or cardiac disease, familial or acquired, such as stroke, ischemic stroke, myocardial disease, peripheral artery disease, myocardial infarction, ischemic cardiomyopathy, non-ischemic cardiomyopathy, chemotherapy-induced cardiomyopathy, cerebrovascular disease, acute or chronic renal disease, and/or symptoms thereof. In an embodiment, the subject has had a myocardial infarction. In an embodiment, the subject has acute or chronic disease, for example, acute or chronic heart disease and/or associated coronary artery disease, e.g., stable coronary artery disease. In a particular embodiment, the subject has stable coronary artery disease (CAD).

In embodiments of any aspect of the methods described herein, the levels or concentrations of one or more of the PD markers HDL, (also called HDL-cholesterol (HDL-C) or HDL-esterified cholesterol (HDL-CE)), esterified cholesterol (CE), or apolipoprotein A1 (apoA1) increase (rapidly or over longer periods of time) following the administration of MEDI6012 to a subject, e.g., a subject who has heart disease and/or atherosclerotic disease. The levels of the PD markers may be measured or quantified in a biological sample obtained from a subject. A biological sample may include a body fluid sample, such as blood, serum, plasma, urine, saliva, and the like. Serum or plasma samples are particularly suitable for PD marker analyses in subjects who have been dosed with MEDI6012. By way of particular example, the practice of the methods described herein results in an increase in HDL-C and/or apoA1 levels in serum by approximately 50% within about 90 minutes, with an increase of at least 90%, or at least 95%, or at least 98%, or at least 100%, in at least HDL-C in serum by 6 hours, in a subject who has been administered a dose of LCAT (MEDI6012) according to the present methods. Moreover, apoA1 levels remain elevated for at least 7 days following intravenous infusion or subcutaneous administration of MEDI6012.

Example 1 herein describes a SAD study conducted to evaluate the administration of a single, ascending parenteral dose of the MEDI6012 rhLCAT enzyme to stable coronary artery disease (CAD) patients who were receiving statin therapy. The single dose of MEDI6012 was administered to the subjects by intravenous infusion or subcutaneous injection. The single infusion caused dose dependent increases in HDL cholesterol (HDL-C), HDL cholesteryl ester (HDL-CE), and total CE, which is consistent with a typical mechanism of action of the LCAT enzyme in the subjects. Based on the study, it was determined that a single dose of MEDI6012 caused dose-dependent increases in apolipoprotein A1 (apoA1) that peaked at doses between 80 mg and 240 mg.

In another embodiment, LCAT, such as rhLCAT or MEDI6012, administered to a subject in need at doses of 240 mg and above, e.g., 300 mg, 400 mg, 500 mg, 600 mg, 700 mg, or 800 mg, improve the function of HDL cholesterol particles, e.g., as determined by assessing cholesterol efflux capacity using methods known in the art. In another embodiment, multiple doses of lesser amounts of LCAT (e.g., 20-200 mg or 20-150 mg, or 20-100 mg) may also improve the function of HDL cholesterol particles in subjects administered rhLCAT or MEDI6012 in the described amounts.

In another embodiment, LCAT, such as rhLCAT or MEDI6012, administered to a subject in need at doses of less than or equal to 100 mg does not cause an accumulation of CE in LDL particles. Accordingly, the methods described herein which involve the administration of rhLCAT or MEDI6012 at doses in amounts of ≤100 mg provide treatment for the various cardiac, cardiac-related, cardiovascular and coronary artery diseases without accumulation of CE in LDL particles. The dosing methods described herein thus embrace long term dosing of LCAT (rhLCAT or MEDI6012) using various doses and dosing regimens, provided that LDL-CE accumulation is assessed and/or monitored as a dose limiting parameter in subjects undergoing LCAT treatment.

In yet another embodiment, the methods involving administering to subjects in need LCAT (rhLCAT or MEDI6012) at the described doses and dosing regimens results in a decrease of small LDL particles that are atherogenic. As noted herein, about a 40% reduction in small LDL particles was observed using rhLCAT or MEDI6012 at a dose of 80 mg, and about an 80% reduction in small LDL particles was observed using doses of rhLCAT or MEDI6012 in amounts of 240 mg and 800 mg.

Multiple Dose Treatment Methods Involving rhLCAT (MEDI6012)

In general, multiple ascending dose studies are conducted to elucidate the PK and pharmacodynamics (PD) of multiple doses of an administered compound or drug, usually in a clinical research unit (CRU). The dose levels and dosing intervals (i.e., the time(s) between consecutive doses) are selected as those that are predicted to be safe, based on the data obtained from the single dose studies. Biological samples are collected from the subjects and are analyzed to allow the determination of PK profiles and a better understanding of how the compound or drug is processed by the body. With multiple dosing, a key part of the PK analysis is to identify whether or not there is accumulation of the administered compound or drug. Similar to SAD studies, dose escalation in MAD studies proceeds according to the protocol, with strict safety and PK criteria being met. Dose levels and dosing frequency are selected to achieve therapeutic drug levels within the subject's systemic circulation, such that the drug levels are optimally maintained at steady state for several days to allow appropriate safety parameters to be monitored. It is usual for 2 to 3 dose levels to be studied, at and above the expected therapeutic dose level(s), to determine the ‘safety margin’ for repeated dose administration.

In an aspect, the present disclosure provides treatment methods as described herein involving the administration of multiple doses of the active, rhLCAT or MEDI6012, to treat a subject who has heart (cardiac) disease, cardiovascular disease and/or atherosclerotic disease, or who is in the throes of a myocardial infarction. Such dosing methods were developed and determined based on multiple ascending dose (MAD) studies carried out in a clinical study setting in which repeated doses of MEDI6012 were administered to subjects whose responses and levels of cholesterol and lipid metabolism components, products and by-products (e.g., pharmacodynamic (PD) markers) were also assessed following the administration of MEDI6012. As noted supra with regard to the SAD studies, such PD markers, which may be evaluated in a sample obtained from a subject prior to, during and/or following administration of MEDI6012 to the subject, include, without limitation, HDL-C; as well as additional lipids and lipoproteins whose levels are assessed and/or measured to fully understand and describe the effects of MEDI6012 on the cholesterol pathway. The assessed PD markers include, but are not limited to, total cholesterol (TC), free cholesterol (FC), cholesteryl ester (CE), HDL-esterified cholesterol (HDL-CE), HDL-unesterified cholesterol (HDL-UC), non-HDL-C, non-HDL-CE, non-HDL-UC, LDL-C (direct measure), VLDL-C, TG, apoB, apoA1, apoA1l, apoCIII, apoE. By way of example, immunoassays, such as an enzyme-linked immunosorbent assay (ELISA), may be used to characterize and quantify preβ1-HDL. Lipoprotein size and particle number for HDL, LDL, VLDL, etc., can be characterized by nuclear magnetic resonance (NMR), (LipoScience, Inc., Raleigh, N.C.).

In an aspect, the present disclosure provides a method of treating a subject afflicted with acute or chronic heart disease, cardiovascular disease, coronary artery disease (CAD), stable CAD, atherosclerosis, atherosclerotic cardiovascular disease (CVD), stable CVD, unstable CVD, acute coronary syndrome (ACS), heart failure (HF), congestive HF, hospitalized HF, heart failure with reduced ejection fraction (EF), heart failure with preserved EF, ST-elevated myocardial infarction (STEMI), non-STEMI, or a disease, pathology, or condition related to or associated with heart or cardiac disease, familial or acquired, such as stroke, ischemic stroke, myocardial disease, peripheral artery disease, myocardial infarction, ischemic cardiomyopathy, non-ischemic cardiomyopathy, chemotherapy-induced cardiomyopathy, cerebrovascular disease, acute or chronic renal disease, and/or symptoms thereof, in which the afflicted subject is administered more than one (repeated) doses of MEDI6012 during the course of treatment. In a particular embodiment, a method is provided in which an afflicted subject in need is administered three doses of MEDI6012 in which each administered dose of MEDI6012 is 25 mg to 2000 mg, or in which each administered dose of MEDI6012 is 30 mg to 800 mg, or in which each administered dose of MEDI6012 is 30 mg to 500 mg, or in which each administered dose of MEDI6012 is 30 mg to 300 mg, or in which each administered dose of MEDI6012 is 40 mg to 500 mg, or in which each administered dose of MEDI6012 is 40 mg to 300 mg. In embodiments, each administered dose of MEDI6012 is 25 mg, 30 mg, 40 mg, 50 mg, 60 mg, 70 mg, 80 mg, 90 mg, 100 mg, 120 mg, 130 mg, 140 mg, 150 mg, 160 mg, 180 mg, 190 mg, 200 mg, 220 mg, 230 mg, 240 mg, 250 mg, 260 mg, 280 mg, 290 mg, 300 mg, 320 mg, 330 mg, 340 mg, 350 mg, 360 mg, 380 mg, 390 mg, 400 mg, 420 mg, 430 mg, 440 mg, 450 mg, 460 mg, 480 mg, 490 mg, or 500 mg, including values therebetween. In an embodiment, the doses of MEDI6012 are administered intravenously, e.g., by intravenous (IV) infusion or by IV push. In an embodiment, the subject is administered 40 mg of MEDI6012 intravenously at three different time periods. In a particular embodiment, the method embraces a MEDI6012 dosing regimen in which a subject in need is intravenously administered a first dose of 40 mg of MEDI6012, a second 40 mg dose of MEDI6012 about one week following the first dose; and a third 40 mg dose of MEDI6012 about one week following the second dose, e.g., the subject is dosed with 40 mg MEDI6012 on days 1, 8 and 15. In another particular embodiment, the method embraces a MEDI6012 dosing regimen in which a subject in need is intravenously administered a first dose of 120 mg of MEDI6012, a second 120 mg dose of MEDI6012 about one week following the first dose; and a third 120 mg dose of MEDI6012 a week about one week following the second dose, e.g., the subject is dosed with 120 mg of MEDI6012 on days 1, 8 and 15. In another particular embodiment, the method embraces a MEDI6012 dosing regimen in which a subject in need is intravenously administered a first dose of 300 mg of MEDI6012, a second 300 mg dose of MEDI6012 about one week following the first dose; and a third 300 mg dose of MEDI6012 about one week following the second dose, e.g., the subject is dosed with 300 mg of MEDI6012 on days 1, 8 and 15. In an embodiment of any of the foregoing methods, MEDI6012 is administered to the subject intravenously, e.g., by intravenous (IV) infusion or by IV push. In an embodiment, MEDI6012 is administered intravenously by IV push over a time period of about or equal to 1-10 minutes, or about or equal to 1-5 minutes, or about or equal to 1-3 minutes, or about or equal to 1-2 minutes. In another embodiment, MEDI6012 is administered intravenously by IV infusion over a longer time period, such as over a time period of about or equal to 30 minutes to greater than 1 hour (e.g., 1-5 hours) or, more particularly, over a time period of about or equal to 1 hour. In a particular embodiment, the subject has stable CVD.

Methods of rhLCAT (MEDI6012) Administration Involving a Loading Dose

In accordance with another aspect of the present disclosure, statistical modeling data and predicted outcomes, as well as results obtained from the clinical studies involving rhLCAT or MEDI6012 dosing and dosing regimens as described herein, support an expected benefit and successful treatment resulting from methods involving the administration of a loading dose of MEDI6012 and subsequent doses (also called maintenance doses) of MEDI6012 to a subject having acute or chronic heart disease, cardiovascular disease, coronary artery disease (CAD), stable CAD, atherosclerosis, atherosclerotic cardiovascular disease (CVD), stable CVD, unstable CVD, acute coronary syndrome (ACS), heart failure (HF), congestive HF, hospitalized HF, heart failure with reduced ejection fraction (EF), heart failure with preserved EF, ST-elevated myocardial infarction (STEMI), non-STEMI, or a disease, pathology, or condition related to or associated with heart or cardiac disease, familial or acquired, such as stroke, ischemic stroke, myocardial disease, peripheral artery disease, myocardial infarction, ischemic cardiomyopathy, non-ischemic cardiomyopathy, chemotherapy-induced cardiomyopathy, cerebrovascular disease, acute or chronic renal disease, and/or symptoms thereof. Such methods involving a multiple dose approach, including a loading dose, for the administration of MEDI6012 treat the subject's heart (or heart-related) disease, cardiovascular disease, and the like, also increase PD biomarkers such as one or more of HDL, HDL-CE, CE and/or apoA1, while not causing an increase in levels of apoB, which is indicative that no serious, adverse, or detrimental effects are associated with any observed increase in LDL-C levels in a subject undergoing the multiple dose treatment methods.

The described methods which include a loading dose of LCAT, e.g., rhLCAT or MEDI6012, and more particularly, a loading dose administered to a subject as an IV push over about 1-3 minutes, allows for the treatment of diseases and conditions where time is of the essence. Unlike other drugs, rhLCAT or MEDI6012, administered according to the described doses and dose regimens, including a loading dose, can increase HDL-C levels within minutes. Therefore, the rapid action of rhLCAT or MEDI6012 in the described methods can quickly and effectively treat acute MI, stroke, and acute kidney injury. This feature of rhLCAT or MEDI6012 administration, among others, provides a highly favorable and crucial treatment that can be particularly effective for patients who need immediate, urgent treatment of acute disease, pathology, or injury, such as any of the foregoing.

In another aspect, a method is provided in which a subject is treated for acute or chronic heart disease, cardiovascular disease, coronary artery disease (CAD), stable CAD, atherosclerosis, atherosclerotic cardiovascular disease (CVD), stable CVD, unstable CVD, acute coronary syndrome (ACS), heart failure (HF), congestive HF, hospitalized HF, heart failure with reduced ejection fraction (EF), heart failure with preserved EF, ST-elevated myocardial infarction (STEMI), non-STEMI, or a disease, pathology, or condition related to or associated with heart or cardiac disease, familial or acquired, such as stroke, ischemic stroke, myocardial disease, peripheral artery disease, myocardial infarction, ischemic cardiomyopathy, non-ischemic cardiomyopathy, chemotherapy-induced cardiomyopathy, cerebrovascular disease, acute or chronic renal disease, and/or symptoms thereof, in which the method involves an IV dosing regimen that includes administering to a subject in need thereof three doses of MEDI6012, with the doses administered at predetermined intervals, such as weekly, or on days 1, 3 and 10, with day 1 being the first day of dosing. In a particular embodiment, the method involves administering to a subject with one or more of the above-noted heart or cardiovascular diseases or conditions (a subject in need) a loading or bolus dose of MEDI6012 in an amount of about or equal to 200-800 mg, or in an amount of about or equal to 250-600 mg, or in an amount of about or equal to 200-500 mg, or in an amount of about or equal to 250-500 mg, or in an amount of about or equal to 300-500 mg, or in an amount of about or equal to 300 mg.

In another particular embodiment, the method involves administering to a subject with one or more of the above-noted heart or cardiovascular diseases or conditions (a subject in need) a first (loading) dose of MEDI6012 in an amount of about or equal to 200-800 mg, or in an amount of about or equal to 250-600 mg, or in an amount of about or equal to 200-500 mg, or in an amount of about or equal to 250-500 mg, or in an amount of about or equal to 300-500 mg or in an amount of about or equal to 300 mg (Day 1 dose), followed by administering to the subject a second (or maintenance) dose of MEDI6012 in an amount of about or equal to 50-300 mg, or in an amount of about or equal to 100-250 mg, or in an amount of about or equal to 100-200 mg, or in an amount of about or equal to 100-150 mg, or in an amount of about or equal to 150 mg, at about or equal to 48 hours after the Day 1 dose (Day 3 dose), followed by administering to the subject a third (maintenance) dose of MEDI6012 in an amount of about or equal to 50-300 mg, or in an amount of about or equal to 100-200 mg, or in an amount of about or equal to 100-150 mg, or in an amount of about or equal to 100 mg at about 7-10 days, or at 7 days, or about a week, after the Day 3 dose (Day 10 dose). In an embodiment, the doses of MEDI6012 are administered to the subjects via intravenous administration. In an embodiment of the foregoing methods, at least the first dose of MEDI6012 is administered intravenously to the subject by IV push over a time period of about or equal to 1-5 minutes or over a time period of about or equal to 1-3 minutes, or over a time period of about or equal to 1-2 minutes. In some embodiments of the foregoing methods, all of the doses of MEDI6012 are administered to the subject by IV push over a time period of about or equal to 1-5 minutes or a time period of about or equal to 1-3 minutes. In an embodiment of the foregoing, the IV push, e.g., for loading or bolus dose administration, is administered to the subject over a time period of about or equal to 1 minute. In an embodiment of any of the foregoing methods, the times of rhLCAT or MEDI6012 dosing may be within about or equal to ±8 hours of the stated dosing times, time intervals, or time periods.

In a particular aspect, a method is provided in which a subject is treated for heart disease, cardiovascular disease, coronary artery disease (CAD), stable CAD, atherosclerosis, atherosclerotic cardiovascular disease (CVD), stable CVD, acute coronary syndrome (ACS), or a disease or condition related to or associated with heart or cardiac disease, such as stroke, ischemic stroke, myocardial disease, MI, and the like, and/or symptoms thereof, in which the method involves an intravenous IV dosing regimen that includes administering to a subject in need thereof a loading (first) dose of MEDI6012 in an amount of 300 mg (Day 1 dose), followed by administering to the subject a 150 mg dose of MEDI6012 (second or maintenance dose) at about or equal to 48 hours after the Day 1 dose (Day 3 dose), followed by administering to the subject a 100 mg dose of MEDI6012 (third or third maintenance dose) about 7 days after the Day 3 dose (Day 10 dose). In an embodiment, the doses of MEDI6012 are administered intravenously to the subjects. In an embodiment, one or more of the doses of MEDI6012 is administered to the subject via an intravenous (IV) push. In an embodiment of the foregoing method, one or more of the doses of MEDI6012 are administered to the subject by IV push infusion over a time period of about or equal to 1-10 minutes, or about or equal to 1-5 minutes, or about or equal to 1-3 minutes, or about or equal to 1-2 minutes, or about or equal to 1 minute. In a particular embodiment of the foregoing method, one or more of the doses of MEDI6012 are administered to the subject by IV push infusion over a time period of about or equal to 1-3 minutes. In a particular embodiment of the foregoing method, the subject has stable atherosclerotic CVD. In an embodiment of the foregoing method, the MEDI6012 dosing intervals may be within ±8 hours of the stated dosing times or time periods.

In another particular aspect, a method is provided in which a subject is treated for acute or chronic heart disease, cardiovascular disease, coronary artery disease (CAD), stable CAD, atherosclerosis, atherosclerotic cardiovascular disease (CVD), stable CVD, unstable CVD, acute coronary syndrome (ACS), heart failure (HF), congestive HF, hospitalized HF, heart failure with reduced ejection fraction (EF), heart failure with preserved EF, ST-elevated myocardial infarction (STEMI), non-STEMI, or a disease, pathology, or condition related to or associated with heart or cardiac disease, familial or acquired, such as stroke, ischemic stroke, myocardial disease, peripheral artery disease, myocardial infarction, ischemic cardiomyopathy, non-ischemic cardiomyopathy, chemotherapy-induced cardiomyopathy, cerebrovascular disease, acute or chronic renal disease, and/or symptoms thereof, in which the method involves a two-dose regimen comprising intravenously administering MEDI6012 to the subject at a dose of about or equal to 200-800 mg, or in an amount of about or equal to 250-600 mg, or in an amount of about or equal to 320-500 mg, or in an amount of about or equal to 300-500 mg, or in an amount of about or equal to 300 mg on Day 1, followed by intravenously administering MEDI6012 at a second dose of about or equal to 50-300 mg, or in an amount of about or equal to 100-250 mg, or in an amount of about or equal to 100-150 mg, or in an amount of about or equal to 150 mg at a predetermined time interval thereafter. In an embodiment, the second dose of MEDI6012 is administered from about or equal to 1-10 days following the Day 1 dose. In an embodiment, the second dose of MEDI6012 is administered on Day 3 (e.g., 48 hours±8 hours) following the Day 1 dose. In an embodiment, at least one of the doses of MEDI6012 is administered to the subject by IV push. In an embodiment, both the first and second doses of MEDI6012 are administered to the subject by IV push. In an embodiment, the IV push is administered over a time period of about or equal to 1-10 minutes, or about or equal to or over a time period of about or equal to 1-5 minutes, or about or equal to 1-3 minutes, or about or equal to 1-2 minutes, or about or equal to 1 minute. In a particular embodiment of the foregoing method, the subject has stable atherosclerotic CVD.

In yet another aspect, a method is provided in which a subject is treated for acute or chronic heart disease, cardiovascular disease, coronary artery disease (CAD), stable CAD, atherosclerosis, atherosclerotic cardiovascular disease (CVD), stable CVD, unstable CVD, acute coronary syndrome (ACS), heart failure (HF), congestive HF, hospitalized HF, heart failure with reduced ejection fraction (EF), heart failure with preserved EF, ST-elevated myocardial infarction (STEMI), non-STEMI, or a disease, pathology, or condition related to or associated with heart or cardiac disease, familial or acquired, such as stroke, ischemic stroke, myocardial disease, peripheral artery disease, myocardial infarction, ischemic cardiomyopathy, non-ischemic cardiomyopathy, chemotherapy-induced cardiomyopathy, cerebrovascular disease, acute or chronic renal disease, and/or symptoms thereof, in which the method involves intravenously administering six doses MEDI6012 to the subject at predetermined intervals.

In an embodiment, the method comprises a three dose regimen, in which rhLCAT or MEDI6012 is intravenously administered to a subject at a first dose of about or equal to 200-800 mg, or in an amount of about or equal to 250-600 mg, or in an amount of about or equal to 300-500 mg, or in an amount of about or equal to 300 mg on Day 1. The Day 1 dose is followed by intravenously administering MEDI6012 to the subject at a dose of about or equal to 50-300 mg, or in an amount of about or equal to 100-250 mg, or in an amount of about or equal to 100-150 mg, or in an amount of about or equal to 150 mg at about 1-5 days after the Day 1 dose, such as on Day 3 (e.g., 48 hours±8 hours) following the Day 1 dose, (called the “Day 3 dose”). The Day 3 dose is followed by intravenously administering MEDI6012 to the subject at a dose of about or equal to 100-250 mg, or in an amount of about or equal to 100-150 mg, or in an amount of about or equal to 100 mg at periodic intervals thereafter, such as weekly, or on days 10, 17, 24, and 31 following the Day 3 dose. In an embodiment, the dosing regimen encompasses three doses or six doses of rhLCAT or MEDI6012, including a first loading dose. In an embodiment, at least one of the doses of MEDI6012 is administered intravenously to the subject by IV push. In an embodiment, at least two of the doses of MEDI6012 are administered to the subject by IV push. In an embodiment, the Day 1 and Day 3 doses of MEDI6012 are administered to the subject by IV push. In an embodiment, the IV push is administered over a time period of about or equal to 1-10 minutes, or over a time period of about or equal to 1-5 minutes, or over a time period of about or equal to 1-3 minutes, or over a time period of about or equal to 1-2 minutes. In a particular embodiment, the IV push is administered over a time period of about or equal to 1-3 minutes or about or equal to 1-2 minutes.

In another particular embodiment, the method involves a six-dose regimen, which comprises administering MEDI6012 intravenously to a subject in need at a dose of 300 mg by IV push on Day 1, followed by administering MEDI6012 intravenously at a dose of 150 mg by IV push on Day 3 (48 hours±8 hours) following the Day 1 dose, followed by intravenously administering MEDI6012 at about weekly doses of 100 mg on Days 10, 17, 24, and 31 following the dose on Day 3. In embodiments, the doses administered to the subject on Days 10, 17, 24 and 31 are by IV push. In embodiments, the subject has cardiovascular disease, stable CAD, stable atherosclerotic CVD, or acute ST elevation myocardial infarction (STEMI).

In aspects of any of the foregoing methods, treatment of a subject as described results in an increase in blood, plasma, or serum levels of one or more of the markers HDL, HDL-C, HDL-CE, CE, and/or apoA1. In an embodiment, the increase in the marker levels is dose-dependent. In aspects of any of the foregoing methods, treatment of a subject as described results in a decrease in blood, plasma, or serum levels of apoB. In aspects of any of the foregoing methods, treatment of a subject as described results in little or no increase or significant alteration in blood, plasma, or serum levels of apoB. In an embodiment, any assessed increase in LDL or LDL-C marker levels is offset by a decrease, or little or no increase, in apoB levels. In an embodiment, the decrease in the marker levels is dose-dependent. In other embodiments of the methods, the administration of MEDI6012 at the doses and according to the dosing regimens described herein afford a cardio- and/or atheroprotective effect in a treated subject, for example, by reducing apoptosis of cardiomyocytes, reducing the levels of non-HDL associated cholesterol in serum, and causing excess cholesterol or LDL-C to be eliminated or removed from tissues and the body.

Example 2 herein describes a MAD clinical study conducted to evaluate the administration of multiple ascending parenteral doses of the MEDI6012 rhLCAT enzyme to stable atherosclerotic CVD patients. The doses of MEDI6012 were intravenously administered to the subjects. The results from the MAD studies in which repeated doses of MEDI6012 were administered to subjects demonstrated that the rate of the increases in HDL-C and/or apoA1 were dose-dependent, thus affording treatment and protective effects associated with the methods.

Other Treatment Methods Involving rhLCAT (MEDI6012) Administration

The present disclosure encompasses a method in which a dose of rhLCAT or MEDI6012 is advantageously provided to a patient who has a heart condition, pathology, or disease immediately after the patient presents at a hospital, emergency room, clinic, urgent healthcare facility, doctor's office, and the like. In accordance with the practice of the present methods, providing to the patient the doses of MEDI6012 and the dosing regimens involving MEDI6012 administration as described herein advantageously elevates the serum levels of HDL-C and/or apoA1 in the patient, and does not adversely affect the levels of serum apoB in the patient, thereby affording rapid myocardioprotective and atheroprotective effects that are also maintained over time, such as weeks. This is particularly in effect when dosing regimens involving follow-on doses of MEDI6012, e.g., maintenance doses, are provided to a patient after the administration of a first MEDI6012 dose, or when the dosing regimen involves a first loading dose of MEDI6012 as, for example, a bolus dose by IV push, followed by subsequent doses, e.g., maintenance doses, of MEDI6012 administered to the subject thereafter, as described herein.

In another aspect, the present disclosure provides a method of increasing the levels or amounts of one or more pharmacodynamic (PD) markers selected from HDL-C, CE, HDL-CE and/or apoA1, and/or decreasing or causing little or no change in the level of apoB, and/or decreasing the number of small atherogenic LDL particles in a subject who is afflicted with acute or chronic heart disease, cardiovascular disease, coronary artery disease (CAD), stable CAD, atherosclerosis, atherosclerotic cardiovascular disease (CVD), stable CVD, unstable CVD, acute coronary syndrome (ACS), heart failure (HF), congestive HF, hospitalized HF, heart failure with reduced ejection fraction (EF), heart failure with preserved EF, ST-elevated myocardial infarction (STEMI), non-STEMI, or a disease, pathology, or condition related to or associated with heart or cardiac disease, familial or acquired, such as stroke, ischemic stroke, myocardial disease, peripheral artery disease, myocardial infarction, ischemic cardiomyopathy, non-ischemic cardiomyopathy, chemotherapy-induced cardiomyopathy, cerebrovascular disease, acute or chronic renal disease, and/or symptoms thereof. The method includes administering to a subject a dose of MEDI6012 (rhLCAT) in an amount effective to result in an increase, a decrease, or little or no change in the above-noted PD markers. In embodiments, the method involves the intravenous or subcutaneous administration of one or more doses of MEDI6012, such as 24, 80, 240, 300, 600, or 800 mg, to the subject. In an embodiment, at least one dose of 80, 240, 300, or 800 mg of MEDI6012 is administered intravenously to the subject over a time period of 30 minutes to 1 hour. In a particular embodiment, the time period of intravenous administration of the MEDI6012 dose is 1 hour. In another embodiment, at least one dose of 80 or 600 mg of MEDI6012 is administered by SC injection to the subject. In other embodiments, the method involves the intravenous administration of multiple or repeated doses of MEDI6012, such two, three, or six doses of MEDI6012, to the subject. In a particular embodiment of the method, the first dose of MEDI6012 is a loading dose, which is administered in an amount of 200-500 mg, or more particularly, in an amount of 300 mg, followed by one or two doses of MEDI6012 (maintenance doses) administered at periodic intervals thereafter as described herein. In an embodiment, the loading dose is administered by IV push over a time period of about or equal to 1-5 minutes, or about or equal to 1-3 minutes, or about or equal to 1-2 minutes.

In another aspect, the present disclosure provides a method of conferring myocardial protection to a subject who is experiencing acute ST elevation myocardial infarction (STEMI) in which doses of MEDI6012 administered to the subject according to the doses and dosing regimens described herein increase HDL-C and/or HDL-CE levels so as to infuse HDL particles and/or apoA1 systemically and intracellularly, thereby resulting in a decrease in apoptotic events in myocardiocytes, for example.

### Combination Treatments

In another embodiment, a rhLCAT enzyme or MEDI6012 may be administered in conjunction with another drug, medication, or therapeutic agent or compound. In embodiments, rhLCAT or MEDI6012 is administered in conjunction with a statin drug, a proprotein convertase subtilisin/kexin type 9 (PCSK9) enzyme inhibitor (PCSK9i), other cholesterol-lowering drugs and medications, cardiac medications, and the like. In such a combination therapy, rhLCAT or MEDI6012 and another drug, medication, etc. may be administered together or separately, at the same time, sequentially, or at different times. In addition, other drugs or medications may be administered to the subject at the same time as, or at times different from, the administration of rhLCAT or MEDI6012. Without limitation, statins that may be administered include atorvastatin (LIPITOR), fluvastatin (LESCOL), lovastatin (MEVACOR, ALTOPREV), pitavastatin (LIVALO), pravastatin (PRAVACHOL), rosuvastatin (CRESTOR) and simvastatin (ZOCOR), evolocumab (REPATHA®), or alirocumab (PRALUENr). Other cholesterol-lowering drugs and medications may include fenofibrate (fenofibric acid (choline)), cholestyramine (QUESTRAN), Altocor, Cholestyramine Light, colestipol, niacin, Slo-Niacin, Niaspan, Caduet, Prevalite, Antara, Vytorin 10-80, Colestid, gemfibrozil, cholesterol absorption inhibitors, such as, ezetimibe (ZETIA) and ezetimibe-simvastatin, Triglide, Praluent, Lipofen, Repatha, Fibricor, Welchol, alirocumab and evolocumab.

A synergistic effect of a combination of therapies (e.g., a combination of rhLCAT or MEDI6012 and another cardio-therapeutic and/or cholesterol-lowering drug) may permit the use of lower dosages of one or more of the therapeutic agents and/or less frequent administration of the therapeutic agents to a subject with heart disease, coronary heart disease and/or artery disease. The ability to utilize lower dosages of therapeutic agents and/or to administer such therapeutic agents less frequently can reduce any potential toxicity that is associated with the administration of the therapies to a subject without reducing the efficacy of the therapies in the treatment of heart disease or coronary heart disease. In addition, a synergistic effect can result in improved efficacy of therapeutic agents in the management, treatment, or amelioration of heart disease or coronary heart disease. The synergistic effect of a combination of therapeutic agents can avoid or reduce adverse or unwanted side effects associated with the use of each therapy used singly (as monotherapy), e.g., at a higher dose.

In co-therapy, LCAT or MEDI6012 may be optionally included in the same pharmaceutical composition as the other drug or medication. Alternatively, LCAT or MEDI6012 may be in a separate pharmaceutical composition and may be administered at the same time or at a different time from one or more other drugs or medications. LCAT or MEDI6012, or a pharmaceutical composition comprising LCAT or MEDI6012, is suitable for administration prior to, simultaneously with, or following the administration of another drug or medication, or a pharmaceutical composition comprising the drug or medication. In certain instances, the administration of MEDI6012 to a subject overlaps with the time of administration of another or companion drug or medication provided separately or in a separate composition.

### Pharmaceutical Compositions and Formulations

The present disclosure encompasses the use of pharmaceutical compositions and formulations comprising the LCAT enzyme or MEDI6012 and one or more pharmaceutically acceptable excipients, carriers and/or diluents. In certain embodiments, the compositions may comprise one or more other biologically active agents (e.g., inhibitors of proteases).

Non-limiting examples of excipients, carriers and diluents include vehicles, liquids, buffers, isotonicity agents, additives, stabilizers, preservatives, solubilizers, surfactants, emulsifiers, wetting agents, adjuvants, etc. The compositions can contain liquids (e.g., water, ethanol); diluents of various buffer content (e.g., Tris-HCl, phosphate, acetate buffers, citrate buffers), pH and ionic strength; detergents and solubilizing agents (e.g., Polysorbate 20, Polysorbate 80); anti-oxidants (e.g., methionine, ascorbic acid, sodium metabisulfite); preservatives (e.g., Thimerosol, benzyl alcohol, m-cresol); and bulking substances (e.g., lactose, mannitol, sucrose). The use of excipients, diluents and carriers in the formulation of pharmaceutical compositions is known in the art, see, e.g., Remington's Pharmaceutical Sciences, 18th Edition, pages 1435-1712, Mack Publishing Co. (Easton, Pa. (1990)), which is incorporated herein by reference in its entirety.

By way of nonlimiting example, carriers can include diluents, vehicles and adjuvants, as well as implant carriers, and inert, non-toxic solid or liquid fillers and encapsulating materials that do not react with the active ingredient(s). Non-limiting examples of carriers include phosphate buffered saline, physiological saline, water, and emulsions (e.g., oil/water emulsions). A carrier can be a solvent or dispersing medium containing, e.g., ethanol, a polyol (e.g., glycerol, propylene glycol, liquid polyethylene glycol, and the like), a vegetable oil, and mixtures thereof.

Formulations comprising LCAT or MEDI6012 for parenteral administration can be prepared, for example, as liquid solutions or suspensions, as solid forms suitable for solubilization or suspension in a liquid medium prior to injection, or as emulsions. Sterile injectable solutions and suspensions can be formulated according to techniques known in the art using suitable diluents, carriers, solvents (e.g., buffered aqueous solution, Ringer's solution, isotonic sodium chloride solution), dispersing agents, wetting agents, emulsifying agents, suspending agents, and the like. Sterile fixed oils, fatty esters, polyols and/or other inactive ingredients can also be used. In addition, formulations for parenteral administration can include aqueous sterile injectable solutions, which can contain antioxidants, buffers, bacteriostats, and solutes that render the formulation isotonic with the blood of the intended subject and aqueous and nonaqueous sterile suspensions, which can contain suspending agents and thickening agents.

### Modes of Administration

In addition to the administration regimens described herein, rhLCAT or MEDI6012, or pharmaceutical compositions or formulations comprising rhLCAT or MEDI6012, can be administered to subjects by modes and routes that are suitable for administering and/or delivering a biological drug, such as a protein, to subject. In general, suitable biological delivery or administration methods embrace parenteral administration modes or routes. Such delivery methods include, without limitation, subcutaneous (SC) delivery, subcutaneous injection or infusion, intravenous (IV) delivery, e.g., intravenous infusion or injection or IV push. Other delivery and administration modes or regimens may include, without limitation, intra-articular, intra-arterial, intraperitoneal, intramuscular, intradermal, rectal, transdermal or intrathecal. In particular embodiments, MEDI6012 or rhLCAT is provided to a subject by intravenous administration, e.g., IV push or IV infusion. In another particular embodiment, MEDI6012 or rhLCAT is provided to a subject by subcutaneous injection, such as a single subcutaneous injection.

Recombinant human LCAT (rhLCAT) or MEDI6012 can be administered in a chronic treatment regimen. Recombinant human LCAT (rhLCAT) or MEDI6012 can be administered for a period of time as described herein, followed by a period of no treatment. A dosing regimen or cycle can also be repeated. In some embodiments, the treatment (e.g., administration of LCAT, MEDI6012 or rhLCAT) involves the administration of a loading dose as first treatment, followed by a second dose and/or one or more subsequent maintenance doses, e.g., for a time period comprising multiple days, e.g., day 1, day 3 and day 10 after the first or loading dose. Subsequent or maintenance doses may be administered at weekly intervals, e.g., 1 week, 2 weeks, 3 weeks, or longer, e.g., 4 weeks, 5 weeks, 6 weeks, 7 weeks, 8 weeks, 9 weeks, 10 weeks, 11 weeks, 12 weeks, or at monthly interval, or longer intervals, such as years, following initial, second or following doses.

It is also contemplated that MEDI6012 or rhLCAT can be administered by direct delivery, e.g., infusion or injection, at or near a site of disease, as practicable. It is also contemplated that MEDI6012 or rhLCAT can be administered by implantation of a depot at the target site of action, e.g., by cardiac catheter or stent. Alternative modes of administration or delivery of rhLCAT or MEDI6012 may include sublingual delivery under the tongue (e.g., sublingual tablet), inhalation (e.g., inhaler or aerosol spray), intranasal delivery, or transdermal delivery (e.g., by means of a patch on the skin). MEDI6012 or rhLCAT may also be orally administered if provided in a suitable form, e.g., microspheres, microcapsules, liposomes (uncharged or charged (e.g., cationic)), polymeric microparticles (e.g., polyamides, polylactide, polyglycolide, poly(lactide-glycolide)), microemulsions, etc. In addition, administration may be by osmotic pump (e.g., an Alzet pump) or mini-pump (e.g., an Alzet mini-osmotic pump), allowing for controlled, continuous and/or slow-release delivery of MEDI6012 or rhLCAT, or a pharmaceutical composition thereof, over a pre-determined period. The osmotic pump or mini-pump can also be implanted subcutaneously at or near a target site.

The present disclosure encompasses, unless otherwise indicated, conventional techniques of molecular biology (including any recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of LCAT polynucleotides and polypeptides as described herein, and, as such, may be considered in making and practicing the invention.

The following examples are set forth to provide those of ordinary skill in the art with a complete disclosure and description of how to make and use therapeutic methods of the invention, and are not intended to limit the scope of what the inventors regard as their invention.

### EXAMPLES

### Example 1—Single Ascending Dose (SAD) Studies of MEDI6012 in Subjects with Stable Coronary Artery Disease (CAD)

### Study Design Overview

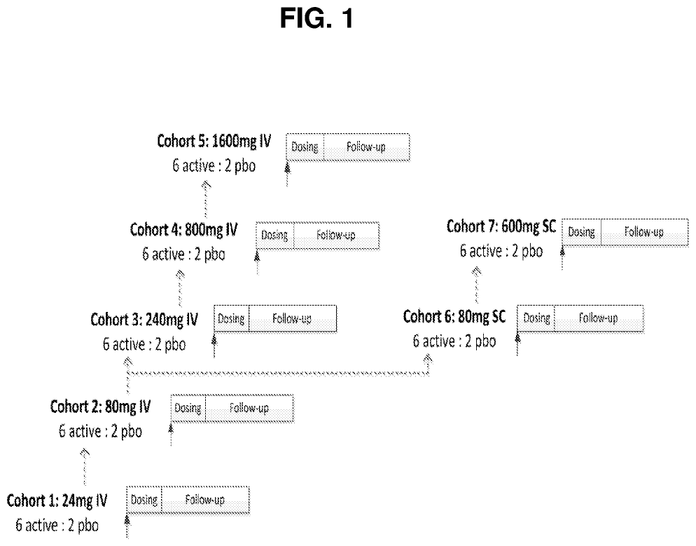

A Phase 2a randomized, double-blind (subject/investigator blinded; sponsor unblinded), placebo-controlled, dose-escalation study to evaluate the safety, PK/PD, and immunogenicity of single intravenous (IV/IV infusion) and subcutaneous (SC) doses of MEDI6012 in adult subjects with stable coronary artery disease (CAD) was conducted. A total of 48 subjects across 10 study sites in the United States of America were enrolled in the study to evaluate the following 4 dose levels (cohorts) of MEDI6012 via IV administration: 24 mg, 80 mg, 240 mg and 800 mg (Cohorts 1-4); and the following 2 dose levels (cohorts) of MEDI6012 administered via SC injection: 80 mg and 600 mg, shown as Cohorts 6 and 7, in FIG. 1. For each cohort, 8 subjects were randomized in a 6:2 ratio to receive MEDI6012 or placebo. For IV dose cohorts, MEDI6012 as the investigational product was administered as a 1-hour IV infusion in this study. For SC administration, MEDI6012 as investigational product was administered using single-use syringes containing up to 1 mL of volume per syringe. Subjects underwent a screening period of up to 28 days (if washout of a concomitant medication was required, a screening period of up to 42 days was allowed for such subjects). Subjects were admitted to the study center the evening prior to randomization and dose administration (Day −1) and remained at the study center until 7 days after the dose of the investigational product (Day 8). Subjects were followed through 28 days after receiving the dose of the investigational product (Day 29 visit). Subjects were encouraged to maintain a healthy lifestyle, including diet and exercise, during the study period.

### Statistical Analysis

### Sample Size:

The target subject population for the SAD studies was adult men or women, aged 40 through 75 years, with a history of documented stable CAD. A total of 48 subjects were studied. Each cohort had 8 subjects randomized in a 6:2 ratio to receive MEDI6012 or placebo. The sample size for this single-ascending dose study was empirically determined so as to provide adequate safety, tolerability, and PK/PD data to achieve study objectives. Eight subjects received placebo via IV administration and 4 subjects received placebo via SC administration. Assuming a common standard deviation of 280 and a two-sided alpha of 0.05, the current sample sizes provided >99% power to detect a difference of 1300 mg·hour/dL between each group of subjects receiving MEDI6012 versus placebo of the same route of administration for baseline adjusted HDL-C AUC_{0-96 h}.

The PD parameter of primary interest was the baseline-adjusted HDL-C area under the concentration curve (AUC) from 0 to 96 hours (AUC_{0-96 h}). AUC was calculated using the trapezoidal rule. Statistical comparison between treatment groups with placebo group combined was conducted using analysis of covariance (ANCOVA) by adjusting baseline HDL-C and treatment group. Other endpoints including AUC_{0-168 h} as well as AUC_{0-96 h} for HDL-C, TC, FC, CE, HDL-CE, HDL-UC, non-HDL-C, non-HDL-CE, non-HDL-UC, LDL-C (direct measure by standard laboratory test), and apoB for each of these were analyzed similarly to the primary PD endpoint.

Change and the percent change from baseline at each time point for each of the above lipids, lipoproteins, and apolipoproteins, as well as VLDL-C, TG, preβ1-HDL, apoA1, apoA1l, apoCIII, and apoE, were analyzed and compared using ANCOVA by adjusting baseline and treatment group with placebo group combined. Descriptive statistics were provided by treatment group for maximal response and time of maximal response for each of these as well. ADA incidence rate and titer was tabulated for each treatment group. Samples confirmed positive for ADA were tested and analyzed for neutralizing antibody (nAb) titer and summarized similarly. Non-compartmental analysis was performed for MEDI6012-treated subjects. MEDI6012 mass and activity concentration-time profiles were summarized by dose cohort. The PK parameters reported included Cₘₐₓ, Tₘₐₓ, AUC, SC bioavailability, and terminal half-life (t_{1/2}).

### Primary and Secondary Study Objectives

The primary safety objective of the study was to assess the safety of MEDI6012 following single-ascending doses in subjects with stable CAD. The primary pharmacodynamic (PD) objective was to measure the dose response for HDL-C following administration of MEDI6012. The endpoint for the primary PD objective involved baseline-adjusted area under the curve from time 0 to 96 hours (AUC_{0-96 h}) post dose for HDL-C. The secondary objectives involved measurement of the dose response for other key PD biomarkers following administration of MEDI6012; establishment of the PK profile of MEDI6012 administered IV and SC; determining the relationship between MEDI6012 and preβ1-HDL substrate; and assessing the immunogenicity potential of MEDI6012. The endpoints for the secondary objectives involved assessment of serum concentration of other key lipids, lipoproteins, and apolipoproteins: total cholesterol (TC), free cholesterol (FC), cholesteryl ester (CE), high-density lipoprotein-cholesteryl ester (HDL-CE), high-density lipoprotein-unesterified cholesterol (HDL-UC), non-HDL-C, non-HDL-CE, non-HDL-UC, LDL-C (direct measure), very low-density lipoprotein-cholesterol (VLDL-C), triglycerides (TG), apoplipoprotein B (apoB), apolipoprotein A1 (apoA1), apolipoprotein A1l (apoAII), apolipoprotein CIII (apoCIII) and apolipoprotein E (apoE); measurement of serum concentration for MEDI6012 mass; assessment of Preβ1-HDL particles; and ADA and nAb titers.

Exploratory objectives included the exploration of lipoprotein size and particle number; verification of LDL-C levels using alternative methodologies; and evaluation of the effect of MEDI6012 on the capacity of plasma from treated subjects to support cholesterol efflux. The exploratory objective endpoints included measurement of serum concentration for MEDI6012 activity; HDL, LDL, and VLDL particle size and particle number; measurement of LDL-C by ultracentrifugation (β-quant) and Friedewald equation; and determination of global cholesterol efflux with LCAT esterification activity assay in HDL, using known protocols, such as, for example, as described in Shamburek, R. D. et al., 2016, Circulation Research, 118:73-82; doi: 10.1161/CIRCRESAHA.115.306223, 2015.

### Study Population

The study population consisted of adults with a history of documented CAD that was clinically stable. This population struck the best balance to permit safety, PK, and PD assessment of MEDI6012 in subjects with established atherosclerosis, the target population for subsequent clinical development, but who were clinically stable (lower safety risk) with less fluctuation in biomarker levels to enable robust PK/PD decisions. Subjects with unstable CAD as well as unstable or progressive angina were excluded. A healthy subject population was not selected for this study. Use of stable CAD patients in this study allowed for the acquisition of lipid profile data in patients with lipid profiles that were more likely to be consistent with an ACS population than a healthy subject population. In addition, there were no safety signals identified in the prior study or in MEDI6012 preclinical studies to suggest a safety concern for evaluating single dose administration of MEDI6012 in stable CAD patients.

Subjects were required to be on a stable statin regimen as standard of care (SoC), with LDL-C levels ≤150 mg/dL at screening, to avoid enrolling subjects with genetically low LDL receptor concentration (and thus high or very high baseline LDL-C) and to provide a more homogeneous population against which to evaluate the lipid/lipoprotein changes of interest. Similarly, subjects with high baseline HDL-C values (>60 mg/dL for men, >65 mg/dL for women) were excluded to provide consistency among the study subjects for the upward movement of HDL-C levels that facilitated dose selection.

### Rationale for Primary Endpoints, Key PD Endpoints, PK Endpoints and Immunogenicity Endpoints

The primary PD endpoint was HDL-C, which was analyzed as baseline-adjusted AUC_{0-96 h}. Since HDL is a substrate for rhLCAT, it was expected that the effectiveness of MEDI6012 would correlate with changes in HDL-C levels. This is supported by the MEDI6012 cynomolgus monkey toxicology study (normal animals with intact endogenous rhLCAT and high levels of HDL-C) that showed a robust and transient increases in HDL-C following MEDI6012 infusion. HDL-C is a more consistent/less variable assay endpoint than CE; thus, CE was selected as a secondary PD endpoint for the study.

Lipoproteins and lipid panel components were selected because movement of these markers as a result of MEDI6012 dosing provided supporting evidence of increased activity on the RCT system. TC represents the sum of unesterified and esterified cholesterol on all plasma lipoproteins. HDL-C represents the amount of cholesterol present in HDL particles, which can be further categorized as HDL-UC and HDL-CE fractions. Through its enzymatic activity, MEDI6012 was expected to result in increases in HDL-C, the primary PD biomarker. TC, CE, and LDL-C levels are markers that further assess the effects of rhLCAT on RCT and were therefore identified as secondary PD endpoints.

Serum concentration of LCAT (mass) was used to characterize MEDI6012 exposure and related to toxicological exposures. The PK could also be used to develop dose-exposure-PD response relationships to help inform dose selection for future clinical trials. Plasma LCAT activity provided an alternative measure to LCAT mass in establishing the relationship between PK and PD for MEDI6012 in a stable CAD population.

Formation of ADA against MEDI6012 had the potential to impact the safety, PK, and/or PD of MEDI6012 and/or endogenous LCAT. The ADA potential of this compound was assessed, and formation of any nAb was also characterized.

### Rationale for Exploratory PD Endpoints

Lipoprotein Particle Size and Particle Number:

Preβ1-HDL (the first HDL particle involved in RCT) is a small, lipid-poor, discoid particle that accepts cholesterol at peripheral cells through the binding of ABCA1 to apoAI. The resulting complex is then converted to a larger particle, preβ2-HDL, by incorporation of additional cholesterol. The cholesterol is esterified via the action of LCAT, which converts the particle into the larger, spherical α3-HDL particle, which is then further converted to α2-HDL, and then to al-HDL as it acquires more cholesterol. The esterification reaction is thought to help maintain a concentration gradient that drives the movement of cholesterol to HDL, thus increasing the ability of HDL to accept more cholesterol (Fielding et al, 1995a, Journal of Lipid Research, 36(2):211-28). The CE in mature HDL is eliminated either by direct selective uptake by the liver (minor route) or by transfer to apoB-containing lipoproteins via the action of CETP; the apoB-containing lipoproteins are then cleared through the hepatic low-density lipoprotein receptor (LDLr) pathway (major route). It was expected that the transfer of CE to the apoB-containing lipoproteins with the eventual maturation of larger more cholesterol-rich LDL particles would facilitate uptake through the LDLr. It is important that this occurs without an increase in particle number. FC efflux from cells, esterification of the FC by LCAT, and uptake of CE by the liver are collectively an important first step in RCT (Fielding et al, 1995b, Biochemistry, 34(44):14288-92; Miller, 1990, Baillieres Clin Endocrinol Metab., 4(4):807-32; Tall et al, 2008, Cell Metab., 7(5):365-75). The protocol-specified endpoints were chosen to provide information on the in vivo activity of MEDI6012 on HDL maturation and to provide insights into its mechanism of action.

Verification of LDL-C by Ultracentrifugation:

Turner et al. (2015. https://wwwmedpacecom/PDF/Posters/PosterD1_MRL-2015pdf) compared calculated LDL-C (by the Friedewald equation), the direct/homogenous LDL-C assay (direct measure by a standard laboratory test), and the “gold standard” preparative ultracentrifugation (β-quant) assay. Formulas for calculating LDL-C and “direct” LDL-C measurement showed significant and clinically meaningful differences when true LDL-C was <70 mg/dL, and even moderate increases in triglycerides had major effects on measurements. The “gold standard” ultracentrifugation method (exploratory) was therefore included in this trial to provide comparison of LDL-C direct (secondary measure) and calculated LDL-C (exploratory) to facilitate selection of the optimal LDL-C measure for the LCAT mechanism and patient population.

Cholesterol Efflux and LCAT Esterification Assay:

These are novel biomarkers of upregulated RCT and HDL functionality (going beyond changes in HDL-C and particle size), measuring the ability of MEDI6012 to up-regulate cholesterol efflux from peripheral tissues.

### Materials and Methods

Investigational Product/Drug Product:

MEDI6012, the investigational product/drug product administered in the studies and methods described herein, was manufactured by MedImmune, LLC and supplied as a lyophilized powder (100 mg per mL upon reconstitution with sterile water for injection) in a buffer containing 10 mM sodium phosphate, 300 mM sucrose, 0.06% weight by volume (w/v) poloxamer-188 at pH 7.2. MEDI6012 was provided as a sterile white to off-white lyophilized powder (50 mg/vial, nominal). Upon reconstitution with 0.6 mL sterile Water for Injection (sWFI), MEDI6012 is a colorless to yellow solution.

Placebo used in the studies was manufactured by MedImmune, LLC and was supplied as 10 mL solution containing 10 mM sodium phosphate, 300 mM sucrose, 0.06% (w/v) poloxamer-188 at pH 7.2. Placebo was provided as a sterile colorless to slightly yellow solution.

In addition to the investigational product, an intravenous bag protectant (IVBP) solution was supplied to prevent adsorption of the MEDI6012 product to the IV infusion system. The IVBP was stored at 2-8° C. (36-46° F.). The IVBP was supplied for use as a 10 mL solution containing 10 mM sodium phosphate, 300 mM sucrose, 0.06% (w/v) and poloxamer-188 at pH 7.2. The IVBP was supplied in 10R vials as a colorless to slightly yellow, clear to slightly opalescent liquid. Lyophilized MEDI6012 was not reconstituted with the IVBP solution.

IV Administration:

Each IV dose was delivered to subjects as an admixture of reconstituted MEDI6012 and IVBP or placebo plus IVBP, in a 0.9% saline IV bag. The IVBP was used for IV doses only. For all IV cohorts, IVBP was used to precondition the IV bag prior to the addition of the MEDI6012 drug or the placebo dose. For each dose, the lyophilized MEDI6012 drug product vials, liquid placebo vials, liquid IVBP vials were inspected, and 0.9% (weight by volume, w/v) saline was added to the IV bag prior to preparation of active drug product dose or placebo dose. For active drug product arms, only the required number of vials per dose of MEDI6012 was reconstituted.

No incompatibilities between MEDI6012 and plastics (polyolefin without di-2-ethylhexyl phthalate (DEHP) bags and polypropylene syringes) were observed when used in conjunction with the IVBP. Polyethylene/polyvinylchloride (PE/PVC) and PVC DEHP-free IV extension lines were acceptable. Lines contained either 0.22 or 0.2 nm in-line filter. The in-line filter was typically made of polyethersulfone (PES). Lines containing cellulose-based filters was not used with MEDI6012, as these were not tested.

The MEDI6012 product, placebo and IVBP did not contain preservatives; therefore, any unused portion was discarded. The total in-use storage time from needle puncture of the first investigational product vial(s) to the start of IV administration should not exceed 4 hours at room temperature or 24 hours at 2° C. to 8° C. (36° F. to 46° F.). If storage time exceeded these limits, a new dose was prepared from a new investigational product vial(s) and IVBP vial. If a prepared dose was stored at 2° C. to 8° C. (36° F. to 46° F.), the vial was equilibrated to room temperature and inspected prior to IV administration to ensure that the solution of MEDI6012 to be dosed was clear.

SC Administration:

Each SC dose was delivered as reconstituted MEDI6012 or placebo. No incompatibilities were observed between MEDI6012 and polycarbonate/polypropylene syringes used for SC administration. The MEDI6012 drug and placebo did not contain preservatives and any unused portion was discarded. The total in-use storage time from needle puncture of the first investigational product vial(s) to start of SC administration did not exceed 4 hours at room temperature. If storage time exceeded these limits, a new SC dose was prepared from a new vial(s)

Preparation and Administration of MEDI6012 or Placebo for IV Administration:

The admixture for doses from 24 mg to 1600 mg was prepared in a 50 mL polyolefin 0.9% saline IV bag containing IVBP using a single step dilution. While the 1600 mg dose was proposed for testing in the study, this dose was not tested in subjects, because of the efficacy determined using the lower doses of rhLCAT or MEDI6012. The prepared dose was delivered using a PVC (DEHP-free) IV administration set with a 0.22 or 0.2-nm PES filter. For IV administration by IV infusion, the administration components, including filter, of the IV bag were attached and the administration line was primed immediately prior to infusion. The dose of MEDI6012 was administered as an IV infusion over approximately 60 minutes (±5 minutes). Following the complete infusion of the IV bag, a flush of the IV administration set was performed by adding up to 30 mL of 0.9% saline (or equivalent corresponding to the hold-up volume of the extension set) to the IV bag to ensure that the complete dose of MEDI6012 was delivered.

Preparation and Administration of MEDI6012 or Placebo for SC Injection:

Each subcutaneous (SC) dose of MEDI6012 was by injection, delivered by syringe. The MEDI6012 drug or placebo could be pooled in an appropriately-sized syringe (polycarbonate/polypropylene) or sterile glass vial (e.g., 10 mL) and dosed based on the delivery volume. The dose was delivered using a 27 G, 0.5 inch syringe needle. In addition, the IVBP was not used in the preparation of SC doses.

Treatment and Monitoring of Dose Administration

In the study, the day of MEDI6012 dosing was considered Day 1. On the day of the dose, following an overnight fast by the subject for a minimum of 6 hours, the MEDI6012 investigational product was administered as soon as was practicable after the subject arose. For IV infusion, the MEDI6012 product was administered over a period of approximately 60 minutes (±5 minutes). For SC injection, the MEDI6012 product was administered in the lower abdomen utilizing a 27 G, 0.5 inch needle. Where multiple injections were required to administer the dose of the drug product, separate injection sites were used. The individual injections were administered in the abdomen and spaced at least 3 cm apart. For subjects who also took insulin or other concomitant medications via SC administration, injection of those medications were at a location different from that of the MEDI6012 drug product administration. The skin surface of the abdomen was prepared with an alcohol wipe and allowed to air dry.

The skin was pinched to isolate SC tissue from the muscle. The needle was inserted at a 90-degree angle approximately halfway into the SC tissue. The MEDI6012 investigational product was slowly injected (at least 5-second duration was recommended per 1-mL syringe) into the SC tissue using gentle pressure. The area was not massaged after injection.

Vital signs, ECG assessments, and telemetry (for IV administration) were performed before and after dose administration. As with any exogenous protein, allergic reactions to dose administration may be possible. Therefore, appropriate drugs and medical equipment to treat acute anaphylactic reactions were immediately available, and study personnel were trained to recognize and treat anaphylaxis.

During the study period, subjects continued to take their prescribed statin therapy at their regular prescribed dose, and any other medication(s), e.g., blood pressure or heart medication, prescribed for their disease, such as CAD.

Methods for Assigning Treatment Groups:

An interactive voice/web response system (IXRS) was used for randomization of subjects to a treatment group and assignment of blinded investigational product kit numbers. A subject was considered randomized into the study when the investigator notified the IXRS that the subject met eligibility criteria and the IXRS provided the assignment of blinded investigational product kit numbers to the subject.

For each cohort, 8 subjects were randomized in a 6:2 ratio to receive MEDI6012 or placebo. A sentinel dosing approach was planned for each cohort. For sentinel dosing, 2 subjects were randomized in a 1:1 ratio to receive MEDI6012 or placebo first. A time lag of ≥24 hours occurred before the remaining subjects in the cohort were dosed.

The investigational product was administered within 24 hours after randomization. If there was a delay in the administration of investigational product such that it was not administered within the specified timeframe, the medical monitor was notified immediately.

Permitted Concomitant Medications:

It was anticipated that subjects enrolled in the study and who had established atherosclerotic CVD would be managed per current treatment guidelines (e.g., AHA/ACCF Secondary Prevention and Risk Reduction Therapy for Patients with Coronary and Other Atherosclerotic Vascular Disease, 2011 and ACC/AHA Blood Cholesterol Guideline, 2013) and would have been receiving a range of cardio-protective medications. Subjects were required to adhere to their current regimen from screening through the end of the study. Investigators prescribed concomitant medications or treatments deemed necessary to provide adequate supportive care except for “excluded” medications as described below. Specifically, subjects continued to take their regular prescribed dose of statin and blood pressure medication and received full supportive care during the study, including transfusions of blood and blood products, and treatment with antibiotics, anti-emetics, anti-diarrheals, and analgesics, and other care as deemed appropriate, and in accordance with their institutional guidelines.

Concomitant medications, including over-the-counter medications, herbal supplements, and vitamins that may affect control of lipids (except for statins) were prohibited from screening through the final study visit. Subjects were instructed not to take any medications, including over-the-counter products, without first consulting with the investigator. Due to their effect on lipids, systemic corticosteroids within 28 days prior to screening and throughout the study were also prohibited, except if needed to treat a generalized allergic reaction, anaphylaxis as defined by the study guidelines, or other serious medical condition. Inhaled, intranasal, topical, ophthalmic and intra-articular corticosteroids were permitted. The use of systemic corticosteroid required discussion with and permission by the medical monitor.

### Statistical Evaluations

General Considerations:

Data were provided in listings sorted by cohort, treatment group and subject number. Tabular summaries were presented by treatment group with placebo group combined (and separately by IV and SC routes) when appropriate. Categorical data were summarized by the number and percentage of subjects in each category. Continuous variables were summarized by descriptive statistics, including mean, standard deviation, median, minimum, and maximum. Baseline values were defined as the last valid assessment prior to the first administration of investigational product.

Definition of Analysis Population:

The As-treated Population included all subjects who had received any study investigational product (MEDI6012). Subjects were analyzed according to the treatment they actually received. The PK population included all subjects in the As-treated Population who had at least one detectable LCAT serum concentration measurement.

Sample Size and Power Calculations:

The study was designed to include a total of 48 subjects for enrollment. Each cohort had 8 subjects randomized in a 6:2 ratio to receive MEDI6012 or placebo. (FIG. 1). The sample size for this single-ascending dose study was empirically determined and was designed to provide adequate safety, tolerability, and PK/PD data to achieve study objectives while exposing as few subjects as possible to the investigational product and study procedures.

In the study, 8 subjects received placebo via IV administration and 4 subjects received placebo via SC administration. Assuming a common standard deviation of 280 and a two-sided alpha of 0.05, the current sample sizes provided >99% power to detect a difference of 1300 mg·hour/dL between each MEDI6012 group versus placebo of the same route of administration for baseline adjusted HDL-C AUC₀_96 h.

### Study Results

The SAD study met its primary PD endpoint and achieved dose-dependent increases in HDL-C at lower than expected doses. A single IV dose of MEDI6012 administered to subjects in an amount of 24 to 800 mg demonstrated dose-dependent increases in high-density lipoprotein-cholesterol (HDL-C), high-density lipoprotein-cholesteryl ester (HDL-CE), and CE consistent with the mechanism of action of the LCAT enzyme. As shown in the below Table 1, a statistically significant increase with regard to baseline-adjusted AUC (0-96h) in HDL-C was observed across all of the IV treatment groups (i.e., 80 mg, 240 mg and 800 mg doses of MEDI6012), except for the group that had received MEDI6012 at a dose of 24 mg. The AUC (0-96 h) increase in HDL-C was determined to be dose-dependent.

TABLE 1
Baseline-adjusted AUC (0-96 h) in HDL-C; As-treated Population (IV Group)

| | | MEDI6012 | MEDI6012 | MEDI6012 | MEDI6012 | MEDI6012 |
|---|---|---|---|---|---|---|
| | Placebo IV | 24 mg IV | 80 mg IV | 240 mg IV | 800 mg IV | IV Total |
| | (N = 8) | (N = 6) | (N = 6) | (N = 6) | (N = 6) | (N = 24) |
| N | 8 | 6 | 5 | 6 | 6 | 23 |
| Mean | −49.1 | 728.3 | 1639.9 | 3035 | 5318.3 | 2725.6 |
| (SD) | (120.0) | (334.2) | (631.7) | (439.1) | (1674.3) | (1998.5) |
| Median | −37.2 | 749.8 | 1724.9 | 3221.8 | 5214.4 | 2394.3 |
| LS mean | −55.4 | 668.2 | 1607.8 | 3147.2 | 5301.3 | 2726.9 |
| P-value | | 0.095 | <0.001 | <0.001 | <0.001 | <0.001 |

As shown in the below Table 2, a statistically significant increase with regard to baseline-adjusted AUC (0-168h) in HDL-C was observed across all of the IV treatment groups (i.e., 80 mg, 240 mg and 800 mg doses of MEDI6012), except for the group that had received MEDI6012 at a dose of 24 mg. The AUC (0-168h) increase in HDL-C appeared to be dose-dependent.

TABLE 2
Baseline-adjusted AUC (0-168 h) in HDL-C; As-treated Population (IV Group)

| | | MEDI6012 | MEDI6012 | MEDI6012 | MEDI6012 | MEDI6012 |
|---|---|---|---|---|---|---|
| | Placebo IV | 24 mg IV | 80 mg IV | 240 mg IV | 800 mg IV | IV Total |
| | (N = 8) | (N = 6) | (N = 6) | (N = 6) | (N = 6) | (N = 24) |
| N | 8 | 6 | 5 | 6 | 6 | 23 |
| Mean | −40.8 | 1084.7 | 2216.1 | 4518.9 | 7838.9 | 3988.5 |
| (SD) | (394.4) | (397.3) | (1280.4) | (807.1) | (2956.5) | (3098.7) |
| Median | −67.5 | 1125.1 | 2441.7 | 4642.5 | 7264 | 3834.3 |
| LS mean | −51.9 | 978.5 | 2159.4 | 4717.1 | 7808.8 | 3991 |
| P-value | | 0.182 | 0.01 | <0.001 | <0.001 | 0.001 |

For the subcutaneous (SC) MEDI6012 treatment population, a statistically significant increase with regard to baseline-adjusted AUC (0-96h) in HDL-C was observed in the treatment group of subjects who had received 600 mg of MEDI6012 by SC administration. This is shown in Table 3 below:

TABLE 3
Baseline-adjusted AUC (0-96 h) in
HDL-C; As-treated Population (SC Group)

| | | MEDI6012 | MEDI6012 | MEDI6012 |
|---|---|---|---|---|
| | Placebo SC | 80 mg SC | 600 mg SC | SC Total |
| | (N = 4) | (N = 6) | (N = 6) | (N = 12) |
| N | 4 | 5 | 5 | 10 |
| Mean | −113.5 (289.4) | 422.4 (395.7) | 2844.7 (1219.1) | 1633.5 (1536.2) |
| (SD) | | | | |
| Median | −65.5 | 515.2 | 3029.9 | 1076.2 |
| LS | −43.7 | 403.6 | 2807.7 | 1595.6 |
| mean | | | | |
| P-value | | 0.440 | <0.001 | 0.085 |

In addition, a statistically significant increase with regard to baseline-adjusted AUC (0-168h) in HDL-C was observed in the in the treatment group of subjects who had received 600 mg of MEDI6012 by SC administration, as shown in Table 4 below:

TABLE 4
Baseline-adjusted AUC (0-168 h) in
HDL-C; As-treated Population (SC Group)

| | | MEDI6012 | MEDI6012 | MEDI6012 |
|---|---|---|---|---|
| | Placebo SC | 80 mg SC | 600 mg SC | SC Total |
| | (N = 4) | (N = 6) | (N = 6) | (N = 12) |
| N | 4 | 5 | 5 | 10 |
| Mean | −168.1 (620.3) | 721.1 (714.2) | 5250.1 (2175.6) | 2985.6 (2833.4) |
| (SD) | | | | |
| Median | −221.8 | 863.5 | 5629.4 | 1998.6 |
| LS | −38 | 686.1 | 5181.0 | 2914.8 |
| mean | | | | |
| P-value | | 0.486 | <0.001 | 0.092 |

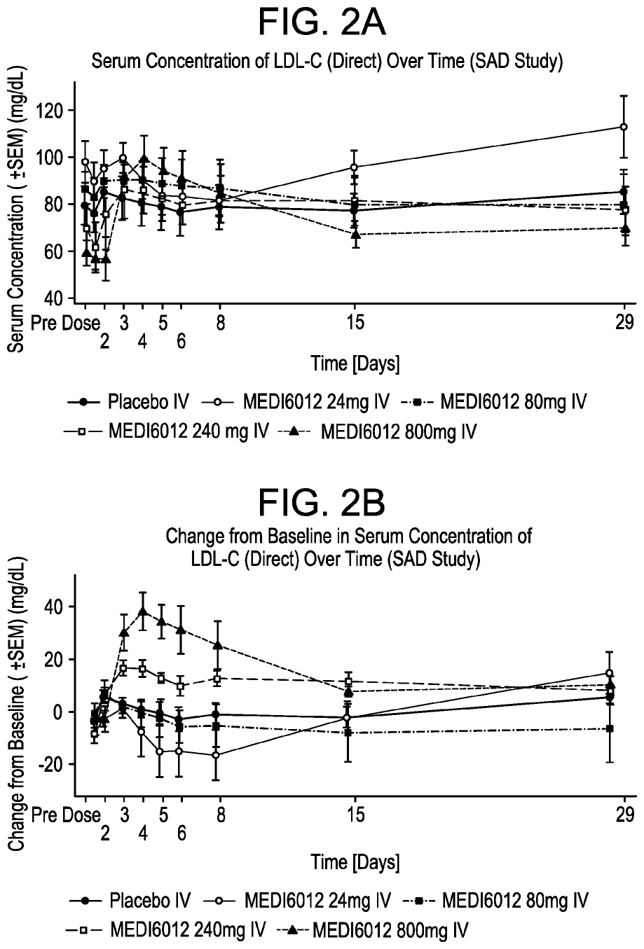

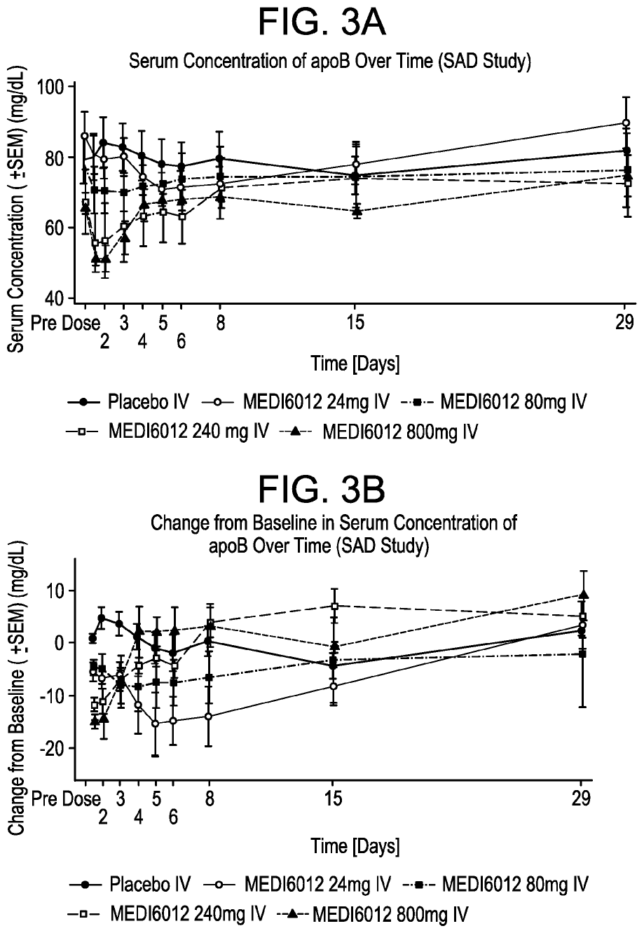

The intravenous administration of MEDI6012 to study subjects resulted in a dose-dependent increase in LDL-C levels in subjects' serum (FIGS. 2A and 2B), but also resulted in statistically significant decreases in apoB across all of the IV dose levels, i.e., 24, 80, 240, 800 mg, (FIGS. 3A and 3B). ApoB has been reported to be a better predictor of risk of CHD than LDL-C in both men and women, and the number of atherogenic particles, e.g., apoB, can serve as a more important indicator of risk than the amount of cholesterol (LDL-C) transported in these particles. (reviewed in Vaverkova, H., 2011, Clin Lipidology, 6(1):35-48; Sniderman, A D et al., 2003, Lancet, 361:777-780). Because all potentially atherogenic lipoprotein particles contain only one molecule of apoB and various amounts of cholesterol, apoB serves as a better marker of atherogenic lipoprotein particle numbers than LDL-C. Therefore, the finding of significant decreases in apoB following the administration of the various doses and the dose regimens of MEDI6012 as described herein demonstrates that MEDI6012 provides significant advantages for beneficial and protective treatment of subjects with cardiovascular disease despite the observation of an increase in LDL-C.

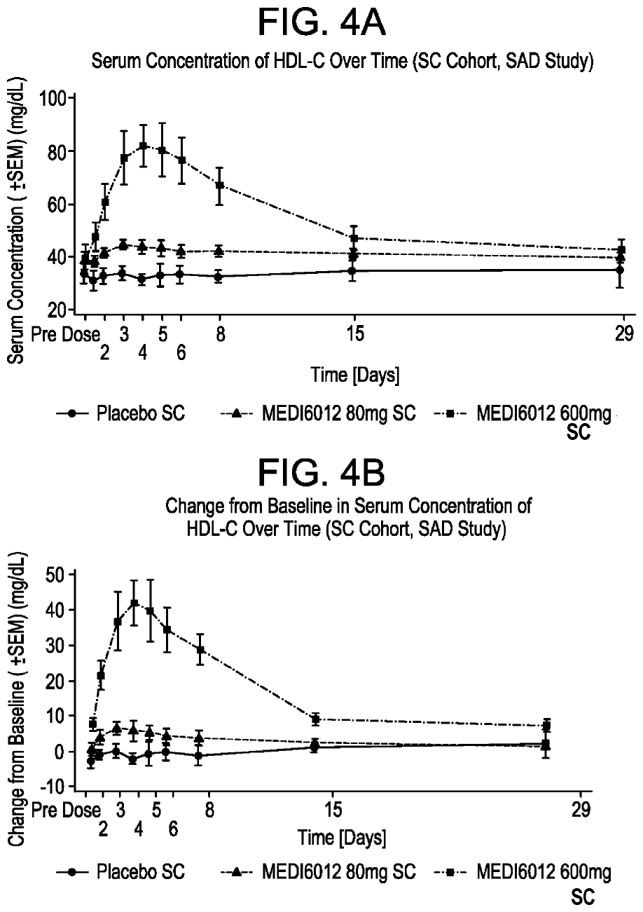

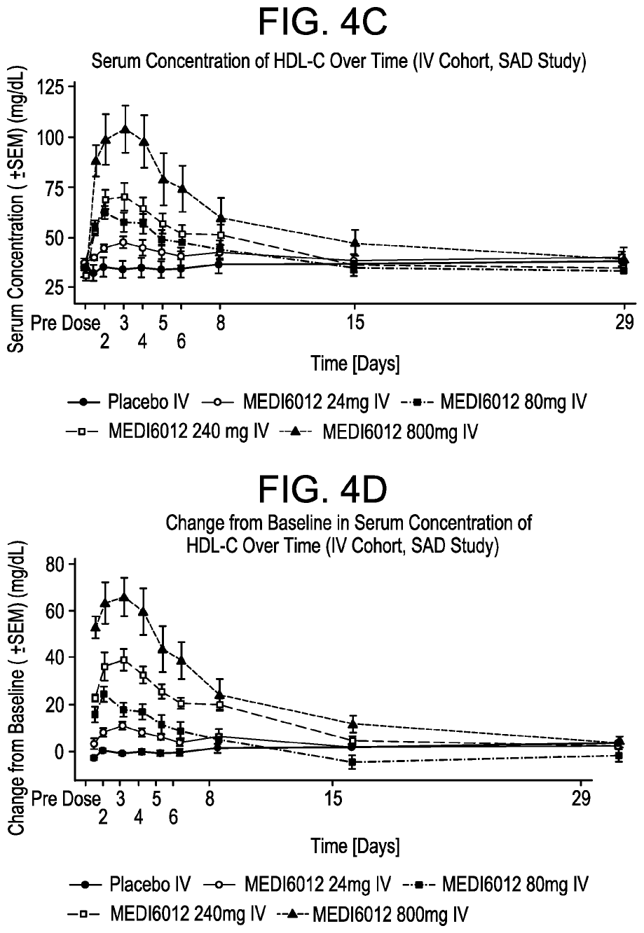

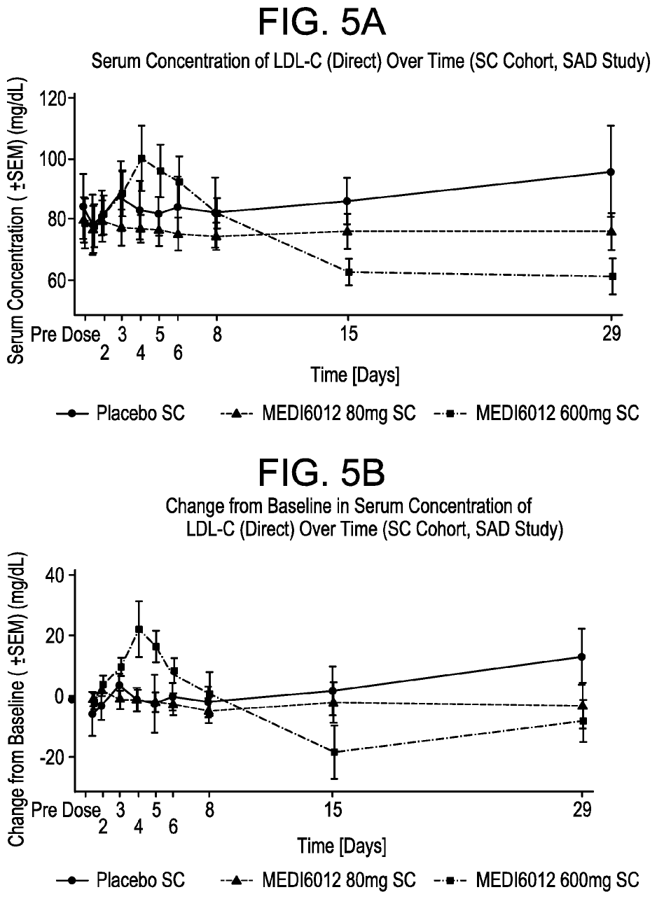

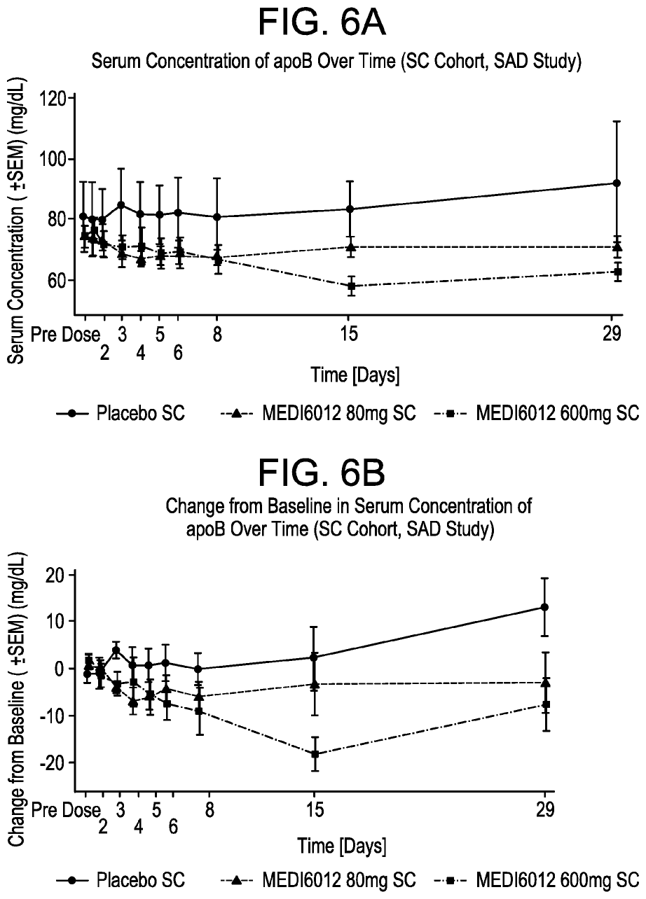

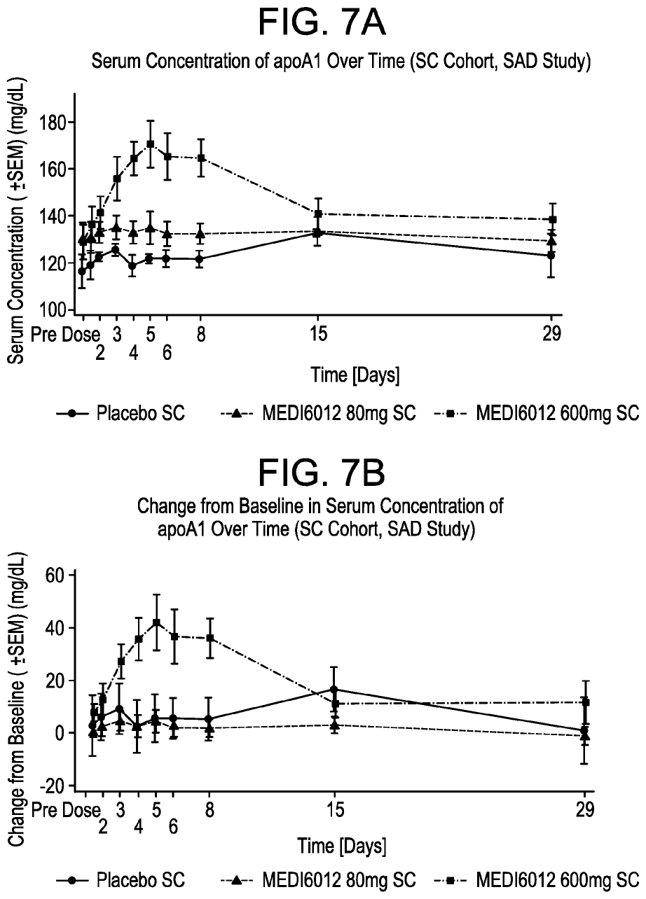

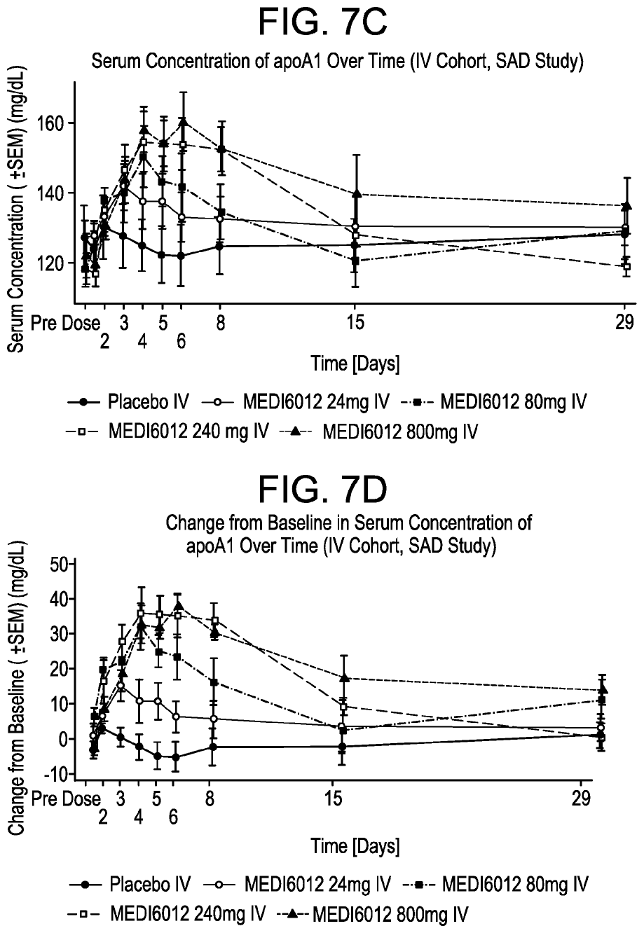

In subjects who received subcutaneous (SC) administration of MEDI6012 at doses of 80 and 600 mg, a dose-dependent increase of HDL-C was observed over time for both of the doses compared with placebo, based on serum concentration of HDL-C over time (FIG. 4A) and change from baseline in serum concentration of HDL-C over time (FIG. 4B). As shown in FIGS. 5A and 5B, no significant change was observed in LDL-C levels across all doses of MEDI6012 by SC administration, i.e., 80 and 600 mg. Similar to the results found for IV administration of MEDI6012, decreases in apoB were observed over time for both of the SC doses of MEDI6012 compared with placebo. (FIGS. 6A and 6B). The administration of MEDI6012 resulted in a statistically significant increase in serum concentrations of apoA1 over time in the 600 mg dose group that had received MEDI6012 by SC administration (FIGS. 7A and 7B), as well as across all of the IV doses (24 mg, 80 mg, 240 mg and 800 mg) of MEDI6012 administered to subjects over time (FIGS. 7C and 7D).

In subjects who received intravenous (IV) administration of MEDI6012 at doses of 24 mg, 80 mg, 240 mg and 800 mg, a dose-dependent increase of HDL-C was observed over time, especially during an approximately 8-12 day time period for each of the doses compared with placebo, based on serum concentration of HDL-C over time (FIG. 4C) and change from baseline in serum concentration of HDL-C over time (FIG. 4D). The increase in HDL-C was particularly pronounced during the first 2-5 and even 8 days following IV administration at the indicated doses of MEDI6012.

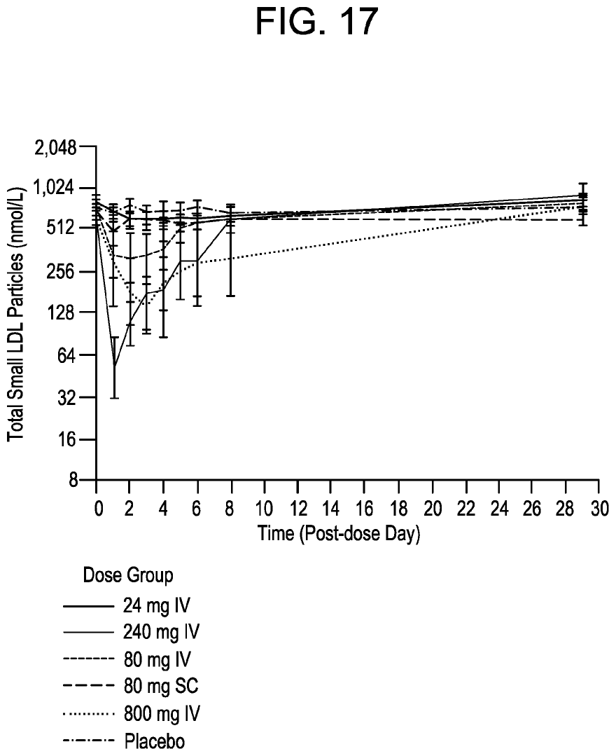

Further results showed that the method afforded anti-atherogenic effects based on decreases observed in small LDL particles (LDL-P), which are atherogenic agents which, due to their small size, can infiltrate blood vessel walls and damage the vessels. In particular, the results of the methods demonstrated that a decrease in small LDL-P was about 40-41% at a MEDI6012 dose of 80 mg and that a decrease in small LDL-P was about 80% at a MEDI6012 dose of 240 mg with no additional increase at a dose of 800 mg. This is shown by the results presented in FIG. 17. Therefore, doses of MEDI6012 in an amount of 80 mg-240 mg caused substantial decreases in detrimental LDL-P levels, thus providing additional therapeutic and cardio- and cardiovascular protective benefits afforded by the described methods.

In summary, the SAD study demonstrated that a single infusion of MEDI6012 (LCAT enzyme) in coronary heart disease (CHD) patients on background statin therapy caused dose dependent increases in HDL cholesterol (HDL-C), HDL cholesteryl ester (HDL-CE), and total CE; consistent with the mechanism of action of LCAT. In addition, a single dose of MEDI6012 caused dose-dependent increases in apolipoprotein A1 (ApoA1) that peaked at doses between 80 mg and 240 mg.

### Example 2—Clinical Trial Involving the Treatment of Subjects Having Stable Atherosclerotic Cardiovascular Disease (CVD) with Repeat Doses of MEDI6012 (rhLCAT)

### Overall Trial Design

A Phase 2a randomized, blinded, placebo-controlled study was designed to evaluate the safety, pharmacokinetics and pharmacodynamics of multiple (multiple ascending doses (MAD)) involving repeat weekly dosing of MEDI6012 in subjects with stable atherosclerotic cardiovascular disease. This dose escalation study was carried out to evaluate the safety, PK/PD and immunogenicity of repeat doses of MEDI6012 in adult subjects with stable atherosclerotic CVD. At least 32 subjects were randomized across approximately 10 study sites in the United States (USA) to evaluate 4 dose levels of MEDI6012 (40, 120, 300 mg) (in cohorts 1-3), and an IV push dosing regimen that included a loading (first) dose of 300 mg followed by a 150 mg maintenance (second) dose at 48 hours and a 100 mg maintenance (third) dose of MEDI6012 about a week (7 days) later (cohort 4, as described in Example 3, infra). The MEDI6012 investigational product was administered to subjects in Cohorts 1-3 weekly via intravenous (IV) infusion. Evaluations of the effects of MEDI6012 dosing in study subjects of cohorts 1-3 have been made as the ongoing study has progressed, as described herein. Cohort 4 of the MAD study is described in Example 3 infra.

For each cohort, 8 subjects were randomized in a 6:2 ratio to receive MEDI6012 or placebo. For subjects in cohorts 1-3, MEDI6012 investigational product was intravenously administered as a 1-hour IV infusion, and certain interim analyses of patient data were made.

For the ongoing MAD study, the subjects in cohorts 1-3 underwent a screening period of up to 28 days. For subjects requiring a washout of dyslipidemia medication or supplement, a 56 day screening period was allowed. Subjects were admitted to the study center the evening prior to randomization and first dose administration (Day −1) and prior to third dose administration and could, if desired, remain at the study center for 24-36 hours. For dose 2, subjects were observed as inpatients for at least 8 hours following dosing. For Cohort 4, outpatient arrangements may be provided through Day 4. Subjects were followed as outpatients through 56 days after the last dose of investigational product (Day 71 visit for Cohorts 1-3, Day 66 visit for Cohort 4). Subjects were encouraged to maintain a healthy lifestyle, including diet and exercise, during the study period.

### Target Subject Population and Investigational Product, Dosage and Mode of Administration

The target subject population for this study included adult men or women, aged 60 through 80 years, with a history of documented stable atherosclerotic CVD.

The MEDI6012 investigational drug product dosage and mode of administration for

Cohorts 1-3 were as follows:
Cohort 1: 40 mg MEDI6012 (n=6) or placebo (n=2) IV on Days 1, 8, and 15;
Cohort 2: 120 mg MEDI6012 (n=6) or placebo (n=2) IV on Days 1, 8, and 15; and
Cohort 3: 300 mg MEDI6012 (n=6) or placebo (n=2) IV on Days 1, 8, and 15.

Sample Size:

As noted supra, the at least 32 subjects enrolled in the ongoing study were in cohorts, each having 8 subjects randomized in a 6:2 ratio to receive MEDI6012 or placebo. The sample size for this multiple-ascending dose study was empirically determined to provide adequate safety, tolerability, and PK/PD data to achieve study objectives.

Statistical Analyses:

Safety analysis was based on the As-treated Population. Adverse event (AE) and serious adverse event collection began after the subject signed the informed consent document and lasted until the end of study visit. TEAEs and TESAEs were coded by the most updated version of the Medical Dictionary for Regulatory Activities (MedDRA), and the type incidence, severity, and relationship to investigational product was summarized. Specific AEs were counted once for each subject for calculating percentages. In addition, if the same AE occurred multiple times within a particular subject, the highest severity and level of relationship observed were reported. All TEAEs and TESAEs were summarized overall, as well as categorized by MedDRA system organ class and preferred term.

The PD parameters of primary interest are the baseline adjusted AUC from time 0 to 96 hours post dose 3 in HDL-C (AUC₀₋₉₆ hr Dose 3), HDL-CE, and CE. AUC was calculated using the trapezoidal rule. Statistical comparison among treatment groups with placebo group combined was conducted using analysis of covariance (ANCOVA) by adjusting baseline HDL-C, HDL-CE, and CE and treatment group. Other endpoints, including AUC₀₋₉₆ hr Dose 1, AUC₀₋₁₆₈ hr Dose 1, AUC₀₋₁₆₈ hr Dose 3 (AUC from time 0 on Day 1 through 168 hours after Dose 3), AUC₁₋₂₂ d, HDL-C, TC, FC, CE, HDL-CE, HDL-UC, non-HDL-C, non-HDL-CE, non-HDL-UC, LDL C (via direct measure by a standard laboratory test), apoA1, and apoB, were analyzed similarly to the primary PD endpoint.

Change and the percent change from baseline at each time point for each of the above lipids, lipoproteins, and apolipoproteins as well as VLDL-C, TG, preβ1-HDL, apoA1, apoA1l, apoCIII, and apoE were analyzed and compared using ANCOVA by adjusting baseline and treatment group with placebo group combined. For ANCOVA, if the data were not normally distributed, the analyses were conducted on rank-transformed data.

Descriptive statistics were provided by treatment group for maximum biomarker response (Rₘₐₓ) and time to reach maximum biomarker response ([R] Tₘₐₓ) for each of these as well.

ADA incidence rate and titer were tabulated for each treatment group assessed. Samples confirmed positive for ADA were tested and analyzed for nAb and summarized similarly. Non-compartmental analysis was performed for MEDI6012 treated subjects. Serum MEDI6012 mass and activity concentration-time profiles were summarized by dose cohort. The PK parameters to be reported included maximum plasma concentration of the drug (Cₘₐₓ), time of maximal concentration (Tₘₐₓ), AUC, accumulation ratio and terminal half-life (t_{1/2}). Descriptive statistics for PK parameters were provided.

Additional PK analyses were conducted as appropriate. If the data allowed, population PK analysis were performed but were not reported within the clinical study report (CSR).

Primary analysis: A primary analysis of the safety, immunogenicity, PK, and PD data was conducted after the last subject had completed or dropped out prior to the last scheduled visit (Day 71 for Cohorts 1-3) and were reported in the CSR.

### Primary, Secondary and Exploratory Objectives of the Study

The primary safety objective of the MAD study was to evaluate the safety of MEDI6012 following repeat dosing in subjects with stable atherosclerotic CVD over time to Day 71 for Cohorts 1-3, or to Day 66 for Cohort 4 (Example 3). The primary PD objective was to establish that repeat dosing with MEDI6012 resulted in a sustainable and reversible dose-dependent response for the PD HDL-C, HDL-CE, and CE, the levels of which were evaluated during the study.

The secondary objectives of the study were to establish the PK profile of MEDI6012 following repeat-dose administration; to evaluate the effect of MEDI6012 on a range of PD biomarkers following repeat dose administration; and to evaluate the immunogenicity potential of MEDI6012. An exploratory objective of the study was to explore biomarkers of high-density lipoprotein (HDL; and low-density lipoprotein (LDL) and very low-density lipoprotein (VLDL)) size, composition, and function.

### Study Endpoints

Safety and tolerability of MEDI6012 as measured by the incidence of treatment-emergent adverse events (TEAEs) and treatment-emergent serious adverse events (TESAEs) and clinically important changes in 12-lead electrocardiogram, vital signs, and clinical laboratory evaluations over time to Day 71 for cohorts 1-3:

### Primary PD Endpoint:

Baseline adjusted area under the concentration-time curve from time 0 to 96 hours post dose 3 (AUC_{0-96 hr}) for HDL-C, HDL-CE, and CE.

### Secondary Endpoints:

PK for MEDI6012 mass and activity. Serum concentration of other key lipids and lipoproteins: CE, HDL-CE, HDL-unesterified cholesterol, (HDL-UC), non-HDL-C, non-HDL-CE, non-HDL-UC, low density lipoprotein cholesterol (LDL-C), total cholesterol (TC), apolipoprotein B (apoB), triglycerides (TG), very low-density lipoprotein-cholesterol (VLDL-C), free cholesterol (FC), and apoA1, apoA1l, apoCIII, apolipoprotein E (apoE), preβ1-HDL; and anti-drug antibodies (ADA) and nAb development, with concomitant decreases in HDL-C.

### Exploratory Endpoint:

Measurement of lipoprotein particle size, number, function, and other assays assessing the effects of changes in lipid metabolism.

### Study Design Dose Rationale

Without wishing to be bound by any particular theory, the premise of the clinical development of MEDI6012 is that the administration of MEDI6012 (rhLCAT) in patients with atherosclerotic CVD will upregulate mobilization of cholesterol from tissues, including cholesterol from atherosclerotic plaques in coronary, cerebrovascular, and peripheral arteries, resulting in their stabilization and a consequent decreased risk for recurrent major adverse CV events. In addition, expected improvements in HDL function may result in the modulation of inflammation and improvements in endothelial function, effects that may also contribute to a reduction in major adverse CV events.

As indicated in Example 1, a single dose of MEDI6012 administered to subjects in amounts of 24, 80, 240, or 800 mg IV and at 80 or 600 mg SC showed an acceptable safety profile and dose-dependent increases in HDL-C, HDL-CE, and CE. MEDI6012 was therefore evaluated in the MAD study using a multiple ascending dose design to characterize the clinical PK and PD, as well as its safety and immunogenicity in a repeat-dose setting. The protocol is identified as a Phase 2a study, because the primary PD endpoint is statistically powered for evaluation in a target subject population and builds upon the data from the Phase 2a single ascending dose study (Example 1).

The Phase 2a, multiple-dose-escalation study was designed to provide PK/PD, safety, and immunogenicity data for repeat administration of MEDI6012 in a stable atherosclerotic CVD population. The subjects participating in this study had established atherosclerosis in at least one vascular bed (coronary, carotid, or peripheral arteries). In cohorts 1-3, subjects were given three IV doses on a weekly basis. In cohort 4, subjects were given a loading dose on Day 1 and maintenance doses on Days 3 and 10 via IV push, as described in Example 3, infra. It is expected that subjects may see transient changes in lipid/lipoprotein parameters based on the 3 dose drug regimen and duration of the study. Durable therapeutic benefit is envisioned for longer term dosing with MEDI6012, using the repeated dosing regimens as described herein. Subject risk was minimized through strict eligibility criteria to avoid enrollment of unstable or high-risk subjects and by close monitoring of adverse events (AEs), laboratory parameters, vital signs, and electrocardiograms (ECGs). In addition, PK, PD and immunogenicity were evaluated on an ongoing basis over the course of the study.

The primary hypothesis of the study is that repeat dosing with MEDI6012 exhibits an acceptable safety profile in subjects with stable atherosclerotic cardiovascular disease (CVD) and that repeat dosing with MEDI6012 results in a sustained and reversible dose-dependent response for high-density lipoprotein-cholesterol (HDL-C), cholesteryl ester (CE), and high-density lipoprotein-cholesteryl ester (HDL-CE) that allows once weekly or less frequent dosing of the drug. Secondary hypotheses related to the study are that (i) repeat dosing with MEDI6012 results in a pharmacokinetic (PK) profile (lecithin-cholesterol acyltransferase (LCAT) mass and LCAT activity) that allows once weekly or more frequent dosing; (ii) a regimen comprised of an initial loading dose of MEDI6012 followed by a dose at 48 hours and 1 week later results in a rapid rise in HDL-C and/or apoA1 compared with no loading dose, as well as maintenance of pharmacodynamic (PD) effect for 7 or more; (iii) repeat dosing with MEDI6012 results in dose-dependent responses for other key pharmacodynamic (PD) biomarkers in subjects with stable atherosclerotic CVD; and (iv) repeat dosing with MEDI6012 does not result in the development of neutralizing anti-drug antibodies (nAb) that cross-react with endogenous LCAT leading to decreased HDL-C.

### Primary, Secondary and Exploratory Objectives and Endpoints of the Study

The primary safety objective of the study involves evaluation of the safety of MEDI6012 following repeat dosing in subjects with stable atherosclerotic CVD over time to Day 71 for cohorts 1-3. The primary PD objective is to establish that repeat dosing with MEDI6012 results in a sustainable and reversible dose-dependent response for HDL-C, HDL-CE, and CE.

The primary safety endpoint takes into account the safety and tolerability of MEDI6012 as measured by the incidence of TEAEs and TESAEs and clinically important changes in 12-lead ECG, vital signs, and clinical laboratory evaluations over time to Day 71 for cohorts 1-3. The primary PD endpoint is baseline adjusted area under the concentration time curve from time 0 to 96 hours post dose 3 (AUC₀₋₉₆ hr) for HDL-C, HDL-CE, and CE.

Rationale for Primary Endpoint:

The primary PD endpoints of HDL-C, HDL-CE, and CE were analyzed as baseline-adjusted AUC_{0-96 hr} following the third dose of MEDI6012 in assessed subjects. Since rhLCAT esterifies free cholesterol in HDL, it is expected that the effectiveness of MEDI6012 correlates with changes in HDL-C, HDL-CE, and CE levels. This relationship has held true based on previous studies and the SAD study in stable CAD subjects receiving MEDI6012 (Example 1). This is also supported by a MEDI6012 cynomolgus monkey toxicology study (normal animals with intact endogenous rhLCAT and high levels of HDL-C) that showed a robust and transient increases in HDL-C following MEDI6012 infusion.

The secondary objectives are to establish the PK profile of MEDI6012 following repeat dose administration; to establish the PD effect of MEDI6012 following an initial loading dose followed by a dose at 48 hours and 1 week later (Cohort 4 only, Example 3); to evaluate the effect of MEDI6012 on a range of PD biomarkers following repeat weekly dose administration; and to evaluate the immunogenicity potential of MEDI6012.

The secondary endpoints involve assessments of PK for MEDI6012 mass and activity; serum concentration of other key lipids and lipoproteins: CE, HDL-CE, HDL-UC, non-HDL-C, non-HDL-CE, non-HDL-UC, LDL-C, TC, apolipoprotein B (apoB), triglycerides (TG), very low-density lipoprotein-cholesterol (VLDL-C), FC, and apoA1, apoA1l, apoCIII, apolipoprotein E (apoE), pre-beta1 high-density lipoprotein (preβ1-HDL; and ADA and nAb development, with concomitant decreases in HDL-C.

Rationale for PD and PK Endpoints:

Lipoproteins and lipid panel components were selected because movement of these markers, as a result of MEDI6012 dosing, provide supporting evidence of increased activity on the reverse cholesterol transport (RCT) system in the single ascending dose study. Total cholesterol (TC) represents the sum of unesterified and esterified cholesterol on all plasma lipoproteins. HDL-C represents the amount of cholesterol present in HDL particles, which can be further divided into HDL-UC and HDL-CE fractions. Through its enzymatic activity, MEDI6012 is expected to result in increases in HDL-C, the primary PD biomarker. TC and LDL-C further assess the effects of rhLCAT on RCT and were therefore identified as secondary PD endpoints.

Serum concentration of MEDI6012 (mass) was used to characterize MEDI6012 exposure. The PK was also used to develop dose-exposure-PD response relationships to help inform dose selection for future clinical studies. Serum LCAT activity provides an alternative measure to LCAT mass in establishing the relationship between PK and PD.

The exploratory objective of the study is to explore biomarkers of HDL (and low-density lipoprotein (LDL) and very low-density lipoprotein (VLDL)) size, composition, and function following MEDI6012 administration. The exploratory endpoint involves measurement of lipoprotein particle size, number, function, and other assays assessing the effects of changes in lipid metabolism. The rationale for exploratory PD endpoints (lipoprotein particle size and particle number) was the same as that for the SAD study described in Example 1.

### Treatment Regimen

For cohorts 1-3 of the MAD study, the enrollment of 32 subjects evaluated 3 dose levels of MEDI6012 via IV infusions (40, 120, and 300 mg) with a repeat-dose administration as presented in Table 5 below. PD and PK were analyzed as they became available during the arms of the study.

TABLE 5
MAD study treatment regimens for Cohorts 1-3

| | | Number of Doses | | |
|---|---|---|---|---|
| | | and Dosing | Number of | |
| Cohort | Dose | Frequency | Subjects | Randomization |
| 1 | 40 mg IV | 3 once weekly | 8 | 6 MEDI6012: 2 |
| | | doses | | Placebo |
| | | (Days 1, 8, 15) | | |
| 2 | 120 mg IV | 3 once weekly | 8 | 6 MEDI6012: 2 |
| | | doses | | Placebo |
| | | (Days 1, 8, 15) | | |
| 3 | 300 mg IV | 3 once weekly | 8 | 6 MEDI6012: 2 |
| | | doses | | Placebo |
| | | (Days 1, 8, 15) | | |

### Investigational Drug (MEDI6012) and Treatment Administration

The MEDI6012 investigational drug, placebo and IVBP solution for the MAD study are as described for the SAD study in Example 1.

The administration of MEDI6012 by infusion to cohorts 1-3 in the MAD study followed the same protocol as that used in the SAD study described in Example 1.

For treatment administration, the first day of MEDI6012 dosing is considered Day 1. On each day of dosing, following an overnight fast for a minimum of 6 hours, investigational product is administered to a subject as soon as is practicable after rising. The MEDI6012 investigational product is administered to a subject via IV infusion over a period of approximately 60 minutes (±5 minutes) for cohorts 1-3. During the study, subjects continue to take any other medications prescribed to them, such as statin therapy, at their regular prescribed dose(s), and any other medication(s) prescribed for their atherosclerotic CVD.

### Study Design and Dose Rationale for MAD Study Cohorts 1-3

Doses for Cohorts 1-3 in the MAD study were selected based on preliminary PK/PD analysis that integrated Cohort 1 to Cohort 3 PK/PD data from the single-ascending dose study of MEDI6012 (Example 1). A dose-dependent increase in biomarkers of LCAT activity including HDL-C, HDL-CE, and CE was observed for MEDI6012 following administration of single-ascending doses (Cohort 1 to Cohort 3, 24-240 mg IV).

Statistical evaluation, definition of the analysis population (As-treated population) and sample size and power calculations are all as described for the SAD study in Example 1, supra.

### Study Results

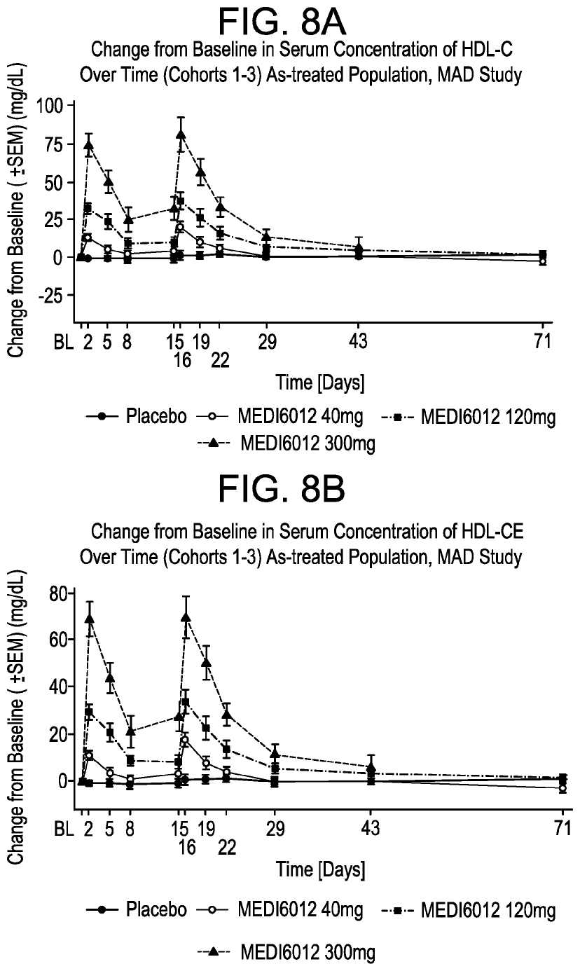

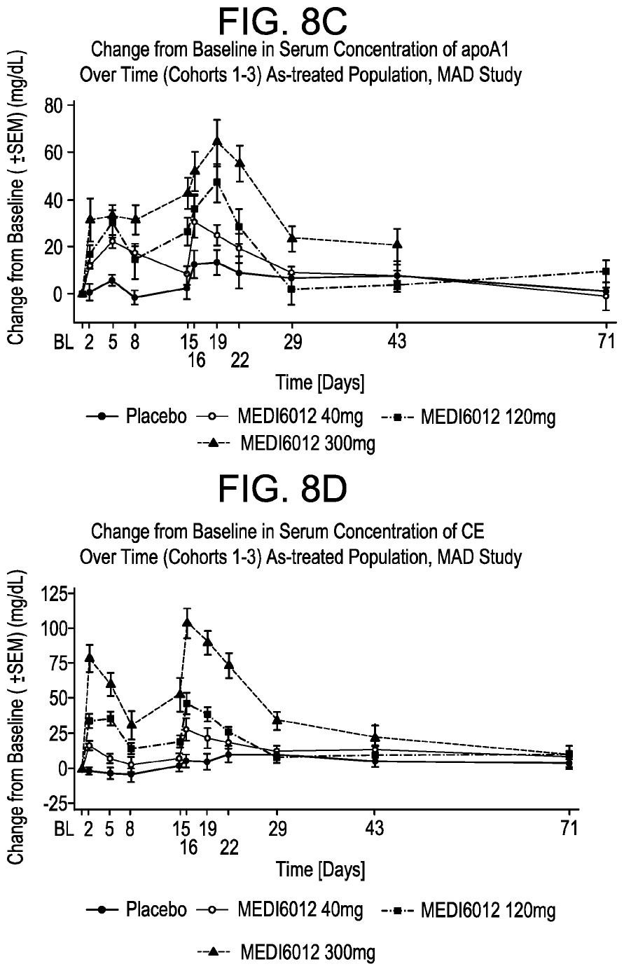

Analysis of the results from cohorts 1-3 of the ongoing MAD study showed that the administration of MEDI6012 to study subjects in these repeat dosing regimens, namely, a dose of 40 mg of MEDI6012 administered to the subjects by IV infusion on days 1, 8 and 15 (cohort 1); a dose of 120 mg of MEDI6012 administered to the subjects by IV infusion on days 1, 8 and 15 (cohort 2), and a dose of 300 mg of MEDI6012 administered to the subjects by IV infusion on days 1, 8 and 15 (cohort 3), led to dose-dependent increases in HDL-C, HDL-CE, apoA1 and CE that is consistent with the mechanism of action of the LCAT enzyme. (FIGS. 8A-8D).

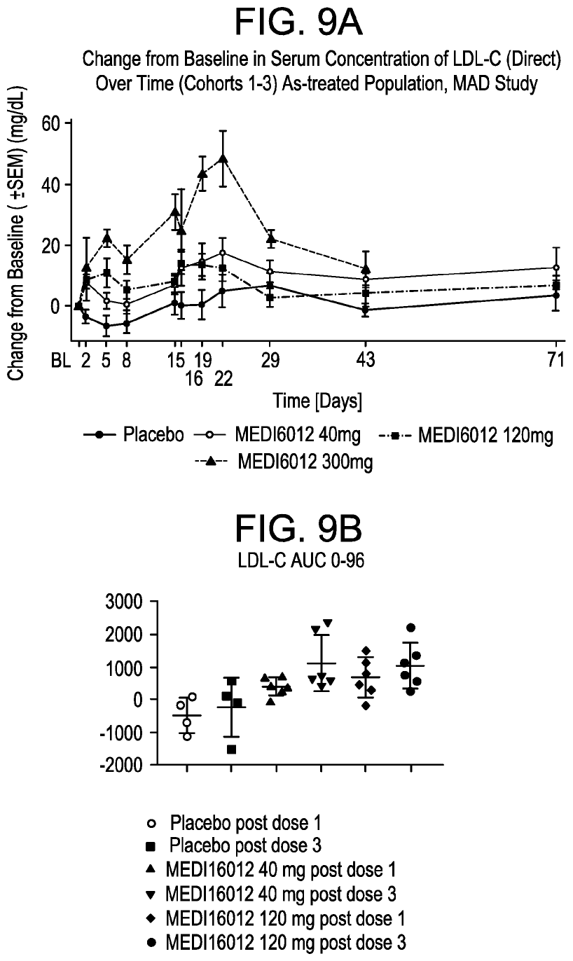

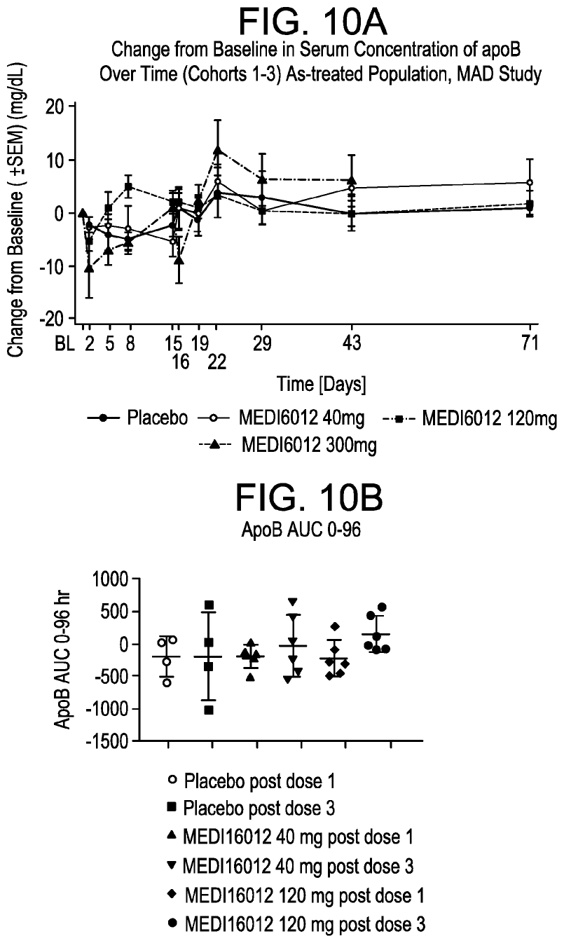

An increase in LDL levels was observed after the first dose of 120 mg and with the third dose of both 40 mg and 120 mg (FIGS. 9A and 9B); however, no increase in apoB was seen (FIGS. 10A and 10B). A number of reports have shown that apoB is a better predictor of risk of CHD than LDL-C in both men and women and that the number of atherogenic particles, such as apoB, is a more important indicator of risk than the amount of cholesterol (LDL-C) transported in these particles. (reviewed in Vaverkova, H., 2011, Clin Lipidology, 6(1):35-48; Sniderman, A D et al., 2003, Lancet, 361:777-780). Because all potentially atherogenic lipoprotein particles contain only one molecule of apoB and various amounts of cholesterol, apoB serves as a better marker of atherogenic lipoprotein particle numbers than LDL-C. Therefore, the little to no increase in apoB in subjects administered LCAT (MEDI6012) as demonstrated herein reflects a highly beneficial and protective treatment despite an observed increase in LDL-C.

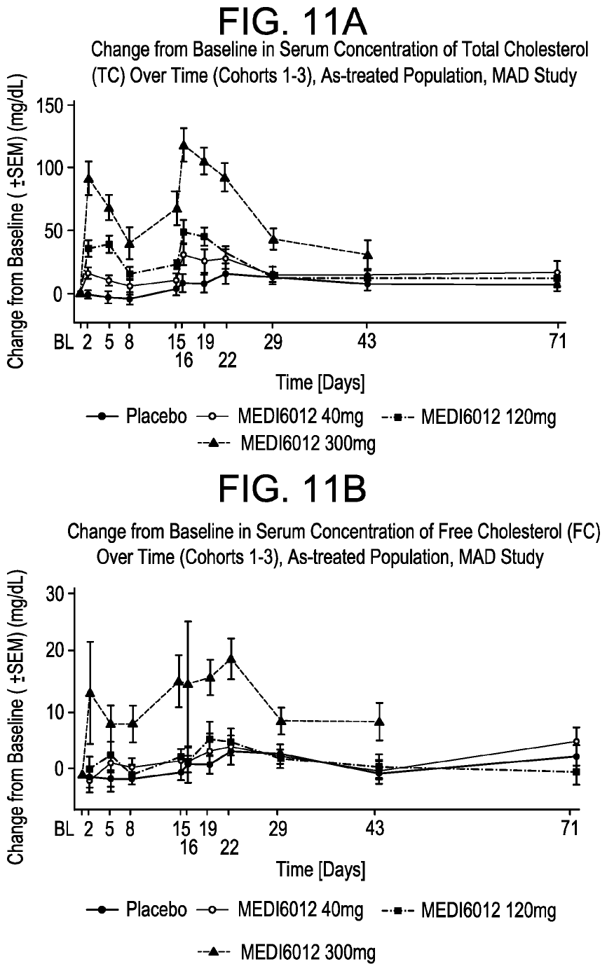

FIGS. 11A and 11B show that serum concentrations of total cholesterol (TC) and free cholesterol (FC) were elevated relative to placebo only at the highest dose of MEDI6012 (300 mg).

### Example 3

### Dose Selection Criteria for Cohort 4 of the MAD Study

PD observations from the single-ascending dose study of MEDI6012 as described in Example 1 and from the cohorts (cohorts 1-3) analyzed in the MAD study as described in Example 2 supra demonstrated that the rate of increase of HDL-C and apoA1 is dose dependent. For additional studies in subjects who have heart disease and/or cardiovascular disease, such as ACS and acute MI, maximizing the rate of increase of HDL-C and/or apoA1 following the first and second doses of MEDI6012, served as basis for the rationale that coupling the anti-atherosclerotic effects of enhanced reverse cholesterol transport with the cardioprotective effects of HDL-C and/or apoA1 (as also found following MEDI6012 dosing and administration in the cohorts described herein) would be expected to result in multiple benefits for CHD patients (as also supported by reports of Gordts et al, 2013, Gene Ther., 20(11):1053-61; Kalakech et al, 2014, PLoS One, 9(9):e107950; Marchesi et al, 2004, J. Pharmacol. Exp. Ther., 311(3):1023-31; Richart et al, 2015, Circulation, 132(Suppl 3):A17001-A; and Theilmeier et al, 2006, Circulation, 114(13):1403-9). Therefore, a cohort 4 was designed for addition to the MAD clinical study protocol in order to test IV administration of a loading dose of MEDI6012 by IV push, followed by 48 hour and then weekly maintenance doses of MEDI6012.

The primary and endpoint objectives of the cohort 4 study are those described for cohorts 1-3 in Example 2. Secondary objectives for the cohort 4 study involve establishing the PD effect of MEDI6012 after an initial loading dose, followed by doses at 48 hours and 1 week later.

The study involving cohort 4 is expected to effectively treat cardiac disease and/or cardiovascular disease in view of the results obtained to date from the SAD study (Example 1), in view of results obtained from subjects of cohorts 1-3 as described in Example 2, and in view of analytical modeling and simulation analysis and data performed to assess PD/PK and outcomes following the dosing regimen for cohort 4 as described herein.

Model and Simulation for Predicting the Effect of Intravenously Administered Doses of rhLCAT (MEDI6012) on Lipid and Protein Biomarkers in Treated Subjects

For the modeling and simulation analyses performed herein to determine effective doses and dosing regimens for MEDI6012 IV administration in the cohort 4 study subjects, a PK/PD model structure was employed. The PK/PD model structure was applied using MEDI6012 data in the SAD and MAD studies (Cohorts 1-3) for modeling and simulation to support dose selection for cohort 4.

The PK/PD model was based on the mathematical model developed for a reverse cholesterol transport (RCT) process involving PK/PD data and published data to select doses for a Phase 2a study involving the ACP501 rhLCAT (Bosch, R. et al., Poster entitled “A mechanism-based model is able to simultaneously explain the effect of rhLCAT and HDL mimetics on biomarkers of reverse cholesterol transport,” presented at the 2015 Population Approach Group in Europe (PAGE) Meeting, Hersonissos, Crete, Greece). The ACP501 mathematical model was developed to describe the effects of IV administration of rhLCAT and HDL mimetics (HDLm) on biomarkers of RCT in humans. The model described the time-dependent dynamics of lipid biomarkers within HDL particles by integrating literature and study data from two compounds with different mechanisms of action. The effects of HDL mimetics and rhLCAT preparations on RCT were integrated in the model, which described both the conformational change of the HDL particle from pre-β-HDL to αHDL, as well as the effect of the conformational change on the efflux of cholesterol.

Model Methods

For the modeling, MEDI6012, HDL-C, CE and apoA-I data were available from clinical and published studies. Similar to the model of R. Bosch et al., it was assumed that Total HDL-C=HDL-FC+HDL-CE and Total CE=HDL-CE+apoB-CE. PK models were developed that were highly similar to those for ACP501 and HDL mimetics, and the estimated PK parameters were fixed in the combined model. A literature study was performed to identify important pathways and reactions related to LCAT enzyme activity and function, and to obtain system specific parameter values. A PD model was developed for MEDI6012, and the model was updated to describe the effect of apoA1 on LCAT activity. Finally, the model was fitted simultaneously to the PD data after IV administration of MEDI6012 and apoA1. The model was evaluated and externally qualified using two independent clinical studies of HDL mimetics.

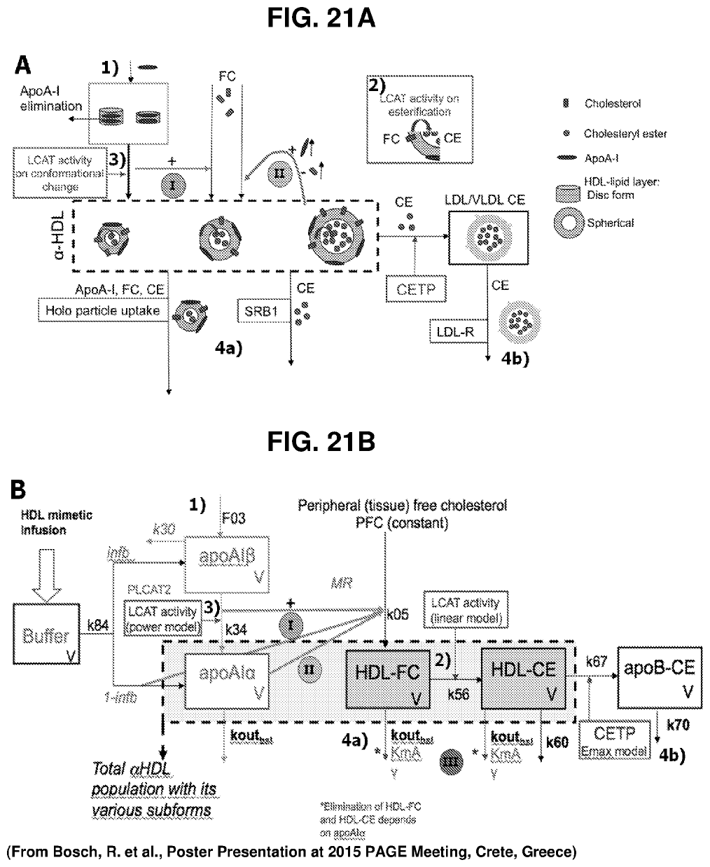

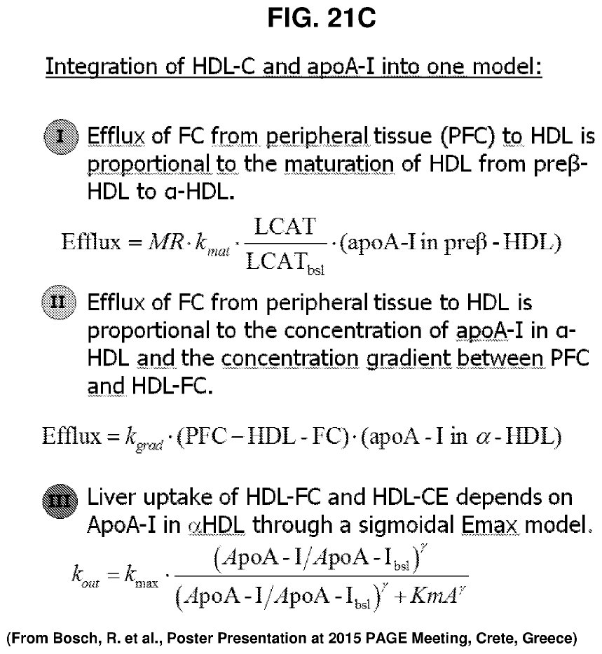

Several assumptions were made in connection with the modeling, as reported by Bosch, R. et al. in the 2015 PAGE Meeting poster, noted supra and as shown in FIGS. 21A-21C:

The input of apoA1 (in pre-(3 form) reflects its synthesis and is assumed constant. Recycling of apoA1 lead by remodelling of HDL particles after elimination was not considered. In the model, rate limiting steps involved in LCAT activity are considered during high doses of rhLCAT infusion.

Free cholesterol in tissue is assumed to be in abundance as compared to free cholesterol in plasma and therefore the free cholesterol concentration in tissue is fixed to a constant value.

It is assumed that the efflux of free cholesterol can be described by two processes. One process is dependent on the maturation of HDL; the other process is dependent on the concentration of apoA1 in α-HDL and the concentration of HDL-FC as compared to the (constant) concentration of free cholesterol in tissue.

The rates of elimination for HDL-CE, HDL-FC and apoB-CE are fixed to literature values. (e.g., Ouguerram K et al., 2002, A new labeling approach using stable isotopes to study in vivo plasma cholesterol metabolism in humans, Metabolism, 51:5-11).

The elimination of HDL-C(HDL-CE and HDL-FC) is assumed to increase with an increase from baseline in total apoA1.

It is assumed that that the transport of CE from HDL to apoB (LDL/VLDL) is dependent of the HDL-CE concentration and can be described by an Emax model.

LDL/VLDL were not considered separately, but were considered in conjunction with apoB.

The results of this modeling showed that although stimulation of RCT by HDL mimetics and rhLCAT were related to different mechanisms of action, the eight compartment mechanistic model was able to adequately describe both the observed plasma rhLCAT concentrations and the time-course of relevant biomarkers, including the fraction of esterified and unesterified cholesterol within HDL particles. Both internal and external model validation using VPC showed adequate model fit and good predictive performance HDLc AUC showed high correlation with the amount of cholesterol movement from the peripheral tissue and were useful for comparing the effects of HDL mimetics with rhLCAT on RCT.

### Doses, Predicted Outcomes and Results for Cohort 4 of the MAD Study

Treatment Regimen

The investigational drug product (rhLCAT or MEDI6012) dosage and mode of administration for Cohort 4 in the MAD study is as follows: 300 mg of rhLCAT or MEDI6012 is administered to subjects on Day 1 (loading dose); a second dose of 150 mg of rhLCAT or MEDI6012 is administered to subjects at 48 hours (±8 hours) (maintenance dose on Day 3); and a 100 mg dose of rhLCAT or MEDI6012 is administered to subjects 1 week following the second dose (maintenance dose on Day 10), all administered by IV push, as presented in Table 6 below. As noted supra, an IV push refers to the intravenous administration of rhLCAT or MEDI6012 (active drug or medication) which is typically manually delivered to a subject over a relatively short time period, for example and without limitation, over a time period of about 1-5 minutes, or over a time period of about 1-3 minutes. An IV push is typically administered to a subject via a syringe. An IV push may be delivered through a syringe into a short or long IV line into a vein or vessel of a subject.

TABLE 6
Cohort 4 MAD study treatment regimen

| | | Number of Doses | | |
|---|---|---|---|---|
| | | and Dosing | Number of | |
| Cohort | Dose | Frequency | Subjects | Randomization |
| 4 | 300 mg IV push (Day 1) | 3 doses | 8 | 6 MEDI6012: 2 Placebo |
| | 150 mg IV push (Day 3) | (Days 1, 3, 10) | | |
| | 100 mg IV push (Day 10) | | | |

The MEDI6012 investigational product is administered to a subject by IV push over approximately 1-3 minutes, inclusive of flush, for cohort 4. More specifically, for administration or delivery of MEDI6012 to a subject of cohort 4 by IV push in the MAD study, each IV push dose is administered or delivered as reconstituted MEDI6012 or placebo with a syringe and an IV administration set. IVBP is not used for preparation of doses for cohort 4. No incompatibilities have been observed with MEDI6012 in syringes (polycarbonate/polypropylene) and IV administration lines (PE/PVC and PVC DEHP-free). IV administration lines must contain either 0.22 or 0.2 nm in-line PES filter. Lines containing cellulose-based filters should not be used with MEDI6012, as these have not been tested. Dose 1 (300 mg) is administered as 3 separate injections. Each injection is administered over 30 seconds and each injection is followed by a 10 mL normal saline flush. Dose 2 (150 mg) is administered as 2 separate injections. Each injection is administered over 30 seconds, and each injection is followed by a 10 mL normal saline flush. Dose 3 (100 mg) is administered as a single injection over 30 seconds and followed by a 10 mL normal saline flush. Because MEDI6012 and placebo do not contain preservatives, any unused portion must be discarded. The total in-use storage time from needle puncture of the first investigational product vial(s) to start of IV push administration should not exceed 4 hours at room temperature. If storage time exceeds these limits, a new dose must be prepared from a new vial(s).

As described above, the loading dose and maintenance doses for cohort 4 were selected based on PK/PD analysis that integrated PK/PD data from the SAD study of MEDI6012 (Example 1) and PD data from the MAD study (Example 2). Simulations utilizing the RCT PK/PD model were performed based on the estimated PK/PD parameters to select doses for cohort 4 in the MAD study that characterized MEDI6012 PK and the range of PD effects when MEDI6012 was administered with loading and maintenance doses administered via IV push. The PD effect of increasing loading doses of MEDI6012 (160, 200, 240, 280, and 320 mg) administered by IV bolus over 1 minute were simulated followed by weekly maintenance doses of 80, 100, 120, and 160 mg. From these simulations, it was noted that HDL-C increased by over 50% over the first 90 minutes when higher doses of MEDI6012 was administered.

Based on the above-described PK/PD modeling and simulation of R. Bosch et al., PD/PK modeling and simulation conducted for MEDI6012 and Cohort 4 dosing included a 300 mg loading dose on Day 1; a 150 mg maintenance dose on Day 3; and a 100 mg maintenance dose on Day 10. A dosing regimen with a loading dose followed by maintenance doses on Day 3 and 10, respectively, was considered as the optimal dosing regimen that sustained elevations of HDL-C for 1 week. Day 3 was chosen, because most acute MI patients are hospitalized for >48 hours and the half-life of MEDI6012 is approximately 48 hours. The goal of the 48 hour dose was to prolong the elevation of apoA1 in the acute/subacute MI setting over the first 1-2 weeks. This regimen resulted in baseline adjusted HDL-C levels >30 mg/dL for greater than 1 week. The first week following acute MI in a patient is critical with respect to cardioprotective and vasculoprotective aspects of therapy. In addition, this regimen results in early apoA1 levels near at the peak seen with larger doses (up to 800 mg IV) used in the single-ascending dose study of MEDI6012 and therefore maintains apoA1 levels for >1 week. The 100 mg maintenance dose on Day 10 was selected because it maintains elevations in HDL-C, apoA1 and/or cholesteryl ester in the system without accumulation of cholesteryl ester and is being tested to determine if this is an appropriate maintenance dose for longer term dosing of MEDI6012 for future studies. It is apparent that the rise in LDL is secondary to the accumulation of CE in LDL particles.

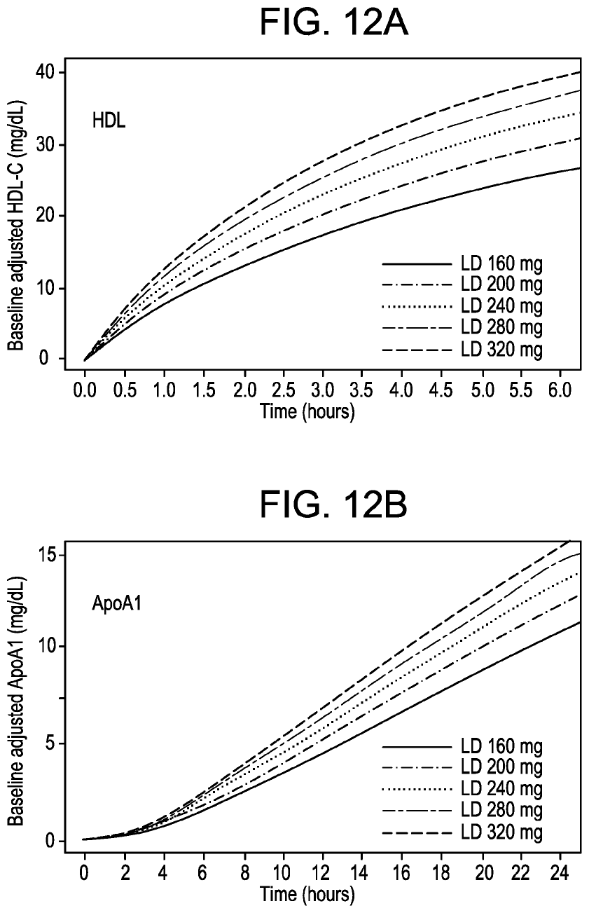

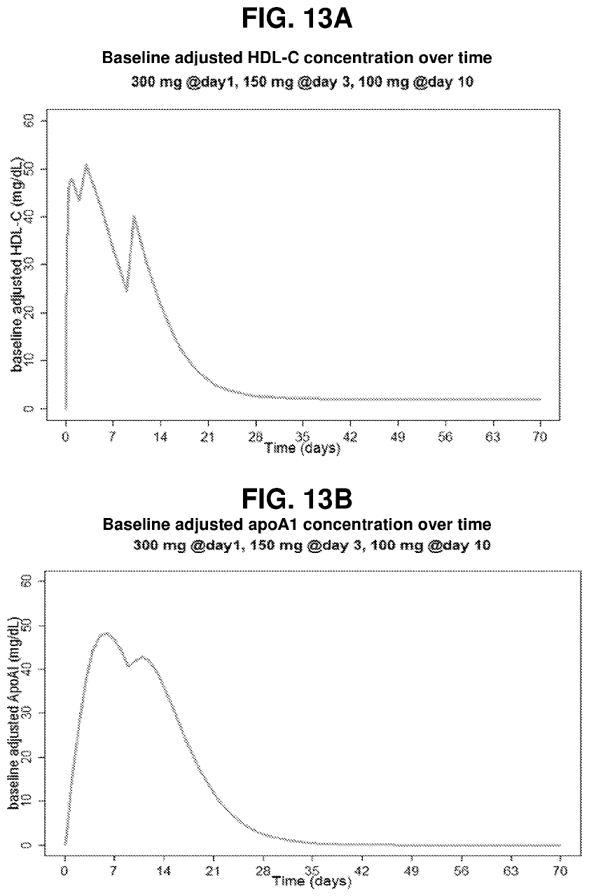

Specific selection criteria were associated with the modeling and simulation analyses to arrive at a dosing regimen of MEDI6012 for administration to the subjects in cohort 4 which had expected successful results and outcomes for cardioprotection, anti-atherogenic effects and minimal to no unwanted effects. Several different cardio-protective criteria were considered in the modeling and simulation analyses and assessments for determining the doses to be used for the subjects of cohort 4. One cardioprotective criterion for the modeling analysis included: rapid increase in HDL and apoA1 with the first dose (modeling data shown in FIGS. 12A and 12B, respectively). For HDL-C, a loading dose of 300 mg of MEDI6012 achieved approximately 39 mg/dL in 6 hours (FIG. 12A). For apoA1, a loading dose of 300 mg of MEDI6012 achieved 15 mg/dL over 24 hours (FIG. 12B). A second cardioprotective criterion for the modeling analysis included: maintaining HDL-C and apoA1 levels over a 2 week period (modeling data shown in FIGS. 13A and 13B, respectively). Based on the modeling data, it was determined that a loading dose of 300 mg of MEDI6012, followed by a 150 mg dose of MEDI6012 at 48 hours after the loading dose, followed by a 100 mg dose of MEDI6012 on Day 10 maintained HDL-C and apoA1 levels for 2 weeks, with apoA1 maintained at near maximal levels for 2 weeks. (FIGS. 13A and 13B).

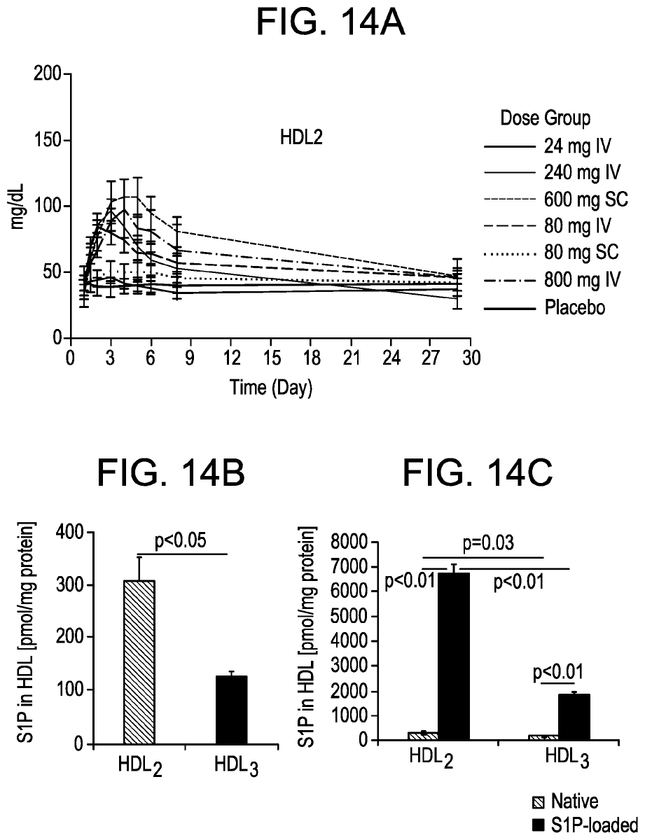

In addition, maintaining HDL-C and/or apoA1 levels high during a 2 week period after MEDI6012 dosing allows patients with MI to convert from an acute to a subacute stage of the disease and allows fibrosis repair in heart tissue, resulting in the proliferation of cardiomyocytes and the replacement of dead cardiomyocytes. A third cardioprotective criterion for the modeling analysis included: an increase in the HDL2 (HDL2-chol) subfraction of HDL, which contributes to cardioprotective and atherogenic protective effects at higher levels (compared with levels of the smaller density HDL3-chol subfraction). FIG. 14A shows increases in HDL2 at the different doses of MEDI6012 administered IV or SC. At a 240 mg dose of MEDI6012, HDL2 was predicted to increase by approximately 55 mg/dL. FIG. 14B shows that HDL2 is the subspecies of HDL that carries and accepts more sphingosine-1-phosphate (S1P) compared to HDL-3 as reported by Sattler, K. et al. (2015, J. Am. Coll. Cardiol., 66:1470-1485).

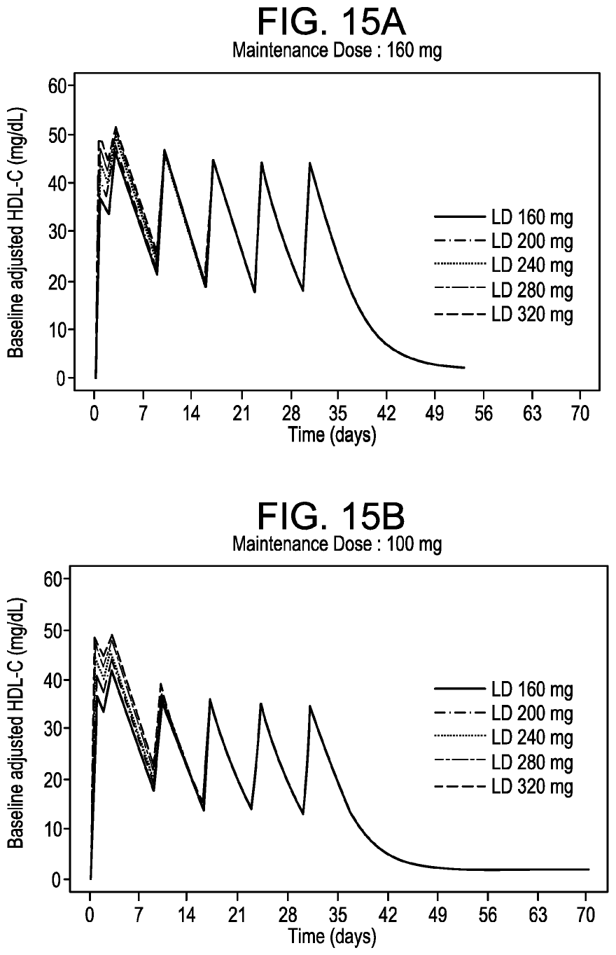

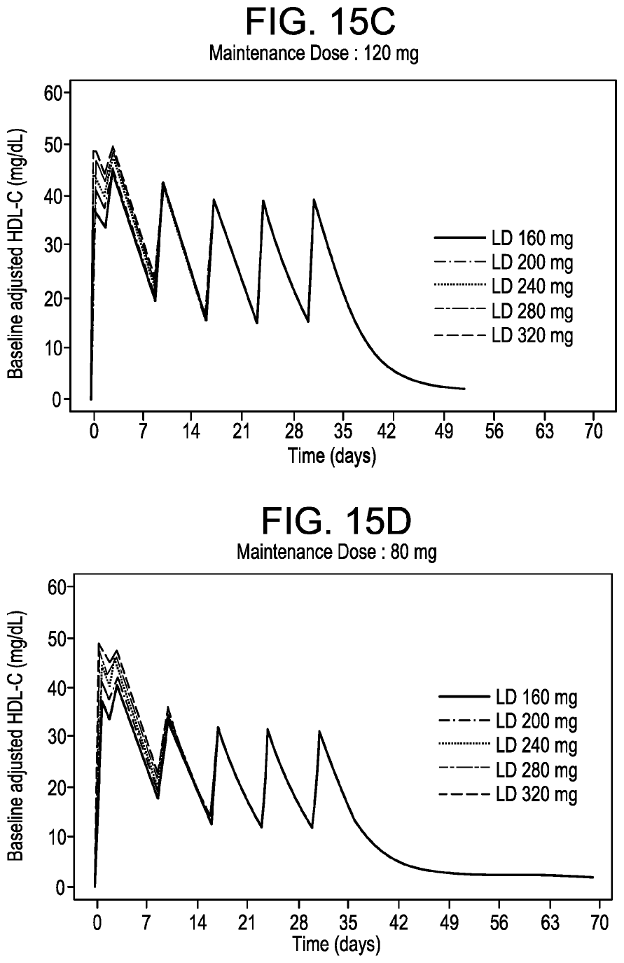

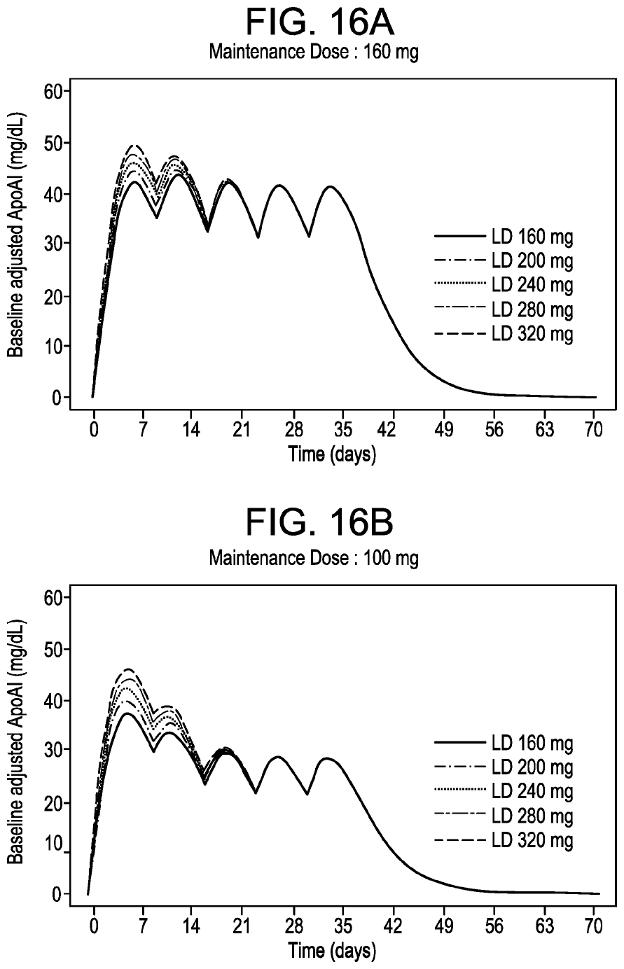

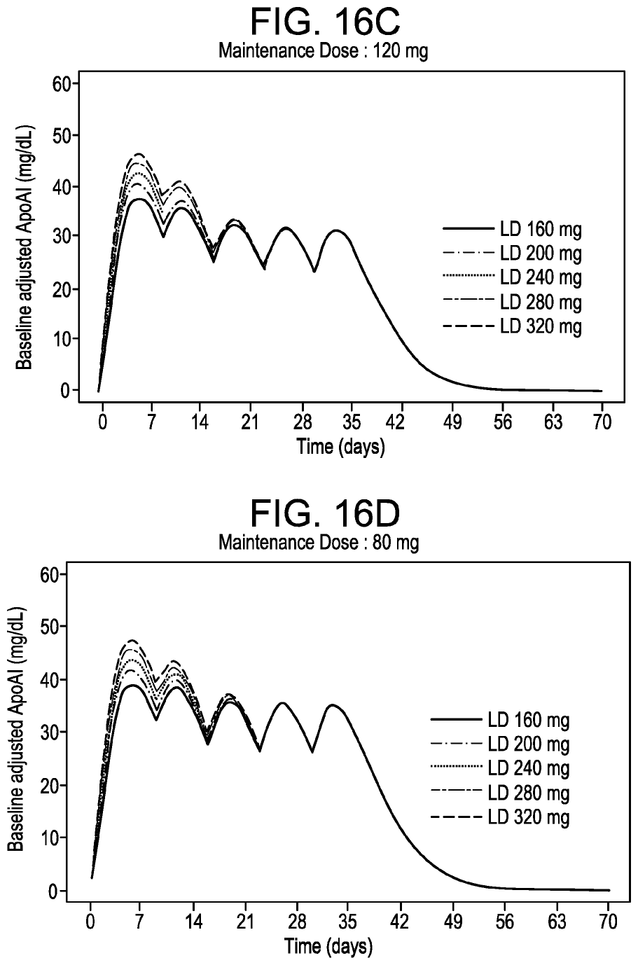

Several different athero-protective (anti-atherogenic) criteria were considered in the modeling and simulation analyses and assessments for determining doses, in particular, maintenance doses, to be used for the subjects of cohort 4. One anti-atherogenic criterion for the modeling analysis included: achieving HDL-C levels of greater than 60 mg/dL (baseline (BL)=35) in serum/plasma. Modeling predicted that in achieving HDL-C levels of greater than 60 mg/dL, the loading dose of MEDI6012 did not appreciably affect the steady state levels of HDL-C and a maintenance dose of approximately 100 mg of MEDI6012 was needed. This is shown by the results presented in FIGS. 15A-15D. Results from the study have shown that HDL-C levels above about 60 mg/dL, such as 65-80 mg/dL do not provide significantly more cardioprotective or atheroprotective effects for subjects than a level of 60 mg/dL. Another anti-atherogenic criterion for the modeling analysis included: maintaining apoA1 levels at steady state during maintenance dosing. Modeling predicted that all doses of MEDI6012 (i.e., 80 mg, 100 mg, 120 mg and 160 mg) achieved steady state levels of apoA1. This is shown by the results presented in FIGS. 16A-16D.

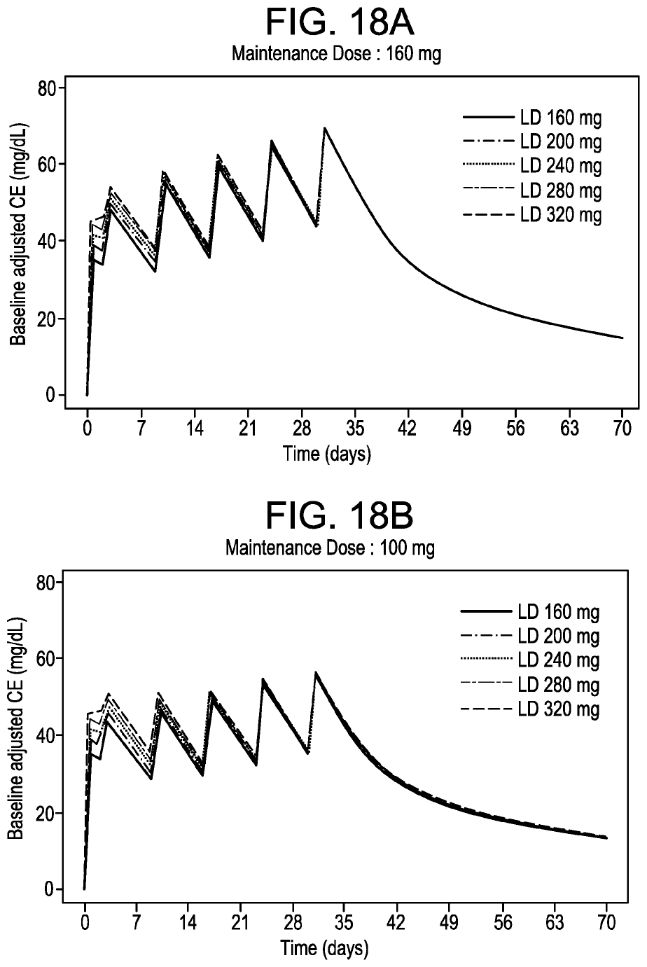

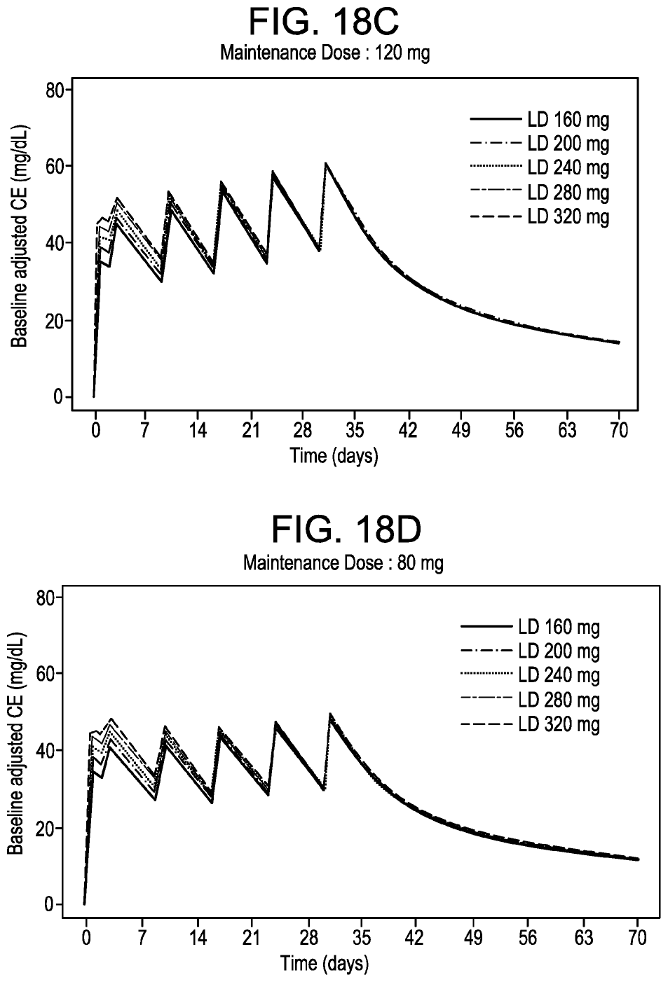

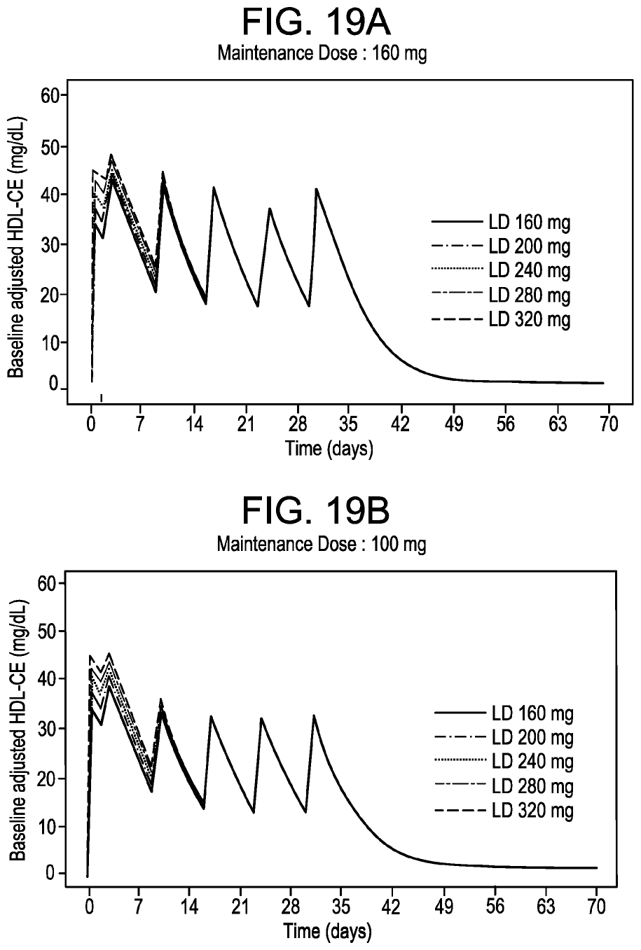

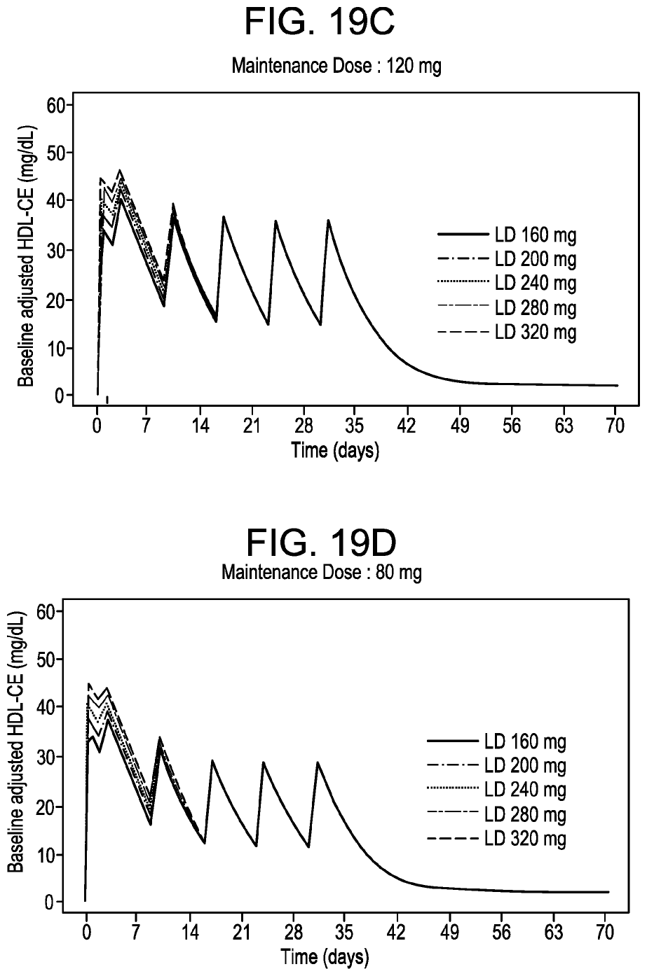

Also considered in the modeling analysis was the unwanted effect of cholesteryl ester accumulation in LDL with various maintenance doses of MEDI6012. The modeling and simulation predicted that a dose of MEDI6012 between 80-100 mg prevented too great an accumulation of CE. (FIGS. 18A-18D). Based on the modeling, little change was seen in HDL-CE levels at the various maintenance doses, indicating that cholesteryl ester accumulation was not in HDL. (FIGS. 19A-19D). While LDL data were not included in the modeling analysis per se, the modeling analysis was informed by observation of the results obtained from the SAD study which revealed increases in LDL levels at MEDI6012 doses ≥240 mg, as well as results from the MAD study, which revealed that a single 120 mg dose of MEDI6012 increased LDL levels and multiple doses of 40 mg or 120 mg increased LDL-C, but these doses did not cause increases in apoB. At maintenance doses of 120 mg of MEDI6012, LDL levels appeared to rise and CE accumulated with multiple dosing; however, there was minimal accumulation at 100 mg and minimal loss of CE at 80 mg. Thus, based on the HDL-CE and the observed LDL-C profiles, it was determined that CE was accumulating in LDL, rather than in HDL, at high maintenance doses, which provided an acceptable maintenance dose, especially for longer term dosing, for use in cohort 4 subjects so that they did not accumulate CE in LDL, for example, without limitation, a dose of ≤120 mg.

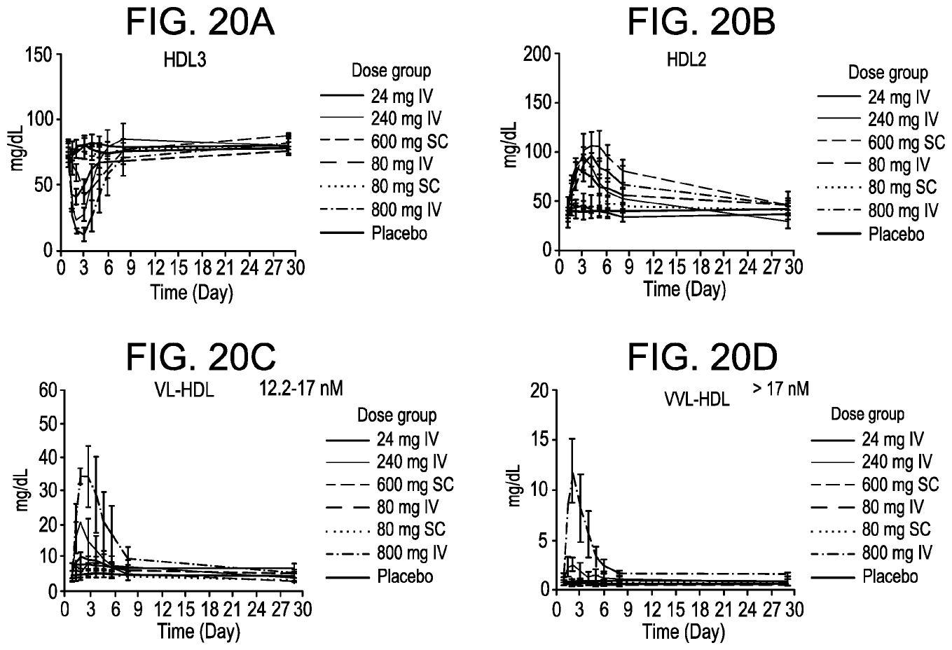

Another unwanted effect considered in the clinical studies was the situation in which there were no very very low density (VVL) HDL particles (>17 nM) and few very low density (VL)-HDL particles (12.2-17 nM). As HDL particles increase in size, they decrease in function. The LCAT enzyme, i.e., MEDI6012, plays a role in the conversion of the HDL3 particle subfraction of HDL to HDL2 particles, which are more cardioprotective and atheroprotective. (FIG. 20). The modeling analysis indicated that a 240 mg dose of MEDI6012 resulted in a 2 mg/dL increase in VVL-HDL and a 17 mg/dL increase in VL-HDL. An 80 mg dose of MEDI6012 resulted in no increase in VVL-HDL, and a 2 mg/dL increase in VL-HDL. The modeling studies also provided information that allowed a determination of those doses of MEDI6012 that would be suitable to avoid significant conversion of VL-HDL particles to VVL-HDL particles (FIG. 20).

In summary, based on rigorous modeling and simulation data and the accuracy expected from these data, and based on the observed data from clinical studies (as well as preclinical studies), the proposed dose regimens following three doses of MEDI6012 are expected to be well tolerated, and the collected PK/PD data are appropriate to fulfill the objectives of the study. The selection criteria used in the modeling and simulation analyses provided for the expected increases in HDL-C, apoA1, CE and other PD markers that would be efficacious in treating a subject's cardiac or cardiovascular diseases and/or symptoms thereof, without detrimental adverse effects. The selection criteria further allowed for the determination of a treatment regimen that was expected to provide therapeutic efficacy associated with the mechanism of action of the LCAT enzyme. The follow-up duration of 4 weeks after dosing is deemed appropriate to evaluate the reversibility of potential safety findings and to characterize the potential immunogenicity of MEDI6012 when the serum concentration (PK mass) has completely cleared and PD biomarkers return to baseline values. An additional duration of 4 weeks beyond the initial 4 weeks of follow-up is appropriate in ADA positive subjects to ensure there is not a decrease in HDL-C as a result of an ADA/nAb.

In particular, based on the modeling and simulation results described above, the effectiveness and achievement of certain loading and maintenance doses of MEDI6012 could predict the achievement of a successful outcome of the multiple dose study involving cohort 4, and thus validate the correlation between the expected treatment outcomes and the likelihood that the dosing and dosing regimens provide the predicted and expected results. Based on the modeling analyses as well as observed data, a 300 mg loading dose (LD) of MEDI6012, followed by 150 mg and/or 100 mg maintenance doses (MD) were predicted to achieve the following as cardioprotective criteria: (i) a rapid increase in HDL and/or apoA1 with the loading dose (a 300 mg LD of MEDI6012 achieves an HDL-C level of 39 mg/dL in 6 hours and an apoA1 level of 15 mg/dL in 24 hours); (ii) maintenance of HDL-C and apoA1 levels over a 2 week period (a 300 mg loading dose of MEDI6012 followed by a 150 mg dose at 48 hours, and followed by a 100 mg dose at Day 10 maintains protective levels of both HDL-C and apoA1 levels for 2 weeks, with apoA1 levels maintained at maximal levels for 2 weeks); and (iii) increased HDL2 levels (a 100 mg dose of MEDI6012 increases HDL2 levels by ˜55 mg/dL).

A 300 mg loading dose (LD) of MEDI6012, followed by 150 mg and/or 100 mg maintenance doses (MD), were also predicted to achieve the following as anti-atherogenic criteria: (i) achievement of HDL-C levels of >60 mg/dL (BL=35), (a 100 mg maintenance dose maintains HDL-C at a level of 60-70 mg/dL, assuming baseline (BL) levels of 35 mg/dL; (ii) maintenance of steady state apoA1 level); decrease in small LDL particles (a loading dose of 300 mg of MEDI6012 decreases small LDL-P by 80% and maintenance doses decrease LDL-P by 40-50% or higher); (iii) global efflux of cholesterol and efflux through the ATP-binding cassette transporter (ABCA1), also known as the cholesterol efflux regulatory protein (CERP), is expected to increase with a loading dose of MEDI6012 in an amount of 300 mg.

A 300 mg loading dose (LD) of MEDI6012, followed by 150 mg and/or 100 mg maintenance doses (MD) were further predicted to protect from unwanted effects following dosing. The modeling predicted no expected increase in apoB; no increase in LDL-C or cholesteryl ester accumulation in LDL (LDL-CE), (minimal to no increase in LDL or LDL-CE expected with maintenance doses of MEDI6012); and no VVL HDL and little VL-HDL increase (the loading dose produces an increase in VL-HDL and a minimal increase in VVL-HDL; a maintenance dose of 100 mg of MEDI6012 results in an increase of approximately 2 mg/dL in VL-HDL and no increase in VVL-HDL). The modeling data and results described supra serve as reliable predictors that forecast with accuracy and confidence, as well as validate, the outcomes of the actual treatment methods designed to employ the doses and dosing regimens of the MEDI6012 active ingredient, as detailed herein.

### Change in Treatment Regimen

As described above, Cohort 4 of the MAD study was designed so that 6 test subjects would receive rhLCAT or MEDI6012 and the 2 remaining subjects would receive the placebo dose. However, there was a randomization problem and 2 of the subjects had to be randomized manually. This led to 7 subjects being randomized to receive rhLCAT or MEDI6012 and only 1 subject randomized to receive the placebo. Therefore, the actual study treatment regimen is as presented below in Table 6a.

TABLE 6a
Actual Cohort 4 MAD study treatment regimen

| | | Number of Doses | | |
|---|---|---|---|---|
| | | and Dosing | Number of | |
| Cohort | Dose | Frequency | Subjects | Randomization |
| 4 | 300 mg IV push (Day 1) | 3 doses | 8 | 7 MEDI6012: 1 Placebo |
| | 150 mg IV push (Day 3) | (Days 1, 3, 10) | | |
| | 100 mg IV push (Day 10) | | | |

Additionally, the placebo subject, instead of receiving a placebo dose on Day 10, was administered a 100 mg dose of MEDI6012, and one of the test subjects, randomized to receive a 100 mg dose of MEDI6012 on Day 10, actually received a placebo dose in place of MEDI6012. Table 6b below shows the PK data from the placebo subject (Subject 20030810018) and the test subject randomized to receive MEDI6012 (Subject 20030810020). This assay is specific to MEDI6012. Therefore, a placebo subject who is not dosed with MEDI6012 should not have anything other than BLQ<2500 (Below Limit of Quantification).

As Table 6b demonstrates, after the IV push on Day 10 and the two subsequent sampling points, the placebo subject exhibits levels of MEDI6012 in their plasma. Additionally, Table 6b demonstrates that on Day 10, the test subject randomized to receive MEDI6012 is administered the placebo dose instead, as no MEDI6012 is detected in their plasma. Furthermore, it is believed that the placebo subject stopped taking their statin medication from Day 10 onwards, hence levels of LDL-C and ApoB begin to rise after Day 10.

TABLE 6b
PK data from placebo and test subjects

| | | Placebo subject | MEDI6012 subject |
|---|---|---|---|
| | Time | (Subject | (Subject |
| | Standard | 20030810018) | 20030810020) |
| Day 1 | Predose | BLQ < 2500 | BLQ < 2500 |
| (300 mg | 30 min post dose | BLQ < 2500 | No sample |
| MEDI6012 | End of IV push | BLQ < 2500 | No sample |
| or placebo) | 90 min post dose | BLQ < 2500 | No sample |
| | 4 hrs post dose | BLQ < 2500 | 49000 |
| | 12 hrs post dose | BLQ < 2500 | 38100 |
| | 24 hrs post dose | BLQ < 2500 | 27200 |
| Day 3 | Predose | BLQ < 2500 | 16900 |
| (150 mg | End of IV push | BLQ < 2500 | No sample |
| MEDI6012 | 6 hrs post dose | BLQ < 2500 | 39600 |
| or placebo) | 48 hrs post dose | BLQ < 2500 | 14800 |
| Day 10 | Predose | BLQ < 2500 | BLQ < 2500 |
| (100 mg | End of IV push | 8820 | BLQ < 2500 |
| MEDI6012 | 12 hrs post dose | 11000 | BLQ < 2500 |
| or placebo) | 24 hrs post dose | 10700 | BLQ < 2500 |
| | 96 hrs post dose | No sample | No sample |
| | 168 hrs post dose | BLQ < 2500 | BLQ < 2500 |
| | Day 24 | BLQ < 2500 | BLQ < 2500 |
| | Day 38 | BLQ < 2500 | BLQ < 2500 |
| | Day 66 | BLQ < 2500 | BLQ < 2500 |

### Study Results

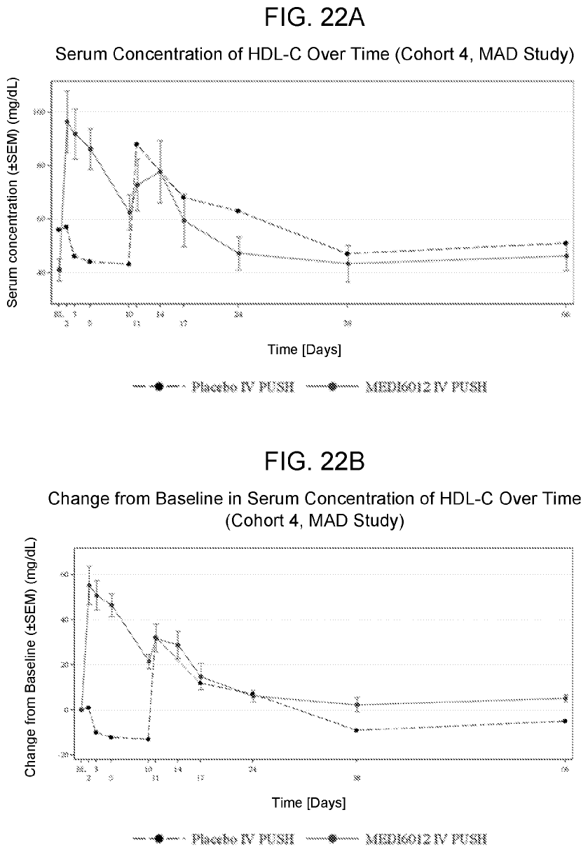

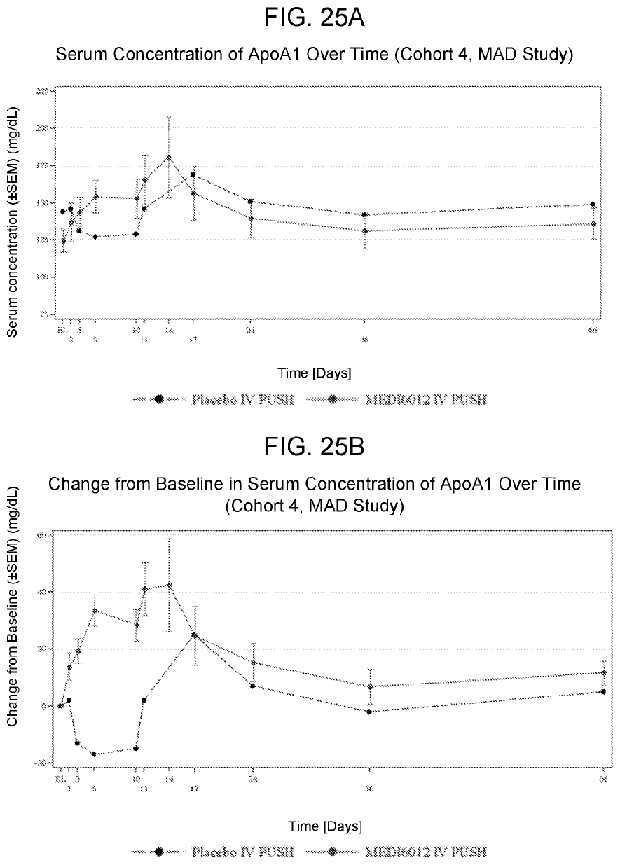

Analysis of the results from cohort 4 of the MAD study showed that the administration of MEDI6012 to study subjects in this dosing regimen, namely a dose of 300 mg of MEDI6012 administered to the subjects on Day 1 (loading dose); a second dose of 150 mg of MEDI6012 administered to the subjects at 48 hours (maintenance dose on Day 3); and a 100 mg dose of MEDI6012 administered to the subjects 1 week following the second dose (maintenance dose on Day 10), all administered by IV push, led to an increase in HDL-C and ApoA1 that is consistent with the mechanism of action of the LCAT enzyme (FIGS. 22A and 22B and FIGS. 25A and 25B, and Tables 6c-e). It is important to note that each subject is their own control when baseline corrected.

TABLE 6c
Baseline-adjusted AUC (0-96 h) in
HDL-C; As-treated Population (IV Push)

| | | Placebo IV Push | MEDI6012 IV Push |
|---|---|---|---|
| Dose # | | (N = 1) | (N = 7) |
| Post dose 1 | N | 1 | 7 |
| | Mean (SD) | −675.69 (N/A) | 4125.69 (552.08) |
| | Median | −675.69 | 4402.63 |
| Post dose 3 | N | 1 | 7 |
| | Mean (SD) | 266.68 (N/A) | 1803.12 (1684.32) |
| | Median | 266.68 | 658.73 |

TABLE 6d
Baseline-adjusted AUC (0-168 h) in
HDL-C; As-treated Population (IV Push)

| | | Placebo IV Push | MEDI6012 IV Push |
|---|---|---|---|
| Dose # | | (N = 1) | (N = 7) |
| Post dose 1 | N | 1 | 7 |
| | Mean (SD) | −1561.29 (N/A) | 6507.86 (1403.94) |
| | Median | −1561.29 | 6296.23 |
| Post dose 3 | N | 1 | 7 |
| | Mean (SD) | 3444.21 (N/A) | 3065.88 (2487.56) |
| | Median | 3444.21 | 2938.86 |

TABLE 6e
Baseline-adjusted AUC (0-96 h) in
ApoA1; As-treated Population (IV Push)

| | | Placebo IV Push | MEDI6012 IV Push |
|---|---|---|---|
| Dose # | | (N = 1) | (N = 7) |
| Post dose 1 | N | 1 | 7 |
| | Mean (SD) | −1208.16 (N/A) | 1669.34 (691.14) |
| | Median | −1208.16 | 2007.14 |
| Post dose 3 | N | 1 | 7 |
| | Mean (SD) | −20.42 (N/A) | 2399.42 (2611.21) |
| | Median | −20.42 | 744.70 |

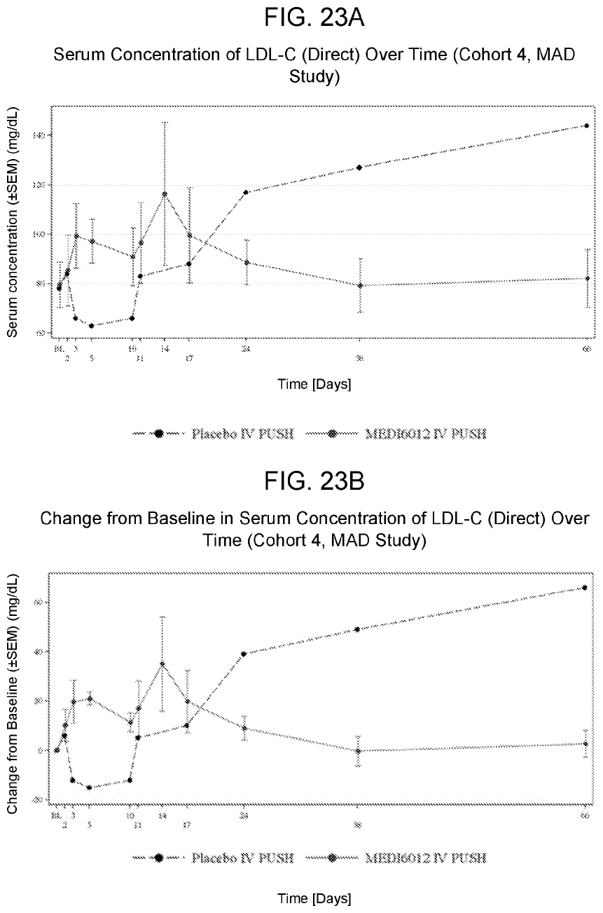

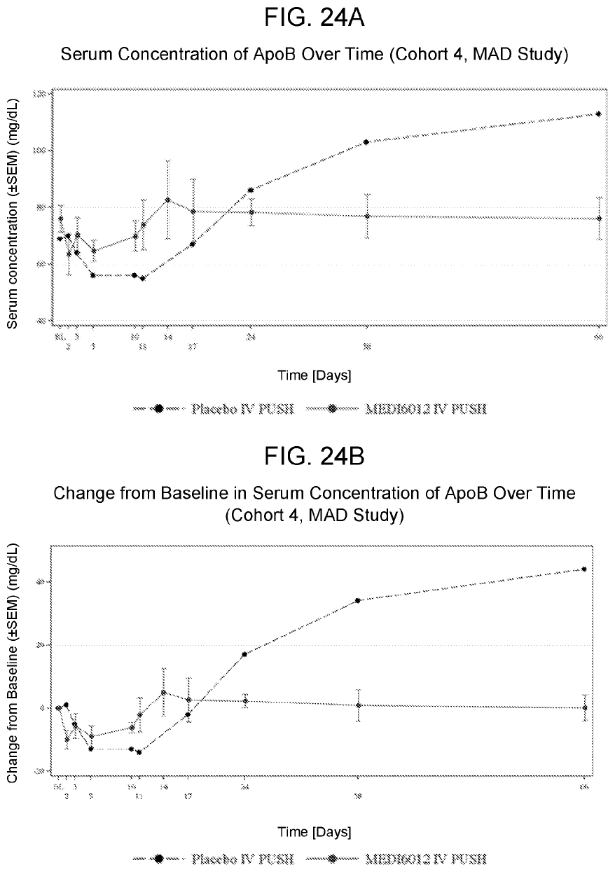

An increase in LDL levels was observed (FIGS. 23A and 23B and Table 61) as well as an initial decrease in apoB that returned to baseline following the third dose (FIGS. 24A and 24B and Table 6g). Overall, there was an increase in LDL cholesterol content, but no increase in LDL particle number.

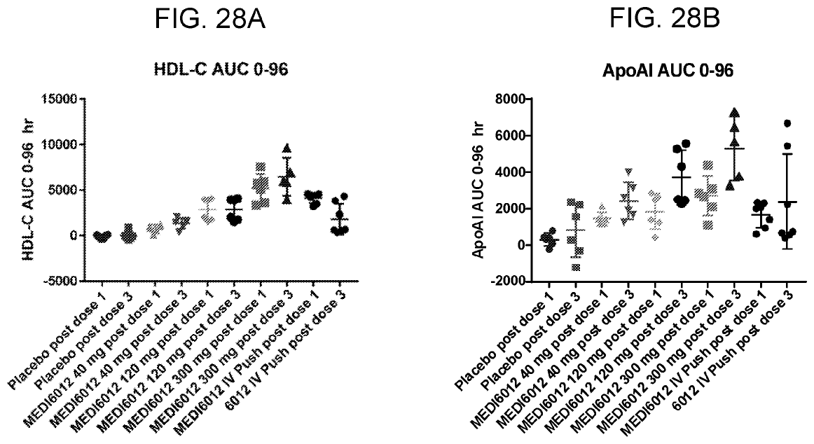

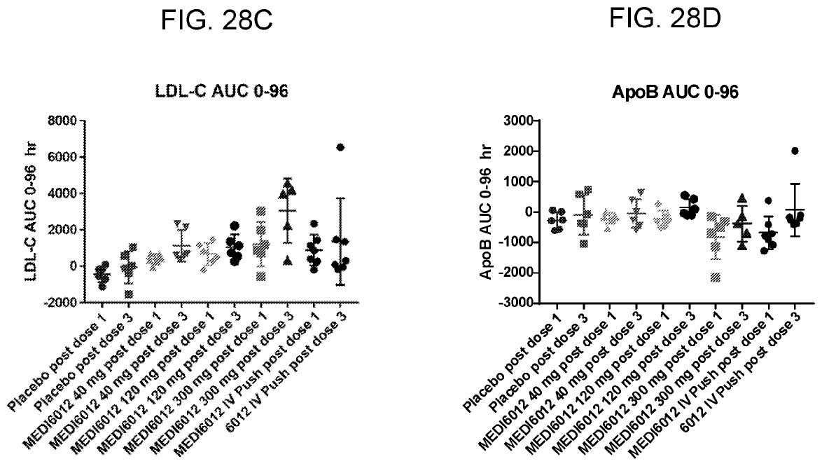

FIGS. 28A-28D present area under the concentration curve (AUC) box plots showing HDL-C, ApoA1, LDL-C and ApoB levels in subjects from Cohorts 1-4 following administration of MEDI6012 as described for the MAD study in Examples 2 and 3 herein. Dose-dependent increases in HDL-C and ApoA1 were observed (see FIGS. 28A and 28B). An increase in LDL-C was observed after the first 120 mg dose of MEDI6012 and after the third dose of both 40 mg and 120 mg (see FIG. 28C). An increase in LDL-C was also observed for both doses of 300 mg of MEDI6012 (Cohort 3) and for Cohort 4 (IV Push) of the MAD study. However, the LDL-C increases were not considered detrimental in view of the static (or decreased) levels of ApoB that were concomitantly measured in the subjects (see FIG. 28D). As no increases in ApoB were observed, this indicated that there was no detrimental increase in LDL particles associated with the MEDI6012 doses and dosing regimens.

TABLE 6f
Baseline-adjusted AUC (0-96 h) in LDL-C (Direct);
As-treated Population (IV Push)

| | | Placebo IV Push | MEDI6012 IV Push |
|---|---|---|---|
| Dose # | | (N = 1) | (N = 7) |
| Post dose 1 | N | 1 | 7 |
| | Mean (SD) | −616.78 (N/A) | 890.25 (840.94) |
| | Median | −616.78 | 871.57 |
| Post dose 3 | N | 1 | 7 |
| | Mean (SD) | −78.79 (N/A) | 1364.12 (2375.93) |
| | Median | −78.79 | 302.30 |

TABLE 6g
Baseline-adjusted AUC (0-96 h) in ApoB; As-treated Population (IV Push)

| | | Placebo IV Push | MEDI6012 IV Push |
|---|---|---|---|
| Dose # | | (N = 1) | (N = 7) |
| Post dose 1 | N | 1 | 7 |
| | Mean (SD) | −651.36 (N/A) | −681.99 (543.82) |
| | Median | −651.36 | −779.18 |
| Post dose 3 | N | 1 | 7 |
| | Mean (SD) | −239.17 (N/A) | 73.0 (864.30) |
| | Median | −239.17 | −209.54 |

As discussed previously, a number of reports have shown that apoB is a better predictor of risk of CHD than LDL-C in both men and women and that the number of atherogenic particles, such as apoB, is a more important indicator of risk than the amount of cholesterol (LDL-C) transported in these particles. (reviewed in Vaverkova, H., 2011, Clin Lipidology, 6(1):35-48; Sniderman, A D et al., 2003, Lancet, 361:777-780). Because all potentially atherogenic lipoprotein particles contain only one molecule of apoB and various amounts of cholesterol, apoB serves as a better marker of atherogenic lipoprotein particle numbers than LDL-C. Therefore, the little to no increase in apoB in subjects administered LCAT (MEDI6012) as demonstrated herein reflects a highly beneficial and protective treatment despite an observed increase in LDL-C.

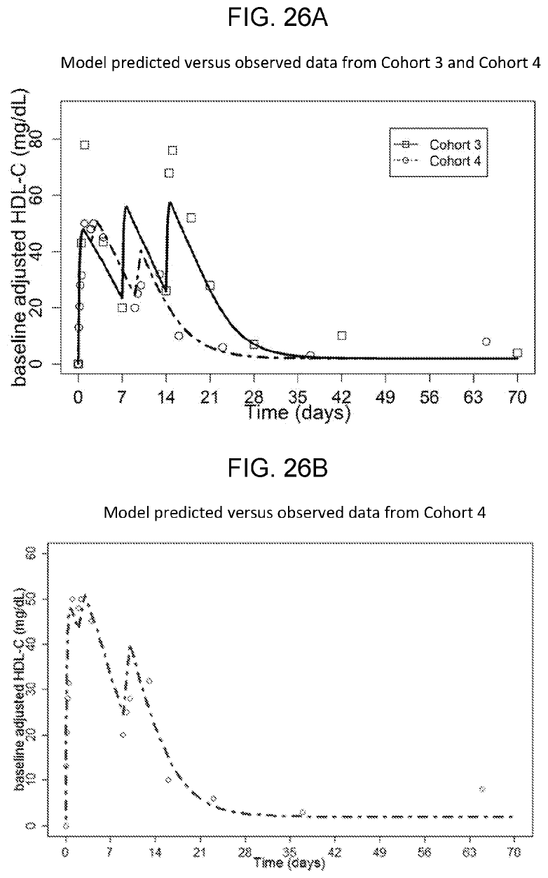

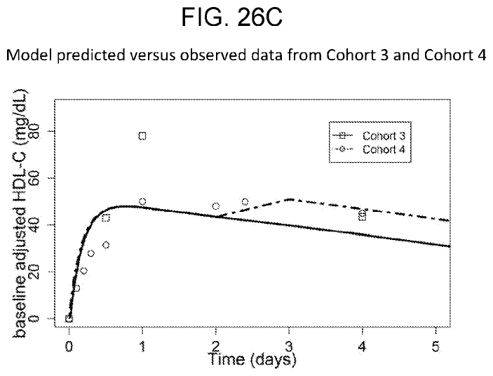

FIG. 26A shows a comparison of the baseline adjusted levels of HDL-C obtained from modelling/simulation analyses (the solid and dashed lines) compared to the observed data (the individual data points: circles and squares) from administration of MEDI6012 following the dosage regimen of Cohort 3 and Cohort 4 of the MAD study (Day 0 to Day 70). When the observed data from Cohort 4 is shown alone, the three distinct peaks of HDL-C can be observed following administration of MEDI6012 (FIG. 26B). FIG. 26C shows the data for Day 0 to Day 5 from FIG. 26A. From a comparison of these data, it can be seen that the initial modelling performed on the data from the SAD study and from Cohorts 1-2 of the MAD study is highly predictive of actual observed data.

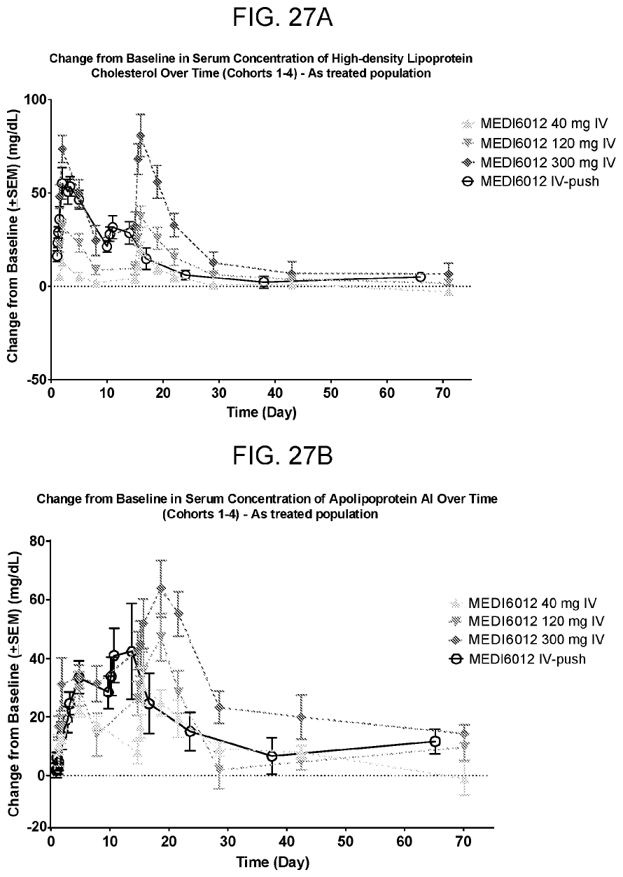

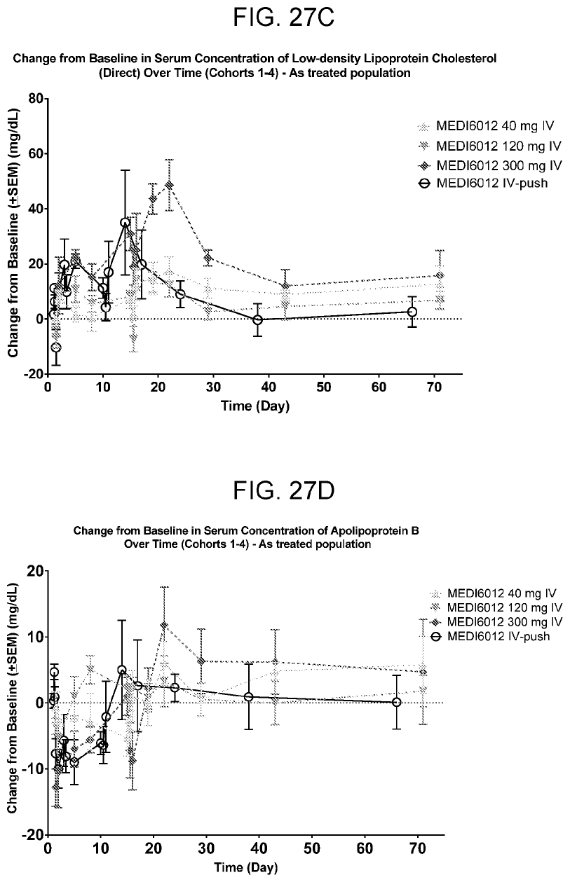

FIGS. 27A-D show the observed results from all cohorts (Cohorts 1-4) of the MAD study, as defined in Examples 2 and 3 herein. FIG. 27A shows the observed change from baseline in serum concentration of HDL-C over time from Cohorts 1-4 of the MAD study. FIG. 27B shows the observed change from baseline in serum concentration of ApoA1 over time from Cohorts 1-4 of the MAD study. FIG. 27C shows the observed change from baseline in serum concentration of LDL-C (Direct) over time from Cohorts 1-4 of the MAD study. FIG. 27D shows the observed change from baseline in serum concentration of ApoB over time from Cohorts 1-4 of the MAD study.

### Example 4

### A Phase 2b Randomized, Single Blind, Placebo-Controlled Trial to Evaluate the Safety and Efficacy of MEDI6012 in Acute ST Elevation Myocardial Infarction (STEMI)

### Study Design

A Phase 2b randomized, single blind, placebo controlled trial was designed to evaluate the safety and efficacy of MEDI6012 for the reduction in myocardial infarct (MI) size in subjects with acute STEMI compared with placebo and in addition to standard of care. This study randomizes up to 414 subjects at approximately 40 sites. It is expected that up to 60% of subjects have Thrombosis in Myocardial Infarction (TIMI) 0-1 flow and have completed MR imaging. Therefore, the goal is to have 252 subjects completing the study and included in the analyses for the primary outcome.

Subjects are randomized in a 1:1 ratio to one of 2 regimens (2-dose regimen or 6-dose regimen). Within each dose regimen, subjects are randomized in a 2:1 ratio to receive MEDI6012 or placebo. In the event that a dose regimen is dropped at the interim analysis, subjects are then randomized in a 1:1 ratio to receive MEDI6012 or placebo for the remaining dose regimen. While the trial enrolls acute ST elevation myocardial infarction (STEMI) patients from all three vascular territories, non-anterior STEMI is limited to <50% of the enrolled subjects. Anterior STEMI is defined when the culprit vessel is the left main or left anterior descending arteries or their branches (anomalous origins included). Subjects are screened for eligibility upon arrival to the hospital for acute STEMI care. Following informed consent/verbal assent (according to local ethics board requirements) for the first infusion of investigational product, subjects receive a loading dose of investigational product via intravenous (IV) push prior to primary percutaneous coronary intervention (PCI; Day 1), preferably 10 minutes prior to balloon inflation in the culprit vessel. In addition, it is recommended that all subjects begin high intensity statin therapy as early as possible and according to local standards. On Day 1-3, subjects provide written informed consent for the remaining study procedures, which include a CMR, infusions of investigational product, blood sampling and testing and, in some cases, coronary CTA.

The study tests two dosing regimens. In the 2-dose regimen arms, the first dose of MEDI6012 is given prior to primary PCI and a second dose is given 48 hours (±8 hours) later, all during the inpatient visit. In the 6 dose regimen arms, the first dose of MEDI6012 is given prior to primary PCI and a second dose is given 48 hours±8 hours later, both during the inpatient visit. These doses are followed by 4 additional weekly doses (±1 day) given as an outpatient

Evaluated outcomes of the study include the hypotheses that administration of MEDI6012 in the study doses reduces myocardial infarct compared with placebo; improves systolic function (ejection fraction (EF) of the left ventricle); induces regression and reduces progression of non-calcified coronary plaque compared with placebo; exhibits an acceptable safety and immunogenicity profile in subjects with acute STEMI; reduces ischemia/reperfusion injury; and prevents adverse remodeling of the left ventricle (LV).

The target study population includes adult men or women, aged 30-80, who present to the hospital with a diagnosis of acute STEMI on a 12-lead electrocardiogram (ECG) with planned primary PCI within 6 hours of most recent symptom onset (i.e., continuous symptoms for less than 6 hours). Women of child-bearing potential are excluded.

### Treatment Groups and Regimens

1) 2-Dose Regimen Randomized 2:1 to the following treatments: MEDI6012 300 mg IV push on Day 1, and 150 mg IV push on Day 3 (48 hours (±8 hours)) Placebo IV push on Day 1 and Day 3 (48 hours (±8 hours));

2) 6-Dose Regimen Randomized 2:1 to the following treatments: MEDI6012 300 mg IV push on Day 1, 150 mg IV push on Day 3 (48 hours (±8 hours)), and 100 mg on days 10, 17, 24, and 31;

3) Placebo IV push on Day 1, Day 3 (48 hours (±8 hours)), and Days 10, 17, 24, and 31.

In the study, the MEDI6012 treatment groups include 138 enrolled subjects to ensure at least 82 subjects complete treatment and primary endpoint study procedures meeting the definition of the “per-protocol, primary analysis population.” Each placebo group has 69 subjects per dosing regimen resulting in 138 subjects treated with placebo (82 subjects completing treatment and primary endpoint study procedures). The “intention-to-treat” (ITT) population” includes all randomized subjects. The “as-treated population” includes all randomized subjects receiving at least 1 dose of investigational product. The “primary efficacy analysis population” includes all randomized subjects receiving a full treatment course with investigational product with TIMI flow grade 0 or 1. The “efficacy analysis population—TIMI 2-3” includes all randomized subjects receiving a full treatment course of investigational product with TIMI flow grade 2 or 3. The “efficacy analysis population—TIMI 0-3” includes all randomized subjects receiving a full treatment course of investigational product with TIMI flow grade 0 to 3. The “CTA analysis population” includes randomized subjects in a 6-dose regimen receiving a full treatment course of investigational product.

### Statistical Methods

Sample Size

A total of 82 subjects per arm provide 80% power to detect a 25% reduction in infarct size between MEDI6012 2-dose group and placebo group and between MEDI6012 6-dose group and placebo group, with two-sided alpha 0.05 assuming a coefficient of variation (CV) of 0.65. A 40% rate of exclusion from the primary efficacy analysis population is expected due to TIMI grade 2 or 3 flow in the infarct-related artery on initial angiography and other reasons for subsequent exclusion or drop-out (Botker H E et al, 2010, Lancet, 375:727-734. Hausenloy D T, et al., 2013, Cardiovascular Research, 98, 7-27), a total of 138 subjects per arm is required. With this sample size, the power to detect a 5% absolute difference in EF between MEDI6012 group and placebo group is 88% assuming standard deviation 10%. For the secondary endpoint of non-calcified coronary plaque regression/progression, there will be >80% power to detect a 12 mm³ change in non-calcified plaque volume from index CTA to the 10-12 week CTA between subjects in the group doses with MEDI6012 and those in the placebo group, assuming a common standard deviation of 25% and 20% drop-out.

Statistical Analyses:

The primary efficacy endpoint of infarct size is analyzed using t-test with log-transformation of the data based on the primary efficacy analysis population. The endpoint of infarct size is also analyzed based on the efficacy analysis population—TIMI 2-3, the efficacy analysis population—TIMI 0-3, and ITT population. Ejection fraction, myocardial mass and left ventricular volumes are analyzed similarly to infarct size without the log-transformation of the data. Change from index CTA in non-calcified plaque volume is analyzed using t-test based on CTA analysis population. Area under the creatine kinase curves from 0-48 hours with log-transformation is analyzed using t-test based on as-treated population.

Safety analyses are based on the As-treated Population. Adverse event (AE) and serious adverse event (SAE) collection begins after the subject signs the informed consent document and lasts until the end of study visit. Treatment-emergent AEs (TEAEs) and treatment-emergent SAEs (TESAEs) are coded by the most updated version of the Medical Dictionary for Regulatory Activities (MedDRA), and the type incidence, severity, and relationship to investigational product are summarized. Specific AEs are counted once for each subject for calculating percentages. In addition, if the same AE occurs multiple times within a particular subject, the highest severity and level of relationship observed is reported. All TEAEs and TESAEs are summarized overall, as well as categorized by MedDRA system organ class and preferred term.

Vital sign results are summarized using descriptive statistics at each time point by treatment group. Electrocardiogram (ECG) parameters are also assessed and summarized descriptively by treatment group. Anti-drug antibody (ADA) incidence rate and titer are tabulated for each treatment group. Samples confirmed positive for ADA are tested and analyzed for nAb and summarized similarly.

Interim Analysis:

Two interim analyses are planned The objective of the first interim analysis is for futility and potentially dropping a dose regimen. It is conducted after 30% of the primary analysis population has completed their final study visit. The second interim analysis is planned to accelerate decision on future development options for MEDI6012 and is performed once 60% of the subjects have completed their final study visit.

### Methods for Assigning Treatment Groups

An interactive voice/web response system (IXRS) is used for randomization of subjects to a treatment regimen and group and assignment of blinded investigational product kit numbers. A subject is considered randomized into the study when the investigator notifies the IXRS that the subject meets eligibility criteria and the IXRS provides the assignment of blinded investigational product kit numbers to the subject.

Subjects are randomized in a 1:1 ratio to one of 2 regimens (2-dose Regimen or 6-dose Regimen). Within each dose regimen, subjects are randomized in a 2:1 ratio to receive MEDI6012 or placebo:
MEDI6012, 6-dose Regimen (N=138)
Placebo, 6-dose Regimen (N=69)
MEDI6012, 2-dose Regimen (N=138)
Placebo, 2-dose Regimen (N=69)

In the event that a dose regimen is dropped at the interim analysis, subjects are randomized in a 1:1 ratio to receive MEDI6012 or placebo for the remaining dose regimen. If there is a delay in the administration of investigational product such that it is not administered within the specified timeframe, the medical monitor must be notified immediately.

The distribution of patients with anterior vs. non-anterior MIs is monitored over the course of the study. The goal is that ˜50% of the final randomized population is anterior MI. Therefore, the number with non-anterior MI is monitored via the IXRS.

### Rationale for Endpoints

Primary Endpoint:

Infarct size as a percentage of LV mass measured on delayed-enhanced cardiovascular magnetic resonance (CMR) imaging 10-12 weeks post-MI.

Rationale:

CMR is considered the gold standard for the evaluation of infarct size and is considered the most relevant endpoint in cardioprotection trials (Hausenloy D T, et al., 2013, Cardiovascular Research, 98:7-27). Infarct size measured at 10-12 weeks reflects final infarct size after remodeling of the left ventricle (LV) and will reflect both the early and late effects of treatment with MEDI6012 (Mather A N, et al., 2011, Radiology, 261(1):116-26). Infarct size is measured on gadolinium delayed-enhanced MR images as the infarct size in grams divided by LV mass in grams. Infarct size is an independent predictor of secondary major adverse cardiovascular events, including mortality and hospitalization for heart failure (Stone G W, et al., 2016, J Am Coll Cardiol., 67(14):1674-83; Wu E, et al., 2008, Heart, 94:730-736.) For every 5% increase in infarct size, there is a 19% increase risk of all-cause mortality and a 20% increase risk of heart failure hospitalization (Stone G W, et al., 2016, JAm Coll Cardiol., 67(14):1674-83).

### Secondary Endpoints:

Ejection fraction (EF) measured by cine MR imaging at 10-12 weeks post-MI compared to placebo.

Rationale:

Ejection fraction is a well-established measurement of the systolic function of the LV. Additionally, pharmacologic improvements in EF have been linked to decreases in mortality and heart failure hospitalizations (Kramer D, et al., 2010, J Am Coll Cardiol., 56:392-406; Breathett K, et al., 2016, Circ Heart Fail., 9:e002962). EF is calculated as the ratio of stroke volume divided by end-diastolic volume.

Change, from index CTA, in non-calcified plaque volume (NCPV) is measured by end of study CTA in the coronary arteries 10-12 weeks post-MI compared to placebo.

Rationale:

When studied in acute coronary syndrome (ACS), specifically non-STEMI ACS, non-calcified plaque volume (NCPV) is a better predictor of major adverse cardiac events

(MACE) when compared to Agatston calcium score and total plaque volume. NCPV is measured in all vessels ≥2 mm in diameter and expressed in mm³. Coronary segments with stents or otherwise deemed uninterpretable will be excluded from analysis.

Area Under the Creatinee Kinase (CK) Curves from 0-48 Hours

Rationale:

This result aids in determining if the effect of MEDI6012 is mainly the first dose versus multiple doses given over a 6 week period.

### Myocardial Mass and LV Volumes at End-Systole and End-Diastole

Rationale:

LV volumes and myocardial mass are well-established predictors of clinical outcomes and will be are measured by cardiovascular magnetic resonance (CMR) imaging. Ventricular volumes and myocardial mass are measured in mL and g, respectively, and indexed to body surface area.

Safety and tolerability of MEDI6012 is measured by the incidence of treatment-emergent adverse events (TEAEs) and treatment-emergent serious adverse events (TESAEs), and anti-drug antibodies (ADAs), and neutralizing antibodies over time to last study visit, Days 70-84.

Rationale:

Further safety and tolerability data support further drug development.

### PK and Immunogenicity as Measured by LCAT Mass and ADAs

Rationale:

Further PK and immunogenicity data support dose rationale in future studies and further drug development.

### Exploratory Endpoints

Non-Calcified Atherosclerotic Plaque Volume in the Aorta. Rationale:

MEDI6012 has the potential to cause regression of atheroma in vessels outside of the heart. During a coronary CTA; the aortic root, proximal ascending aorta, and most of the descending thoracic aorta are imaged. Similar to the coronary arteries, non-calcified atherosclerotic plaque volume is measured in the aorta to determine if MEDI6012 can regress atheroma outside of the heart.

### Rationale for Dose(s) Selected

Results from the single ascending and multiple ascending dose studies of MEDI6012 as described in Examples 1 and 2 supra demonstrated that the rate of the increases in HDL-C and apoA1 are dose-dependent. Preclinical studies established that infusions of ApoA1 or HDL particles confer myocardial protection during acute STEMI. Since this study involves treating STEMI patients in the acute setting, the aim is to increase HDL and apoA1 as rapidly as possible. Therefore, on Day 1 a loading dose of 300 mg of MEDI6012 or placebo is administered to achieve a rapid rise in HDL-C.

Based on data and modeling from the MEDI6012 single ascending dose (SAD) study and cohorts 1 and 2 from the multiple ascending dose (MAD) study, a 300 mg dose is expected to increase HDL-C by ˜50% in 90 minutes and ˜100% in 6 hours (assuming a mean HDL-C of 35 mg/dL in STEMI patients). In addition, this dose is expected to improve HDL function based on cholesterol efflux capacity data from the single ascending dose study and to cause a minimal rise in very, very large HDL (VVL-HDL) particles (>17 nm). A second dose of 150 mg of MEDI6012 or placebo is administered 48 hours (approximately one half-life) following the first dose in order to maintain HDL-C and/or apoA1 levels/concentration during the acute and subacute phases of myocardial infarction.

For the 2-dose regimen (Table 7 below), dosing stops after the second dose, with dosing occurring in the inpatient setting only. This regimen has the advantage of being a short duration therapy that does not require the subject to return for further infusions as an outpatient. For the 6-dose regimen (Table 8 below), subjects receive the baseline 300 mg dose and the 150 mg dose at 48 hours, followed by 4 weekly 100-mg doses as an outpatient. A maintenance dose of 100 mg was selected to maintain HDL-C at a level conferring benefit in epidemiology studies and at a level that does not result in an appreciable accumulation of CE in LDL particles.

TABLE 7
2-Dose Regimen

| Dose | Administration |
|---|---|
| Day 1-Dose #1 (Loading Dose) | 300 mg of MEDI6012 infused via |
| | IV push over 1-2 minutes |
| Day 3 (48 ± 8 hours)-Dose #2 | 150 mg of MEDI6012 infused via |
| (Inpatient Maintenance Dose) | IV push over 1-2 minutes |

IV = intravenous.

TABLE 8
6-Dose Regimen

| Doses | Administration |
|---|---|
| Day 1-Dose #1 (Loading Dose) | 300 mg of MEDI6012 infused via |
| | IV push over 1-2 minutes |
| Day 3 (48 ± 8 hours)-Dose #2 | 150 mg of MEDI6012 infused via |
| (Inpatient Maintenance Dose) | IV push over 1-2 minutes |
| Day 10, 17, 24, 31-Doses 3-6 | 100 mg of MEDI6012 infused via |
| (Outpatient Maintenance Doses) ^{a} | IV push over 1-2 minutes |

IV = intravenous; STEMI = ST elevation myocardial infarction.
Doses 3-6 have a window of ±1 Day to account for STEMIs occurring on Saturday or Sundays.

### Statistical Analyses

### Efficacy Analyses:

The primary efficacy endpoint of infarct size is analyzed using t-test with log-transformation of the data based on the primary efficacy analysis population. The primary efficacy endpoint of infarct size is also analyzed based on the efficacy analysis population—TIMI 2-3, the efficacy analysis population—TIMI 0-3, and ITT population. Ejection fraction (EF), myocardial mass, and left ventricular (LV) volumes are analyzed in a manner similar to that of infarct size without the log-transformation of the data. Change from index computed tomography angiography (CTA) in non-calcified plaque volume is analyzed using t-test based on CTA analysis population. Area under the creatine kinase curves from 0-48 hours with log-transformation is analyzed using t-test based on as-treated population.

### Interim Analysis:

Two interim analyses are planned The objective of the first interim analysis is for futility and potentially dropping a dose regimen. It is conducted after 30% of the primary analysis population has completed its final study visit. The second interim analysis is planned to accelerate decision on future MEDI6012 development and is performed once 60% of the subjects have completed their final study visit. Details of the interim analyses are specified in the interim analysis plan prior to unblinding.

### OTHER EMBODIMENTS

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1. A method of treating heart disease or cardiovascular disease and/or the symptoms thereof in a subject, the method comprising:
administering to a subject in need thereof one or more doses of an isolated and purified lecithin-cholesterol acyltransferase (LCAT) enzyme in a dosage amount of 20-2000 mg, wherein each dose is administered over a time period of about 1 minute to about 3 hours to treat heart disease or cardiovascular disease and/or the symptoms thereof in the subject.

2. The method of claim 1, wherein the subject has acute or chronic heart disease, cardiovascular disease, coronary artery disease (CAD), stable CAD, atherosclerosis, atherosclerotic cardiovascular disease (CVD), stable CVD, unstable CVD, acute coronary syndrome (ACS), stroke, ischemic stroke, myocardial disease, myocardial infarction, familial or acquired, heart failure with reduced ejection fraction (EF), heart failure with preserved EF, non-ischemic cardiomyopathy, chemotherapy-induced cardiomyopathy, cerebrovascular disease, acute or chronic renal disease and/or symptoms thereof.

3. (canceled)

4. (canceled)

5. The method of claim 1, wherein the one or more doses of LCAT are administered to the subject in an amount selected from 300 mg, 150 mg and 100 mg.

6. The method of claim 5, wherein the one or more doses of LCAT comprise a first dose in an amount of 300 mg and a second dose in an amount of 150 mg administered about 48 hours±8 hours following the first dose.

7. The method of claim 5, wherein the one or more doses of LCAT comprise a first dose in an amount of 300 mg; a second dose in an amount of 150 mg administered about 48 hours±8 hours following the first dose; and a third dose in an amount of 100 mg administered about a week following the second dose.

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. The method of claim 1, wherein the administration of LCAT via subcutaneous (SC) injection or IV push increases endogenous levels of high density lipoprotein-cholesterol (HDL-C) and/or apolipoprotein A1 (apoA1) in the subject.

25. The method of claim 24, wherein the administration of LCAT by SC injection at a dose of 600 mg increases endogenous levels of apolipoprotein A1 (apoA1) in the subject.

26. The method of claim 24, wherein the administration of LCAT does not increase endogenous levels of apolipoprotein B (apoB) in the subject.

27. The method of claim 1, wherein the isolated and purified LCAT is recombinant human LCAT (rhLCAT).

28. The method of claim 27, wherein the rhLCAT is MEDI6012 (SEQ ID NO: 2).

29. A method of treating heart disease or cardiovascular disease and/or the symptoms thereof in a subject, the method comprising:
administering to a subject in need thereof a loading dose of an isolated and purified lecithin-cholesterol acyltransferase (LCAT) enzyme in an amount of 250-500 mg delivered to the subject by intravenous (IV) push over a time period of about 1-5 minutes upon presentation of the subject for treatment.

30. The method of claim 29, wherein the loading dose of LCAT is administered to the subject in an amount of 300 mg.

31. (canceled)

32. (canceled)

33. (canceled)

34. The method of claim 30, wherein a dose of LCAT in an amount of 100-200 mg is administered to the subject following the loading dose.

35. (canceled)

36. (canceled)

37. (canceled)

38. The method of claim 34, wherein at least 4 weekly doses of LCAT in an amount of 80-150 mg per dose are administered to the subject about a week following the 100-200 mg dose or the 150 mg dose.

39. The method of claim 34, wherein at least 4 weekly doses of LCAT in an amount of 100 mg per dose are administered to the subject about a week following the 100-200 mg dose or the 150 mg dose.

40. The method of claim 29, wherein the one or more doses of LCAT following the loading dose are intravenously administered to the subject by IV push and/or IV infusion.

41. The method of claim 29, wherein the isolated and purified LCAT is recombinant human LCAT (rhLCAT).

42. The method of claim 41, wherein the rhLCAT is MEDI6012 (SEQ ID NO: 2).

43. (canceled)

44. (canceled)

45. The method of claim 40, wherein three doses of LCAT are administered to the subject and comprise a dose of 300 mg administered on day 1; a dose of 150 mg administered on day 3; a dose of 100 mg administered on day 10; and optionally wherein subsequent doses of LCAT are administered to the subject at predetermined time intervals up to about 30 days, or longer, following the day 10 dose.

46. (canceled)

47. The method claim 29, wherein the subject has acute or chronic heart disease, cardiovascular disease, coronary artery disease (CAD), stable CAD, atherosclerosis, atherosclerotic cardiovascular disease (CVD), stable CVD, unstable CVD, acute coronary syndrome (ACS), stroke, ischemic stroke, myocardial disease, myocardial infarction, familial or acquired, heart failure with reduced ejection fraction (EF), heart failure with preserved EF, non-ischemic cardiomyopathy, chemotherapy-induced cardiomyopathy, cerebrovascular disease, acute or chronic renal disease and/or symptoms thereof.

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. (canceled)

78. (canceled)

79. (canceled)

80. (canceled)

81. (canceled)

82. (canceled)

83. (canceled)

84. (canceled)

85. (canceled)

86. (canceled)

87. (canceled)

88. (canceled)

89. (canceled)

90. (canceled)

91. (canceled)

92. (canceled)

93. (canceled)

94. (canceled)

95. (canceled)

96. (canceled)

97. (canceled)

98. (canceled)

99. (canceled)

100. (canceled)

101. (canceled)

102. (canceled)

103. (canceled)

104. (canceled)

105. (canceled)

106. (canceled)

107. (canceled)

108. (canceled)

109. (canceled)


SEQUENCE LISTING (3 sequences)

SEQ ID NO: 1 - protein, 440 aa, Homo sapiens
MGPPGSPWQWVTLLLGLLLPPAAPFWLLNVLFPPHTTPKAELSNHTRPVILVPGCLGNQLEAKLDKPDVVNWMCYRKTEDFFTIWLDLNMFLPLGVDCWIDNTRVVYNRSSGLVSNAPGVQIRVPGFGKTYSVEYLDSSKLAGYLHTLVQNLVNNGYVRDETVRAAPYDWRLEPGQQEEYYRKLAGLVEEMHAAYGKPVFLIGHSLGCLHLLYFLLRQPQAWKDRFIDGFISLGAPWGGSIKPMLVLASGDNQGIPIMSSIKLKEEQRITTTSPWMFPSRMAWPEDHVFISTPSFNYTGRDFQRFFADLHFEEGWYMWLQSRDLLAGLPAPGVEVYCLYGVGLPTPRTYIYDHGFPYTDPVGVLYEDGDDTVATRSTELCGLWQGRQPQPVHLLPLHGIQHLNMVFSNLTLEHINAILLGAYRQGPPASPTASPEPPPPE

SEQ ID NO: 2 - protein, 416 aa, Homo sapiens
FWLLNVLFPPHTTPKAELSNHTRPVILVPGCLGNQLEAKLDKPDVVNWMCYRKTEDFFTIWLDLNMFLPLGVDCWIDNTRVVYNRSSGLVSNAPGVQIRVPGFGKTYSVEYLDSSKLAGYLHTLVQNLVNNGYVRDETVRAAPYDWRLEPGQQEEYYRKLAGLVEEMHAAYGKPVFLIGHSLGCLHLLYFLLRQPQAWKDRFIDGFISLGAPWGGSIKPMLVLASGDNQGIPIMSSIKLKEEQRITTTSPWMFPSRMAWPEDHVFISTPSFNYTGRDFQRFFADLHFEEGWYMWLQSRDLLAGLPAPGVEVYCLYGVGLPTPRTYIYDHGFPYTDPVGVLYEDGDDTVATRSTELCGLWQGRQPQPVHLLPLHGIQHLNMVFSNLTLEHINAILLGAYRQGPPASPTASPEPPPPE

SEQ ID NO: 3 - DNA, 1323 bp, Homo sapiens
atggggccgcccggctccccatggcagtgggtgacgctgctgctggggctgctgctccctcctgccgcccccttctggctcctcaatgtgctcttccccccgcacaccacgcccaaggctgagctcagtaaccacacacggcccgtcatcctcgtgcccggctgcctggggaatcagctagaagccaagctggacaaaccagatgtggtgaactggatgtgctaccgcaagacagaggacttcttcaccatctggctggatctcaacatgttcctaccccttggggtagactgctggatcgataacaccagggttgtctacaaccggagctctgggctcgtgtccaacgcccctggtgtccagatccgcgtccctggctttggcaagacctactctgtggagtacctggacagcagcaagctggcagggtacctgcacacactggtgcagaacctggtcaacaatggctacgtgcgggacgagactgtgcgcgccgccccctatgactggcggctggagcccggccagcaggaggagtactaccgcaagctcgcagggctggtggaggagatgcacgctgcctatgggaagcctgtcttcctcattggccacagcctcggctgtctacacttgctctatttcctgctgcgccagccccaggcctggaaggaccgctttattgatggcttcatctctcttggggctccctggggtggctccatcaagcccatgctggtcttggcctcaggtgacaaccagggcatccccatcatgtccagcatcaagctgaaagaggagcagcgcataaccaccacctccccctggatgtttccctctcgcatggcgtggcctgaggaccacgtgttcatttccacacccagcttcaactacacaggccgtgacttccaacgcttctttgcagacctgcactttgaggaaggctggtacatgtggctgcagtcacgtgacctcctggcaggactcccagcacctggtgtggaagtatactgtctttacggcgtgggcctgcccacgccccgcacctacatctacgaccacggcttcccctacacggaccctgtgggtgtgctctatgaggatggtgatgacacggtggcgacccgcagcaccgagctctgtggcctgtggcagggccgccagccacagcctgtgcacctgctgcccctgcacgggatacagcatctcaacatggtcttcagcaacctgaccctggagcacatcaatgccatcctgctgggtgcctaccgccagggtccccctgcatccccgactgccagcccagagcccccgcctcctgaataa